(19) United States
(12) Patent Application Publication
(10) Pub. No.: US 2020/0206342 A1
(43) Pub. Date: Jul. 2, 2020

(54) ENGINEERED VARIANTS OF HIV-1 ENV FOR PRESENTATION OF QUARTENARY EPITOPES

(71) Applicant: THE BOARD OF TRUSTEES OF THE UNIVERSITY OF ILLINOIS, Urbana, IL (US)

(72) Inventors: Erik PROCKO, Champaign, IL (US); Jeremiah Dallas HEREDIA, Urbana, IL (US)

(73) Assignee: THE BOARD OF TRUSTEES OF THE UNIVERSITY OF ILLINOIS, Urbana, IL (US)

(21) Appl. No.: 16/631,275

(22) PCT Filed: Jul. 18, 2018
(86) PCT No.: PCT/US2018/042610

§ 371 Date: Jan. 15, 2020

Related U.S. Application Data
(60) Provisional application No. 62/534,191, filed on Jul. 18, 2017.

(51) Int. Cl. A61K 39/21; C12N 15/86

(52) U.S. Cl. CPC A61K 39/21; C12N 2740/16134; C12N 2740/16122; C12N 15/86

(57) ABSTRACT

Provided herein are HIV-1 Env proteins or fragments thereof comprising one or more amino acid mutations; and nucleic acid molecule encoding the same. Further provided is a method of screening a compound for binding to one or more mutant HIV-1 Env proteins; and methods for eliciting an immune response against an HIV-1 infected cell, comprising administering to a subject an amount of a mutant HIV-1 Env protein, a fragment thereof a mutant HIV-1 Env trimeric complex, or portion thereof, effective to elicit an immune response in the subject. Further provided is a pharmaceutical composition, such as a vaccine, comprising the mutant HIV-1 Env protein or fragment thereof.

### PRIORITY

This application claims the benefit of U.S. Ser. No. 62/534,191, filed Jul. 18, 2017, which is incorporated herein by reference in its entirety.

### STATEMENT REGARDING FEDERALLY SPONSORED RESEARCH OR DEVELOPMENT

This invention was made with government support under contract number R01AI129719 awarded by the National Institutes of Health. The government has certain rights in the invention.

### BACKGROUND

HIV-1 engages target cells through interactions between the viral spike glycoprotein Env and host receptors. HIV-1 Env is formed by a homotrimeric complex of gp160 subunits that are cleaved by host proteases during maturation into extracellular gp120 and membrane-tethered gp41, which remain non-covalently associated in a ‘closed’ conformation¹. During infection, the gp120 subunit binds the primary host receptor CD4, inducing a change to an ‘open’ conformation of Env that exposes binding sites for a secondary co-receptor²⁻⁴. This co-receptor is one of either two chemokine receptors, CCR5 or CXCR4, and once bound, further conformational changes release fusogenic regions of gp41 that mediate membrane fusion and viral entry into the host cell.

Env is the only viral protein on the outside of a HIV-1 virion accessible to the humoral immune system, and therefore has been extensively studied for vaccine development °. Mature Env has a complex structural organization. At the apex most distal from the membrane is a trimerization domain that mediates contacts between gp120 subunits, and is formed by variable regions V1, V2, and V3⁶⁻¹¹. In the central region of the Env spike are the gp120 inner and outer domains, which face in towards the trimer axis or out towards bulk solvent, respectively. Below, the N- and C-termini of gp120 are encircled and ‘grasped’ by the extracellular region of gp41, with the gp41 heptad repeat HR1 forming trimer contacts that resemble a helical-bundle ‘spine’ at the center of the complex. Next is a trimeric association of gp41 transmembrane helices¹², followed by gp41 cytoplasmic tails. Infection is initiated by CD4 binding to Env in a closed conformation, where there are close interactions between apical tips of the trimerization domains⁶. CD4 binding stabilizes large conformational changes that break apical contacts and promote opening of the structure^{6,13}. The closed and open conformations have distinct antigenic profiles, with many broadly neutralizing antibodies (bNAbs) binding preferentially to closed Env^{14,15}, while the open state presents strain-specific, poorly neutralizing epitopes in the V3 region^{16,17}.

Env sequence diversity, exposure of non-neutralizing or strain-specific immunodominant epitopes, and epitope shielding by extensive glycosylation, all act to limit potency and breadth of the host response. Design and purification of Env immunogens that correctly fold into native-like, pre-fusion closed trimers is also challenging, due to intrinsic conformational flexibility. The most notable engineered form of Env for vaccine purposes contains the so-called SOSIP mutations (an introduced disulfide between residues 501 and 605 to prevent gp120-gp41 dissociation¹⁸, and an I559P mutation that destabilizes the post-fusion trimeric conformation¹⁹; all residue numbers throughout are based on the HXB2 reference strain), which permit the expression and purification of soluble extracellular Env as native trimers^{20,21}. The best studied SOSIP construct is in the clade A strain BG505 sequence, but SOSIP mutations have also been introduced into Env from other strains in different clades. SOSIP constructs have been engineered with additional mutations and/or disulfide bonds to further stabilize the closed conformation recognized by most bNAbs²²⁻²⁴, reduce exposure of V3 region epitopes^{17,22-24}, be expressed as single chain non-proteolyzed native trimers^{25,27}, and to bind germline B cells with higher affinity to stimulate lineages that mature into potent bNAb secretors^{24,28}. More reductionist approaches have trimmed down the extracellular regions of Env to produce stable fragments of gp120^{29,30} or, at the most extreme, stabilize isolated epitopes for focusing an immune response³¹⁻³³. Understanding how Env sequence dictates conformation can therefore assist the design of sophisticated Env immunogens for optimum immunogenicity. Compared to these previous efforts, full or close-to-full length Env that contains the transmembrane domain has not been as extensively engineered to stabilize closed trimeric conformations. This is despite full-length Env being well suited for some vaccine formats, such as a DNA vaccine, virus-like particles (VLPs), or purified protein embedded in membrane nanodiscs.

Deep mutational scanning couples directed evolution of diverse sequence populations with next generation sequencing to track the phenotypic fitness of thousands of mutations simultaneously³⁴. The method has been used for vaccine design to screen for mutations within SOSIP constructs that enhance direct interactions with an antibody and its germline precursors, reduce exposure of V3 epitopes, and improve thermostability^{24,35 22}. Tissue culture propagation of viruses expressing Env variants has also been followed by next generation sequencing, and the mutational tolerance observed in these experiments closely aligns with observed diversity of natural Env sequences³⁶. However, this experimental system has proven insightful for characterizing Env mutations that allow HIV-1 to escape neutralization from a broadly-neutralizing antibody (bNAb)³⁷.

### DESCRIPTION OF THE DRAWINGS

FIG. 1. Sequence-activity landscapes of BaL Env interacting with protein ligands. Merged data from in vitro evolution of three SSM libraries that together fully span the mature Env_{BaL}t protein. The libraries were evolved by FACS for high binding signals to (A) 200 nM CD4(D1-D2), (B) 5 nM VRC01, and (C) 2 nM PG16. The Env sequence is on the vertical axis (HXB2 numbering, BaL numbering in parentheses), and single amino acid substitutions are on the horizontal axis. *, stop codon. Log₂ enrichment ratios are plotted from ≤−2 (depleted, black) to ≥+3 (enriched, white). Mutations missing in the libraries are black. The left schematic outlines sequence features of gp120 (dark grey) and gp41 (light grey), with an arrowhead indicating the proteolysis site. V1-V5, variable regions; FP, fusion peptide; NHR/CHR, N-/C-terminal heptad repeat; MPER, membrane-proximal extemal region; TM, transmembrane domain; KE, Kennedy epitope. Average of two replicate experiments (averaged on a linear scale before converting to log base 2).

FIG. 2. Assessment of data reproducibility. (A-C) Env libraries were sorted twice for high binding signals to (A) soluble CD4, (B) VRC01, and (C) PG16. Log₂ enrichment ratios from the two replicates are plotted, and qualitatively agree. Rarer mutations in the libraries (frequency <6×10⁻⁵) are grey. Mutations with a frequency <6×10⁶ are considered absent from the naïve libraries and are not plotted. R² values are calculated for mutations in black (frequency>6×10⁻⁵ in the naïve libraries). (D-F) Conservation scores for each residue were calculated by averaging the log₂ enrichment ratios for all substitutions at that position. Conservation scores for libraries sorted for binding (D) soluble CD4, (E) VRC01, and (F) PG16 show agreement between replicate experiments.

FIG. 3. Mapping conserved sites for ligand binding to Env structure. (A-C) Conservation scores from selecting Env libraries for binding (A) soluble CD4, (B) VRC01, or (C) PG16 are mapped to the surface of an Env protomer, from ≤−2 (conserved, black) to ≥0 (variable or under selection for change, white). The second and third protomers in the trimeric spike are shown as dark and pale grey ribbons. The binding sites for CD4, VRC01, and PG16 are shown with dashed circles. The model of BaL Env in the closed state was generated by sequence threading to PDB 5FYK, followed by loop building, and side chain and backbone minimization. (D) Differences between conservation scores for binding soluble CD4 and VRC01 are plotted from −2 (more conserved for CD4 binding, white) to +2 (more conserved for VRC01 binding, black) on the surface of an Env protomer oriented as above. (E) CD4-PG16 difference conservation plot colored from −2 (more conserved for CD4 binding, white) to +2 (more conserved for PG16 binding, black). (F) VRC01-PG16 difference conservation plot colored from −2 (more conserved for VRC01 binding, white) to +2 (more conserved for PG16 binding, black). (G) As in (E), but now plotting the CD4-PG16 difference conservation scores to a modeled structure of Env_{BaL} in the open CD4-bound conformation (based on PDB 5VN3). A single CD4 domain D1 is shown as a black ribbon.

FIG. 4. Env_{BaL} residues that physically contact CD4 are conserved for CD4 binding, while VRC01 and PG16 interactions are tolerant of Env sequence diversity. (A-E) Structural features of the CD4-gp120 interface are shown alongside heatmaps of the experimental enrichment ratios for CD4 binding. Log₂ enrichment ratios are plotted from ≤−3 (depleted, black) to ≥+1 (neutral/enriched, white). *, stop codons. The structural model was generated by threading the BaL Env sequence on to PDB 1GC1 with side chain-backbone minimization. CD4 is grey, and Env is colored by conservation score (most conserved residues are black). (F-H) Heatmaps of the experimental enrichment ratios from Env selection for VRC01 binding are shown alongside regions of the Env-VRC01 interface. The structural model was generated from PDB 5FYK as a template. VRC01 is grey and labeled according to Kabat numbering. In these panels, only Env-D279 (F) is highly conserved for VRC01 binding. (l-J) Heatmaps of log₂ enrichment ratios from the evolution of Env for PG16 binding are shown alongside a PG16-Env_{BaL} structural model (generated from PDB 4DQO as the template). PG16 is grey, and Env is colored by conservation score (most conserved residues are black). Env-N160, a site of glycosylation, is highly conserved for PG16 binding, whereas many other Env substitutions are tolerated.

FIG. 5. Neutralization of the electropositive apical cavity stabilizes Env in a PG16-recognized conformation. From the mutational scan, substitutions were identified that were both predicted to enhance PG16 binding, and were localized on subunit surfaces not expected to be major sites of direct bNAb interactions. The substitutions validated to enhance PG16 binding signal when expressed on Expi293F cells clustered to five sites, shown on a structural model of closed Env_{BaL}. PG16 is shown as a black cartoon, interacting glycans are white sticks, two Env protomers are shown as dark and pale grey surfaces, and the third Env protomer is shown as a pale grey ribbon. Mutations are indicated in the magnified insets. In the close-up of site 1 at left, positive electrostatic potential on the surface of two Env protomers is shown in black.

FIG. 6. Reduced PG16 binding to cells expressing Env variants that were depleted in the sequence-activity landscape. Twenty representative mutations were chosen that were depleted following FACS-based selection for PG16 binding in both replicate experiments. All 20 mutants were found to have reduced PG16 (2 nM) binding signal by flow cytometry when transfected in to Expi293F cells. Shown are histograms from one of two replicates. Wildtype is black, mutants are various shades of grey. The percent positive cells from both replicates is tabulated in the legend. Loss of PG16 binding signal may be due to reduced antibody affinity, incorrect folding, or decreased surface expression.

FIG. 7. (A-C) Engineered QES variants of (A) BaL, (B) Q769.d22, and (C) Q842.d12 Env show enhanced PG16 binding by flow cytometry when expressed on Expi293F cells. Wildtype proteins are grey, QES mutants are black. (n=3, mean±SD). (D-F) Binding of soluble CD4 to QES mutants of (D) BaL, (E) Q769.d22, and (F) Q842.d12 Env expressed on Expi293F cells. (n=3, mean±SD).

FIG. 8. Increased PG16 binding to Env mutated at the subunit interfaces is not mediated by changes in furin-dependent cleavage. (A) Polyclonal anti-Env blot of lysates from cells expressing BaL gp160 variants. (B) Cells were co-transfected with plasmids driving BaL gp160 and furin over-expression, and PG16 binding was measured by flow cytometry. (n=3, mean±SD).

FIG. 9. Mutations at the Env subunit interfaces that stabilize the PG16-recognized closed state do not prevent exposure of V3 region epitopes. Expi293F cells expressing wildtype BaL (light grey), BaL-QES.i01 (dark grey), or BaL-QES.i02 gp160 (black) were incubated with V3 region MAbs (A) 2442, (B) 268-D IV, (C) 39F, and (D) 3074. Bound antibody was detected with APC-conjugated anti-human IgG and flow cytometry. (n=3, mean±SD).

FIG. 10. Env-QES variants are competent for membrane fusion. (A) Cells expressing the CD4 and CCR5 receptors were co-incubated with cells expressing wildtype BaL, or BaL-QES.01 Env. The cytoplasm was stained with calcein and nuclei were stained with Hoechst 33342. Fused syncytia were observed as enlarged cells. (B) Enlarged/fused cells were quantified by flow cytometry after co-incubating receptor- and Env-expressing cells. (n=10, mean±SEM, Student's two-tailed unpaired t-test).

FIG. 11. Mutations within the Env core for increased PG16 binding. (A) A combinatorial library of surface-displayed gp140_{BaL} was sorted for high PG16 binding. Wildtype sequence is at top, with core mutations present in the library listed below each residue position. An alignment of 7 enriched clones/sequences with higher PG16 binding is shown, with the consensus in bold. (B) PG16 binding titration curves based on flow cytometry analysis of cells expressing surface-displayed wildtype (light grey), QES.i01 (dark grey), QES.i02 (black) or clone-27 (medium grey) gp140_{BaL}. n=4, mean±SD. (C) Substitutions found in clone-27 decrease PG16 binding to cells expressing full-length gp160_{BaL}, even when combined with QES.i01 (grey with dark grey outline) and QES.i02 mutations (grey with black outline). Data are mean (n=2), with error bars showing the range. (D) Local structure of Env_{BaL} surrounding buried residues I181, V254 and V255. Individual mutations found to enhance PG16 binding in full-length Env are shown in black. A predicted hydrogen bond from V254T to the backbone carbonyl of L261 is shown with a dashed line. An apolar cavity filled by V255M in the closed state becomes absent in the CD4-bound open conformation, in which only a single ‘bent’ rotamer of methionine can sterically fit.

FIG. 12. Mutation V255M within the Env core destabilizes the CD4-bound open conformation and reduces exposure of V3 region epitopes. (A) Cells expressing QES variants containing mutations to subunit surfaces (dark grey), or additional mutations to core residues (black), bind more PG16 than wildtype Env (pale grey). Env variants were tested from five HIV-1 strains (from top to bottom: BaL, Q769.d22, Q842.d12PG16, 25711, and DU422). Data are mean±SD, n=3-4. (B) Binding of CD4(D1-D2) to cells expressing Env variants. Inclusion of the V255M core mutation in constructs BaL-QES.i01.c01, Q769.d22-QES.i03.V255M, and DU422-QES.c03, reduces CD4 binding. Data are mean (n=2), with error bars showing the range. (C) Binding of V3-targeting antibodies (from top to bottom: monoclonal 2442, 268-D IV, 39F, and 3074) to cells expressing wildtype (grey) or QES.i01.c01 (black) BaL Env. Data are mean (n=2), with error bars showing the range.

FIG. 13. Mutations that stabilize the PG16-recognized conformation of Env also increase binding to PGT121 targeting the N332 glycan supersite. Binding of PGT121 to transfected cells expressing wildtype (grey) or QES variant (black) Env sequences from five HIV-1 strains: (A) BaL, (B) Q769.d22. (C) Q842.d12, (D) 25711, and (E) DU422). Data are mean (n=2), with error bars showing the range.

FIG. 14. Mutations that stabilize the PG16-recognized conformation of Env also increase binding to PGT128 targeting the N332 glycan supersite. Binding of PGT128 to transfected cells expressing wildtype (grey) or QES variant (black) Env sequences from five HIV-1 strains: (A) BaL, (B) Q769.d22. (C) Q842.d12, (D) 25711, and (E) DU422). Data are mean (n=2), with error bars showing the range.

FIG. 15. Mutations that stabilize the PG16-recognized conformation of Env may increase or decrease binding to PGT145 targeting the apical cavity. Binding of PGT145 to transfected cells expressing wildtype (grey) or QES variant (black) Env sequences from five HIV-1 strains: (A) BaL, (B) Q769.d22, (C) Q842.d12, (D) 25711, and (E) DU422). Data are mean (n=2), with error bars showing the range.

FIG. 16. Env-QES variants can catalyze membrane fusion. The formation of fused syncytia was measured when full-length Env-expressing and CD4/CCR5-expressing cells were co-incubated. QES mutations stabilizing the closed state do not significantly decrease cell fusion, but mutation V255M that destabilizes the CD4-bound open state does inhibit fusion (compare BaL-QES.i01.c01 to BaL-QES.i01, Q769.d22-QES.i03.V255M to Q769.d22-QES.i03, and DU422-QES.c03 to DU422 wildtype). Mean±SEM from n=10 replicates.

FIG. 17. Purified Env subunits from the BaL, Q769.d22, and Q842.d12 strains containing QES mutations are not shifted towards higher molecular weight forms. (A) Purified 8his-tagged BaL gp140 was separated by SEC on a Superose 6 10/300 column. In the absence of SOSIP mutations, the BaL-QES variants (dark grey and black) are indistinguishable from wildtype (light grey). (B) Purified 8his-tagged BaL gp120 was run on a Superose 6 10/300 gel filtration column. BaL-QES.i01 and BaL-QES.i02 share the same gp120 sequence. (C) SEC traces of purified wildtype Q769.d22 and Q769-QES.03 gp120. (D) SEC traces of purified wildtype Q842.d12 and Q842-QES.04 gp120.

FIG. 18. QES mutations stabilize a BG505 SOSIP trimer recognized by PG16. (A) His-tagged wildtype (grey) and QES.i03.c03 (black) BG505 SOSIP.664 were purified by nickel affinity chromatography and separated by SEC on a Superose 6 10/300 column. The trimer peak at left was identified based on a near identical elution volume to purified soluble gp140_{BaL} (FIG. 17A) and high affinity binding to PG16. (B) Coomassie-stained SDS electrophoresis gel of purified BG505 SOSIP.664 proteins. (C) BG505-QES.i03.c03 (black) binds higher levels of PG16 by ELISA than wildtype protein (grey). Mean±SD, n=4. (D) By ELISA, purified wildtype (grey) and QES.i03.c03 (black) BG505 SOSIP.664 bind VRC01 the same. Data are mean (n=2), with error bars showing the range. (E) Binding of tetrameric CD4-IgG2 to wildtype (grey; n=4) and QES.i03.c03 (blue; n=3) BG505 SOSIP.664 by ELISA. Mean±SD.

FIG. 19. Sequence-activity landscapes of gp140_{DU422} for interacting with the host CD4 receptor and bNAb PG16. (A) Three gp140_{DU422} SSM libraries were separately expressed in Expi293F cells and sorted for binding to sCD4. Log₂ enrichment ratios were calculated by comparing mutation frequencies in the sorted cells to the naïve libraries, and are plotted from ≤−2 (i.e. depleted, black) to ≥+3 (i.e. enriched, white). The primary structure of gp140 is on the horizontal axis, and amino acid substitutions are on the vertical axis. *, stop codons. In the upper schematic, gp120 and gp41 are dark and pale grey, respectively, the cleavage site is indicated with an arrow head, and notable regions are shaded black. (B) The sequence-activity landscape of gp140_{DU422} under FACS-based selection for binding to PG16.

FIG. 20. Correlations between replicate experiments. (A-C) FACS-based selections for sCD4 binding were repeated twice. Agreement between the replicate log₂ enrichment ratios for each gp140_{DU422} mutation are plotted for the (A) NT, (B) central, and (C) CT libraries. (D-F) Agreement between the residue conservation scores from replicate selections of the (A) NT, (B) central, and (C) CT libraries for sCD4 binding. (G-I) Log₂ enrichment ratios for every single amino acid substitution of gp140_{DU422} are plotted for two independent experiments where the (G) NT, (H) central, and (I) CT SSM libraries were selected for PG16 binding. (J-L) Agreement between the residue conservation scores from replicate selections of the (A) NT, (B) central, and (C) CT libraries for PG16 binding.

FIG. 21. Residues at the Env_{DU422}a trimer interface are more conserved for PG16 binding than for CD4 interactions. (A) Cartoon structure of the gp120 (dark grey) and gp41 (pale grey) protomer in the closed conformation (based on PDB 5FYK as). V1, V2, and V3 (shown as various shades of grey) form the apical trimerization domain. (B) An atomic model of trimeric DU422 gp140 in the closed conformation, with one protomer shown as a surface, and the other protomers shown as grey ribbons. The PG16-CD4 conservation difference scores are mapped to the protomer surface in the same orientation as (A), with black indicating residues preferentially conserved for PG16 binding, and white indicating residues more conserved for CD4 binding. On the right is a cutaway through the protomer surface, showing that preferential conservation for PG16 binding extends into the core of the trimerization domain. (C) Cryo-EM structure of gp120 (dark grey) and gp41 (pale grey) from a single protomer in the CD4-bound open conformation (PDB 5VN3¹³). Electron density was absent for V1 and V2 regions. (D) A model of DU422 gp140 in the open state bound to CD4 (black ribbon; CD4 is shown bound to only a single protomer). Colored as described in (B). The orientation of the protomer shown as a surface matches the orientation in (C).

FIG. 22. Increased hydrophobic packing at the gp120 inner-outer domain interface enhances expression of a PG16-recognized conformation. (A) Expi293F cells were transfected with the indicated gp160_{DU422} mutants, and binding of 2 nM PG16 (dark grey) or 50 nM sCD4 (light grey) was measured by flow cytometry. V181 L was previously shown to increase PG16 binding. Mean±SD, n=3. (B) Structures of DU422 Env (gp120, pale grey; gp41, dark grey) were modeled in closed and CD4-bound open conformations. Mutations are indicated in black.

### SUMMARY

An embodiment provides an HIV-1 Env protein or fragment thereof comprising one or more of the amino acid mutations listed in Table 1, wherein the amino acids are numbered by HXB2 numbering.

Another embodiment provides an HIV-1 Env protein or fragment thereof comprising one or more of the sets of amino acid mutations listed in Table 2, wherein the amino acids are numbered by HXB2 numbering.

Even another embodiment provides an HIV-1 Env protein or fragment thereof comprising one or more of the amino acid mutations listed in Table 1 or Table 2, wherein the protein or fragment thereof has at least one mutation shown in Tables 1 or 2 and otherwise has about 95% or more sequence identity to an HIV-1 Env protein (such as a wild-type HIV-1 Env protein).

Yet another embodiment provides a trimeric complex or portion thereof comprising HIV-1 Env proteins or fragments comprising one or more of the mutations listed in Table and Table 2 in a trimeric conformation.

Still another embodiment provides an immunogen comprising an HIV-1 Env protein or fragment thereof comprising one or more of the amino acid mutations listed in Table 1 or Table 2 or an HIV-1 Env trimeric complex or portions thereof comprising one or more of the amino acid mutations listed in Table 1 or Table 2.

Even another embodiment provides a method of screening a compound for binding to one or more proteins thereof, wherein the one or more proteins comprise an HIV-1 Env protein or fragment thereof comprising one or more of the amino acid mutations listed in Table 1 or Table 2, a trimeric complex or portions thereof comprising one or more of the amino acid mutations listed in Table 1 or Table 2, or combinations thereof. The method comprises providing the one or more proteins, fragments, complexes or portions thereof; contacting the one or more proteins, fragments, complexes or portions thereof with the compound; and determining the ability of the compound to bind to the one or more proteins, fragments, complexes or portions. The one or more proteins, fragments, trimeric complexes, or portions thereof can comprise 2, 5, 10, 15, or more proteins, fragments, trimeric complexes, or portions thereof. The compound can inhibit an HIV-mediated activity. The compound can be provided in a library.

An embodiment provides a library comprising two or more (e.g., 2, 5, 10, 20, 30, 50, 100 or more) HIV-1 Env proteins or fragments thereof comprising one or more of the amino acid mutations listed in Table 1 or Table 2 or an HIV-1 Env trimeric complex or portions thereof comprising one or more of the amino acid mutations listed in Table 1 or Table 2.

Another embodiment provides a nucleic acid molecule encoding an HIV-1 Env protein or fragment thereof comprising one or more of the amino acid mutations listed in Table 1 or Table 2 or a trimeric complex or portions thereof comprising one or more of the amino acid mutations listed in Table 1 or Table 2.

Still another embodiment provides a vector comprising a nucleic acid molecule described herein.

Yet another embodiment provides a host cell comprising a vector described herein.

Even another embodiment provides a method of producing an HIV-1 Env protein, fragment thereof, HIV-1 Env trimeric complex, or portion thereof comprising culturing a host cell described herein in a culture medium to produce the protein, fragment, complex, or portion thereof. The host cell can be a mammalian cell having the ability to glycosylate proteins.

An embodiment provides a composition comprising one or more HIV-1 Env proteins or fragments thereof described herein, one or more HIV-1 Env trimeric complexes or portions thereof described herein, and a pharmaceutically acceptable carrier. The composition can comprise one or more HIV-1 Env proteins or fragments thereof or one or more HIV-1 Env trimeric complexes or portions thereof. The composition can also comprise one or more HIV-1 Env proteins or fragments thereof and one or more HIV-1 Env trimeric complexes or portions thereof. The composition can further comprise an adjuvant.

Even another embodiment provides method for eliciting an immune response against an HIV-1 infected cell in a subject comprising administering to the subject an amount of an HIV-1 Env protein, fragment thereof, HIV-1 Env trimeric complex or portion thereof described herein, effective to elicit an immune response in the subject.

Yet another embodiment provides a method for preventing a subject from becoming infected with HIV-1 comprising administering to the subject a prophylactically effective amount of an amount of an HIV-1 Env protein, fragment thereof. HIV-1 Env trimeric complex or portion thereof described herein, such that the subject is prevented from becoming infected with HIV-1.

Still another embodiment provides a method for reducing the likelihood of a subject becoming infected with HIV-1 comprising administering to the subject an amount of an HIV-1 Env protein, fragment thereof, HIV-1 Env trimeric complex or portion thereof described herein, effective to reduce the likelihood of the subject becoming infected with HIV-1. The subject may have been exposed to HIV-1.

Another embodiment provides a method for delaying the onset of, or slowing the rate of progression of, an HIV-1-related disease or symptom in an HIV-1-infected subject comprising administering to the subject an amount of an amount of an HIV-1 Env protein, fragment thereof, HIV-1 Env trimeric complex, or portion thereof described herein, effective to delay the onset of, or slow the rate of progression of the HIV-1-related disease or symptom in the subject.

An embodiment provides a method of isolating antibodies that specifically bind to an HIV-1 Env protein, fragment thereof, HIV-1 Env trimeric complex, or portion thereof described herein. The method comprises administering an effective amount of an HIV-1 Env trimeric complex or portion thereof, an HIV-1 Env protein or fragment thereof, an HIV-1 Env nucleic acid molecule, a vector, a host cell or a pharmaceutical composition described herein to a subject to generate antibodies that specifically bind to an HIV-1 Env protein or HIV-1 Env trimeric complex; and isolating the antibodies.

Another embodiment provides a method of identifying antibodies that specifically bind to an HIV-1 Env protein, fragment thereof, HIV-1 Env trimeric complex, or portion thereof as described herein. The method comprises administering an effective amount of an immunogen selected from: an HIV-1 Env trimeric complex or portion thereof, an HIV-1 Env protein or fragment thereof, an HIV-1 Env nucleic acid molecule, a vector, a host cell or a pharmaceutical composition described herein to B cells in an in vitro cell culture system to generate antibodies that specifically bind to the HIV-1 Env protein, fragment thereof, HIV-1 Env trimeric complex or portion thereof. Antibodies specific for the administered complex or composition are then isolated.

Yet another embodiment provides a method of making an isolated hybridoma that produces a broadly neutralizing antibody that specifically binds to an HIV-1 Env trimeric complex, portion thereof, an HIV-1 Env protein, or fragment thereof. The method comprises immunizing a mammal with an effective amount of: an HIV-1 Env trimeric complex or portion thereof, an HIV-1 Env protein or fragment thereof, an HIV-1 Env nucleic acid molecule, a vector, a host cell or a pharmaceutical composition described herein. Splenocytes are isolated from the immunized mammal and the isolated splenocytes are fused with an immortalized cell line to form hybridomas. Individual hybridomas are screened for production of an antibody that specifically binds with the HIV-1 Env protein or fragment thereof or HIV-1 Env trimeric complex or portion thereof to isolate the hybridoma.

Still another embodiment provides a method of producing a stable HIV-1 Env trimer in a closed conformation. The method comprises making one or more of the amino acid mutations or sets of mutations described herein in an HIV-1 Env protein and expressing the protein such that a stable HIV-1 Env trimer in a closed conformation is produced.

HIV-1 infection is initiated by viral Env engaging the host receptor CD4, triggering Env to transition from a ‘closed’ to ‘open’ conformation during the early events leading to virus-cell membrane fusion. Using deep mutational scanning, sequence-activity landscapes were defined for Env interacting with CD4, antibody VRC01 that binds the CD4 site but fails to induce the open state, and antibody PG16 that recognizes closed Env. Compared to CD4 or VRC01 binding, the Env trimer interface was under selection for PG16 recognition, and mutations that enhance presentation of the PG16 quatemary epitope frequently reduce positive charge at the Env apex, suggesting this region is primed for opening by electrostatic repulsion. Positive stabilization of the closed state is insufficient to reduce CD4 binding, membrane fusion, or V3 epitope exposure, which instead requires explicit destabilization of the open conformation. Mutations described herein stabilize a closed Env conformation, are broadly applicable to different HIV-1 strains, and can assist in the engineering of Env-based immunogens that better present epitopes recognized by broadly neutralizing antibodies.

Env sequence preferences are determined independent of infection and virus propagation for interactions with three protein ligands that act as conformational probes: CD4, which induces the open Env conformation and binds monomeric gp120 with highest affinity; bNAb VRC01, which binds tightly to both monomeric gp120 and mature Env without inducing the open conformation; and bNAb PG16, which exclusively binds closed trimeric Env^{1,38-44}. While soluble SOSIP proteins have been extensively engineered, less focus has been applied to conformational stabilization of full-length Env that may be better suited for virus-like particle or DNA vaccines. Mutations are provided herein that stabilize the PG16 quatemary epitope in full-length Env sequences from representative strains in clades A, B and C, while simultaneously enhancing presentation of epitopes recognized by PGT121 and PGT128 bNAbs, and also revealing an electrostatic repulsion mechanism for inducing the closed-to-open transition. It would be ideal if there were a suite of mutations for applying broadly to any HIV-1 strain, which stabilize Env in a closed trimer for improved bNAb elicitation. A HIV-1 vaccine could then be rapidly modified and updated to contain stabilized Env sequences from local prevailing strains. The HIV-1 Env mutants described herein address a pressing need in that they can be rapidly used as a focused mutational screen to conformationally stabilize Env from diverse HIV-1 strains.

### DETAILED DESCRIPTION

Alignments of protein sequences observed in nature are very effective at revealing conserved residues in primary structure for correct folding and function. However, a natural sequence like HIV-1 Env is shaped by multiple activities; Env must fold, traffic to the cell surface, be incorporated into a budding virion, bind target receptors, mediate membrane fusion, and escape antibody neutralization. Here, in vitro selection is used to focus on the specific activities of folding to a trimeric closed conformation and binding to the CD4 receptor. Sequence-activity landscapes provide insight into Env mutational tolerance for acquiring closed and CD4-bound conformations, information which is not at all obvious from a multiple sequence alignment. This information provides mechanistic insights, and is leveraged for engineering full-length Env with properties that may prove especially useful for vaccines that incorporate membrane-anchored Env (e.g. virus-like particles or DNA vaccines), and can also be applied to soluble extracellular constructs like SOSIP.

Increased presentation of the PG16-recognized conformation can enhance fusion to target membranes, consistent with the closed state being relevant to the molecular events of infection and an excellent target for neutralization by vaccines. Env-QES.i01 and QES.i02 variants still bind CD4 and expose V3 region epitopes, either because of dynamic or induced conformational fluctuations, or persistent conformational heterogeneity that includes trimeric, monomeric, closed, open and/or misfolded forms. V3 region epitopes are hidden only by inclusion of an additional core mutation (V255M) to destabilize the CD4-bound open state, and some of the mutations identified herein from the mutational scan of DU422 gp140 (e.g. Y484W) may similarly act to destabilize the CD4-bound open state. Likewise, trimer-stabilized SOSIP gp140 constructs also have persistent binding to V3 antibodies^{17,20}, and induce undesirable V3 non-neutralizing responses in immunized animals^{16,17}. This has necessitated negative selection strategies to explicitly reduce V3 loop exposure^{17,24}. The findings described herein emphasize that positive stabilization alone appears insufficient to prevent Env opening, and the open state must be explicitly destabilized.

### HIV-1 Env Protein Mutations

HIV-1 is completely dependent upon the Env protein to enter cells. HIV-1 Env is formed by a homotrimeric complex of gp160 subunits that are cleaved by host proteases during maturation into extracellular gp120 and membrane-tethered gp41, which remain non-covalently associated in a ‘closed’ conformation. Production of Env trimeric complexes that mimic the native spike, however, is challenging in part because the recombinant trimers either are unstable or aggregate. Therefore, mutants of Env proteins that can form stable trimeric complexes in the closed conformation are desirable. Membrane distal and membrane proximal aspects of the HIV-1 trimer in the closed conformation include several distinct structural elements that are absent from the corresponding regions of the HIV-1 Env trimer in its CD4-bound open conformation. The following mutations can be used to generate mutant, stable Env trimeric complexes in the closed conformation across many clades of HIV-1. For example, the amino acid substitutions disclosed herein can be used to alter the Env protein in any clades or subtypes of Group M, O, N, or P HIV-1 strains.

HIV-1 Env proteins and nucleic acid sequences encoding Env proteins and methods for the manipulation and insertion of such nucleic acid sequences into vectors, are well known (see, e.g., HIV Sequence Compendium, Division of AIDS. National Institute of Allergy and Infectious Diseases (2013); HIV Sequence Database (hiv-web.lanl.gov/content/hiv-db/mainpage.html); Sambrook et al. (Molecular Cloning: A Laboratory Manual, 4^{th} ed, Cold Spring Harbor, N.Y., 2012); Ausubel at al. (In Current Protocols in Molecular Biology, John Wiley & Sons, New York, through supplement 104, 2013). HIV-1 Env protein sequences are known and are available in the HIV Sequence Database (hiv-web.lanl.gov/content/hiv-db/mainpage.html).

Amino acid mutations or substitutions are described herein. An amino acid substitution is the replacement of one amino acid in a polypeptide with a different amino acid or with no amino acid (i.e., a deletion).

An embodiment provides an HIV-1 Env protein or fragment thereof comprising one or more of the amino acid mutations (e.g., 1, 2, 3, 4, 5, 6, 7, 8, 9, 10, or more mutations) in Table A. A fragment can be about 10, 20, 30, 40, 50, 75, 100, 200, 300, 400, 500, 600, or more amino acids as long as it contains an amino acid mutation listed in Tables A-H and can induce an immunogenic response in a subject (e.g., a mammal such as a human). The amino acids are numbered by HXB2 numbering.

TABLE A

T49D
Q114A
K117V
K117Y
P124D
T163D
R166E
R166F
R166L
I181L
V181L
V200E
V200T
A200E
V208M
F223Y
V254T
V255M
T283P
R315A
F382W
R315Q
K315A
R432T
Y484W
G514P
G516Q
R557Q
L581D
I595M
L663N

An embodiment provides an HIV-1 Env protein or fragment thereof comprising one or more of the combination of amino acid mutations (e.g., 1, 2, 3, 4, 5, or more sets of mutations) in Table B. The amino acids are numbered by HXB2 numbering.

TABLE B
V200E + F223Y
R432T + R557Q
R315A + L663N
Q114A + V200T
K117V + T163D
K117V + R315A
Q114A + L663N
V200T + I595M
T49D + R315A + I595M
R166L + F223Y + L663N
K117V + R166L + R315A
K117V + R166L + F223Y
T163D + V200T + L581D
R166L + R315A + G514P
R315A + L663N + T49D
R315A + L663N + R166L
R315A + L663N + F223Y
R315A + L663N + R432T
R315A + L663N + I595M
T49D + P124D + I595M (QES.i01)
P124D + L663N
T49D + R315A + I595M + K117Y
T49D + R315A + I595M + R166L
T49D + R315A + I595M + L663N
T49D + K117V + R315A
K117V + R315A + L663N
K117V + R166L + F223Y + I595M
K117V + R166L + F223Y + L663N
P124D + R315A
P124D + R315A + L663N
T49D + R315A + I595M + L663N
T49D + P124D + R315A + I595M + L663N
T49D + P124D + R315A + I595M
T49D + P124D + I595M + L663N
T49D + P124D + R315A + G514P + I595M
T49D + P124D + L663N (QES.i02)
I181L + V254T (QES.c02)
I181L + V255M
V254T + V255M
T49D + P124D + I595M + I181L
T49D + P124D + I595M + V254T
T49D + P124D + I595M + I181L + V255M (QES.i01.c01)
V181L + V254T
V181L + V255M
V181L + V254T + V255M (QES.c03)
P124D + I595M
P124D + F223Y + I595M
A200E + F223Y + I595M (QES.i03)
P124D + R557Q
R557Q + F223Y
P124D + A200E + F223Y + I595M
A200E + F223Y + R557Q + I595M
R166L + A200E
R166L + R557Q
R166L + R557Q + I595M
A200E + R557Q + I595M
P124D + A200E
P124D + R166L
P124D + R557Q + I595M
P124D + R166L + R557Q
R166L + A200E + R557Q
R166L + F223Y + R557Q
P124D + R166L + R557Q + I595M
P124D + F223Y + R5570 + I595M (QES.i04)
I181L + V254T + V255M
P124D + F223Y + R557Q + I595M + I181L (QESi04.I181L)
P124D + F223Y + R557Q + I595M + I181L + V255M
K117Y + A200E
T163D + A200E
A200E + L581D
A200E + R557Q
A200E + I595M
V181L + A200E
K117Y + L581D
A200E + F223Y + R557Q
A200E + L581D + I595M
A200E + L581D + I595M + L663N
K117Y + L581D + I595M
K117Y + L581D + I595M + L663N
A200E + F223Y + I595M + V181L, and
A200E + F223Y + I595M + V181L + V255M (QES.i03.c01)

An embodiment provides an HIV-1 Env_{BaL} protein or fragment thereof comprising one or more of the amino acid mutations (e.g., 1, 2, 3, 4, 5, or more mutations or 1, 2, 3, 4, 5, or more sets of mutations) in Table C. The amino acids are numbered by HXB2 numbering.

TABLE C

T49D
Q114A
K117V
K117Y
P124D
T163D
R166E
R166F
R166L
V200E
V200T
F223Y
R315A
R315Q
R432T
G514P
G516Q
R557Q
L581D
I595M
L663N
V200E + F223Y
R432T + R557Q
R315A + L663N
Q114A + V200T
K117V + T163D
K117V + R315A
Q114A + L663N
V200T + I595M
T49D + R315A + I595M
R166L + F223Y + L663N
K117V + R166L + R315A
K117V + R166L + F223Y
T163D + V200T + L581D
R166L + R315A + G514P
R315A + L663N + T49D
R315A + L663N + R166L
R315A + L663N + F223Y
R315A + L663N + R432T
R315A + L663N + I595M
T49D + P124D + I595M (BaL-QES.i01)
P124D + L663N
T49D + R315A + I595M + K117Y
T49D + R315A + I595M + R166L
T49D + R315A + I595M + L663N
T49D + K117V + R315A
K117V + R315A + L663N
K117V + R166L + F223Y + I595M
K117V + R166L + F223Y + L663N
P124D + R315A
P124D + R315A + L663N
T49D + R315A + I595M + L663N
T49D + P124D + R315A + I595M + L663N
T49D + P124D + R315A + I595M
T49D + P124D + I595M + L663N
T49D + P124D + R315A + G514P + I595M
T49D + P124D + L663N (BaL-QES.i02)
I181L
V254T
V255M
I181L + V254T
I181L + V255M
V254T + V255M
T49D + P124D + I595M + I181L
T49D + P124D + I595M + V254T
T49D + P124D + I595M + I181L + V255M
(QES.i01.c01)

An embodiment provides an HIV-1 Env_{DU422} protein or fragment thereof comprising one or more of the amino acid mutations (e.g., 1, 2, 3, 4, 5, or more mutations or 1, 2, 3, 4, 5, or more sets of mutations) in Table D. The amino acids are numbered by HXB2 numbering.

TABLE D

Q114A
V181L
V208M
V255M
T283P
F382W
Y484W
V254T + V255M
V181L + V254T
V181L + V255M
V181L + V254T + V255M (QES.c03)

An embodiment provides an HIV-1 Env_{Q769.d22} protein or fragment thereof comprising one or more of the amino acid mutations (e.g., 1, 2, 3, 4, 5, or more mutations or 1, 2, 3, 4, 5, or more sets of mutations) in Table E. The amino acids are numbered by HXB2 numbering.

TABLE E

P124D
A200E
F223Y
K315A
R557Q
I595M
L663N
P124D + I595M
P124D + F223Y + I595M
A200E + F223Y + I595M (Q769-QES.i03)
P124D + R557Q
R557Q + F223Y
P124D + A200E + F223Y + I595M
A200E + F223Y + R557Q + I595M
V255M
V254T + V255M
A200E + F223Y + I595M + V255M
(QES.i03.V255M)

An embodiment provides an HIV-1 Env Q842.d12 protein or fragment thereof comprising one or more of the amino acid mutations (e.g., 1, 2, 3, 4, 5, or more mutations or 1, 2, 3, 4, 5, or more sets of mutations) in Table F. The amino acids are numbered by HXB2 numbering.

TABLE F
P124D
R166L
A200E
R557Q
I595M
R166L + A200E
R166L + R557Q
R166L + R557Q + I595M
A200E + R557Q + I595M
P124D + A200E
P124D + R166L
P124D + R557Q + I595M
P124D + R166L + R557Q
R166L + A200E + R557Q
R166L + F223Y + R557Q
P124D + R166L + R557Q + I595M
P124D + F223Y + R557Q + I595M (Q842-QES.i04)
I181L
V255M
V254T + V255M
I181L + V255M
I181L + V254T + V255M
P124D + F223Y + R557Q + I595M + I181L (QES.i04.I181L)
P124D + F223Y + R557Q + I595M + I181L + V255M

An embodiment provides an HIV-1 Env 25711 protein or fragment thereof comprising one or more of the amino acid mutations (e.g., 1, 2, 3, 4, 5, or more mutations or 1, 2, 3, 4, 5, or more sets of mutations) in Table G. The amino acids are numbered by HXB2 numbering.

TABLE G

I181L
I181L + V254T (QES.c02)
I181L + V255M
I181L + V254T + V255M

An embodiment provides an HIV-1 BG505 SOSIP.664 protein or fragment thereof comprising one or more of the amino acid mutations (e.g., 1, 2, 3, 4, 5, or more mutations or 1, 2, 3, 4, 5, or more sets of mutations) in Table H. The amino acids are numbered by HXB2 numbering.

TABLE H

K117Y
T163D
V181L
A200E
F223Y
V255M
I595M
L663N
K117Y + A200E
T163D + A200E
A200E + L581D
A200E + R557Q
A200E + I595M
V181L + A200E
V181L + V255M
V254T + V255M
K117Y + L581D
A200E + F223Y + R557Q
A200E + L581D + I595M
A200E + F223Y + I595M (QES.i03)
A200E + L581D + I595M + L663N
K117Y + L581D + I595M
K117Y + L581D + I595M + L663N
A200E + F223Y + I595M + V181L
A200E + F223Y + I595M + V255M
A200E + F223Y + I595M + V181L + V255M
(QES.i03.c01)

An HIV-1 Env mutant protein described herein comprises one or more of the amino acid substitutions described herein (e.g., those shown in Tables A, B, C, D, E, F, G, or H). HIV-1 Env mutant proteins described herein can comprise the disclosed amino acid mutation or set of amino acid mutations and otherwise have 75, 80, 90, 95, 99% or more sequence identity to any HIV-1 Env protein (e.g., an HIV-1 Env_{BaL} protein, an HIV-1 Env_{DU422} protein, an HIV-1 Env_{Q769.d22} protein, an HIV-1 Env Q842.d12 protein, an HIV-1 Env 25711 protein or other HIV-1 Env proteins). In some examples, an amino acid in a polypeptide is substituted with an amino acid from a homologous polypeptide, for example, an amino acid in a recombinant Clade A HIV-1 Env polypeptide can be substituted with the corresponding amino acid from a Clade B HIV-1 Env polypeptide.

All amino acid numbering of HIV-1 Env used herein refers to HXB2 number. HXB2 numbering is described in detail in, for example, Korber et al. Numbering Positions in HIV Relative to HXB2CG, in Korber et al., eds., Human Retroviruses and AIDS 1998, pp. III-102-III-111, Los Alamos National Laboratory, Los Alamos, N. Mex., report LA-UR 99-1704, which is incorporated by reference in its entirety.

Korber presents a clearly numbered set of proteins, and the full length genome, for HIV HXB2, GenBank accession number K03455. HIV HXB2 is also known as: HXBc2, for HXB clone 2; HXB2R, and HXB2CG in GenBank, for HXB2 complete genome. The HXB2 Env sequence is shown in SEQ ID NO:1 below.

```
(SEQ ID NO: 1)
    1 mrvkekyqhl wrwgwrwgtm llgmlmicsa teklwvtvyy gvpvwkeatt tlfcasdaka
  61 ydtevhnvwa thacvptdpn pqevvlvnvt enfnmwkndm veqmhediis lwdqslkpcv
121 kltplcvslk ctdlkndtnt nsssgrmime kgeikncsfn istsirgkvq keyaffykld
181 iipidndtts ykltscntsv itqacpkvsf epipihycap agfailkcnn ktfngtgpct
241 nvstvqcthg irpvvstqll lngslaeeev virsvnftdn aktiivqlnt sveinctrpn
301 nntrkririq rgpgrafvti gkignmrqah cnisrakwnn tlkqiasklr eqfgnnktii
361 fkqssggdpe ivthsfncgg effycnstql fnstwfnstw stegsnnteg sdtitlpcri
421 kqiinmwqkv qkamyappis gqircssnit gllltrdggn snneseifrp gggdmrdnwr
481 selykykvvk ieplgvaptk akrrvvqrek ravgigalfl gflgaagstm gaasmtltvq
541 arqllsgivq qqnnllraie aqqhllqltv wgikqlqari laverylkdq qllgiwgcsg
601 klicttavpw naswsnksle qiwnhttwme wdreinnyts lihslieesq nqqekneqel
661 leldkwaslw nwfnitnwlw yiklfimivg glvqlrivfa vlsivnrvrq gysplsfqth
721 lptprgpdrp egieeegger drdrsirlvn gslaliwddl rslclfsyhr lrdlllivtr
781 ivellgrrgw ealkywwnll qywsqelkns aysllnatai avaegtdrvi evvqgacrai
841 rhiprrirqg lerill.
```

Therefore, the HIV-1 Env amino acid mutation “T49D” means that the T at position 49 of the Env sequence is changed to D. In some cases the first amino acid of the mutation abbreviation may not match precisely to the HXB2 numbering. HXB2 has a V at position 200, but one mutation described herein is A200E. That means the HIV strain studied has an A at the HXB2 200 position and that the A is mutated to E. One of skill in the art is well-versed in the HXB2 numbering system.

A protein is a polymer of amino acids covalently linked by amide bonds. A protein can be post-translationally modified. A purified protein is a protein preparation that is substantially free of cellular material, other types of proteins, chemical precursors, chemicals used in synthesis of the protein, or combinations thereof. A protein preparation that is substantially free of cellular material, culture medium, chemical precursors, chemicals used in synthesis of the protein, etc., has less than about 30%, 20%, 10%, 5%, 1% or more of other proteins, culture medium, chemical precursors, and/or other chemicals used in synthesis. Therefore, a purified protein is about 70%, 80%, 90%, 95%, 99% or more pure. A purified protein does not include unpurified or semi-purified cell extracts or mixtures of protein that are less than 70% pure.

The term “proteins” can refer to one or more of one type of protein (a set of proteins). “Proteins” can also refer to mixtures of two or more different types of proteins (a mixture of proteins). The terms “proteins” or “protein” can each also mean “one or more proteins.” As used in the specification, “proteins” refers to both full-length proteins and fragments of proteins. The HIV-1 Env proteins comprising one or more mutations described herein are non-naturally occurring.

HIV-1 Env proteins can be fragments of the proteins described herein. For example an HIV-Env can comprise a fragment of an HIV-1 Env protein, having one or more of the mutations described herein. A fragment can be about 20, 50, 75, 100, 125, 150, 175, 200, 225, 250, 275, 300, 325, 350, 375, 400, 425, 450, 500, 600, 700, 800 or more amino acids in length. A fragment can be about 800, 700, 600, 500, 450, 425, 400, 375, 350, 325, 300, 275, 250, 225, 200, 175, 150, 125, 100, 75, 50, 20, or less amino acids in length. In an embodiment, an HIV-1 Env protein has about 1, 5, 10, 20, 30, 40, 50 or more amino acids truncated from the C-terminus. Such truncations can advantageously remove motifs for internalization that reduce surface expression.

An HIV-1 Env protein or fragment thereof can be linked to an epitope or affinity tag such as polyhistidine, DYKDDD (SEQ ID NO:2) tag, c-myc tag, Strep tag, TAP tag, and HA tag.

A mutated protein comprises at least one deleted, inserted, and/or substituted amino acid, which can be accomplished via mutagenesis of polynucleotides encoding these amino acids. Mutagenesis includes well-known methods in the art, and includes, for example, site-directed mutagenesis by means of PCR or via oligonucleotide-mediated mutagenesis as described in Sambrook et al., Molecular Cloning-A Laboratory Manual, 2nd ed., Vol. 1-3 (1989).

A protein can include multiple polypeptide chains. For example, mature HIV-1 Env comprises gp120 and gp41 polypeptide chains. A single contiguous polypeptide chain of amino acid residues can include multiple polypeptides. For example, a single chain HIV-1 Env can comprise a gp120 polypeptide linked to a gp41 polypeptide by a peptide linker.

Proteins and polynucleotides have about 75, 80, 85, 90, 95, 96, 97, 98, 99% or more sequence identity to proteins and polynucleotides described herein (e.g., proteins have one or more of the mutations show in Table A and Table B) can be used herein. Proteins and polynucleotides that have about 75, 80, 85, 90, 95, 96, 97, 98, 99% or more sequence identity to polypeptides and polynucleotides described herein while retaining one or more of the mutations show in Table A and Table B can also be used herein.

Sequence identity is the similarity between amino acid sequences and is expressed in terms of similarity between sequences. Sequence identity can be measured in terms of percentage identity (or similarity or homology); the higher the percentage, the more similar the two sequences are. Homologs, orthologs, or variants of a protein or nucleic acid molecule will have a relatively high degree of sequence identity when aligned using standard methods.

Methods of alignment of sequences for comparison are well known in the art. Programs and alignment algorithms are described in, for example, Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, provides a detailed discussion of sequence alignment methods and homology calculations.

Two sequences can be aligned and the number of matches can be determined by counting the number of positions where an identical nucleotide or amino acid residue is present in both sequences. The percent sequence identity is calculated by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a peptide sequence that has 1776 matches when aligned with a test sequence having 2000 is 88.8 percent identical to the test sequence (1776/2000×100=88.8). The percent sequence identity value is rounded to the nearest tenth. For example, 88.11, 88.12, 88.13, and 88.14 are rounded down to 88.1, while 88.15, 88.16, 88.17, 88.18, and 88.19 are rounded up to 88.2. The length value will always be an integer.

Homologs and variants of a protein and nucleic acid molecule are typically characterized by possession of at least about 75%, for example at least about 75%, 80%, 85%, 90%, 91%, 92%, 93%, 94%, 95%, 96%, 97%, 98% or 99% sequence identity counted over the full length alignment with the amino acid or nucleic acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website.

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Natl. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Sambrook et al. (Molecular Cloning: A Laboratory Manual, 4^{th} ed, Cold Spring Harbor, N.Y., 2012) and Ausubel et al. (In Current Protocols in Molecular Biology, John Wiley & Sons, New York, through supplement 104, 2013). One example of a useful algorithm is PILEUP. PILEUP uses a simplification of the progressive alignment method of Feng & Doolittle, J. Mol. Evol. 35:351-360, 1987. The method used is similar to the method described by Higgins & Sharp, CABIOS 5:151-153. 1989. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nucl. Acids Res. 12:387-395, 1984).

Another example of algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information (ncbi.nlm.nih.gov). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915, 1989).

In an embodiment an HIV-1 Env protein is from strain/clade BaL. DU422, YU-2, 25711, Q769.d22. Q842.d12, BG505, 191084, AD8, B41, SF162P3, 001428, SHIV327C, Hu_A10, AC10, ZM197M, CH110, H031, CH111, 257-31, PVO, CH115, or any other HIV-1 strain or clade and comprises one or more of the mutations or sets of mutations described herein.

### Libraries

An embodiment provides a library comprising two or more (e.g., 2, 5, 20, 10, 50, 75, 100 or more) of the mutated HIV-1 Env proteins disclosed herein. Another embodiment provides a display library of one or more of the mutated HIV-1 Env proteins disclosed herein. The mutated HIV-1 Env proteins can comprise a fragment of the mutated HIV-1 Env protein or a full-length mutated HIV-1 Env protein. A fragment of the mutated HIV-1 Env protein comprises at least one of the disclosed mutations and is about 10, 20, 50, 100, 200, 300, 400, 500, 600, 700, 800, or more amino acids in length. In an embodiment, a fragment can induce an immune response.

A mutated HIV-1 Env protein or fragment thereof can be displayed on the surface of a library, by for example the C terminus. This is a display library.

A mutant HIV-1 Env display library can comprise a phage display library. A phage display library can be a collection of phage that has been genetically engineered to express one or more mutant HIV-1 Env proteins or fragments thereof on their outer surface. In an embodiment nucleic acid molecules encoding the mutant HIV-1 Env proteins or fragments thereof are inserted in frame into a gene encoding a phage capsule protein. In another embodiment, a phage display library is a collection of phage that displays one or more mutant HIV-1 Env proteins or fragments thereof on their outer surface.

A display library can be, for example, a phage display library, a phagemid display library, a virus display library, a bacterial cell display library, a mammalian cell display library, yeast display library, a λgt11 library, an in vitro library selection system (CIS display), an in vitro compartmentalization library, an antibody-ribosome-mRNA (ARM) ribosome display library, or a ribosome display library.

Methods of making and screening such display libraries are well known to those of skill in the art and described in, e.g., Molek et al. (2011) Molecules 16, 857-887; Boder et al., (1997) Nat Biotechnol 15, 553-557; Scott et al. (1990) Science 249, 386-390; Brisette et al. (2007) Methods Mol Biol 383, 203-213; Kenrick et al. (2010) Protein Eng Des Sel 23, 9-17; Freudl et al. (1986) J Mol Biol 188, 491-494; Getz et al. (2012) Methods Enzymol 503, 75-97; Smith et al. (2014) Curr Drug Discov Technol 11, 48-55; Hanes, et al. (1997) Proc Natl Acad Sci USA 94, 4937-4942; Lipovsek et al., (2004) J Imm Methods 290, 51-67; Ullman et al. (2011) Brief. Funct. Genomics, 10, 125-134; Odegrip et al. (2004) Proc Natl Acad Sci USA 101, 2806-2810; and Miller et al. (2006) Nat Methods 3, 561-570.

A mutant HIV-1 Env library or display library can be screened for biological activity.

### Trimeric Complexes

There is a need for stabilized HIV-1 Env trimeric complexes that have improved percentage of trimeric complex formation (e.g., about 10, 20, 30, 40, 50 60, 70% or more improved trimeric complex formation as compared to wild-type HIV-1 Env trimeric complex formation), improved trimeric complex yield (e.g., about 10, 20, 30, 40, 50 60, 70% or more improved trimeric complex yield as compared to wild-type HIV-1 trimeric complex yield), and/or improved trimeric complex stability (e.g., about 10, 20, 30, 40, 50 60, 70% or more better HIV-1 Env trimeric complex stability as compared to wild-type HIV-1 Env trimeric complex stability).

An HIV-1 Env trimeric complex comprises HIV-1 Env proteins comprising at least one mutation described in a mature trimeric conformation. Three HIV-1 Env proteins come together to form one HIV-1 Env trimeric complex Therefore, each HIV-1 Env trimeric complex has three gp120 subunits and three gp41 subunits. A portion of an HIV-1 Env trimeric complex can be about 100, 200, 300, 400, 500, 600, 700 or more amino acids, as long as it contains an amino acid mutation listed in Tables A-H, can induce an immunogenic response in a subject (e.g., a mammal such as a human), and includes at least a portion of three gp120 subunits and at least a portion of three gp41 subunits.

An HIV-1 Env trimeric complex can be a chimeric HIV-1 Env trimeric complex that comprises amino acid sequences from two or more different HIV-1 clades or amino acid sequences from two or more different mutant HIV-1 Env proteins.

HIV-1 trimeric complex formation can be measured by an antibody binding assay using antibodies that bind specifically to the trimeric form of the HIV-1 Env protein. Examples of trimeric complex specific antibodies that can be used to detect a trimer form include, but are not limited to, monoclonal antibodies (mAbs) PGT145, PGDM1400, PG16, and PGT151. Any antibody binding assay known in the art can be used to measure the percentage of trimer formation of a recombinant HIV-1 Env protein of the invention, such as ELISA, AlphaLISA, etc.

The amount of HIV-1 Env trimeric complexes formed and the total amount of envelope protein expressed can also be determined using, for example, chromatographic techniques (e.g., size exclusion chromatography multi-angle light scattering (SEC-MALS) that can separate the trimeric complex form from any other forms of the HIV-1 Env protein, e.g., the monomer form.

An HIV-1 Env trimeric complex can comprise three gp120-gp41 protomers comprising a gp120 polypeptide and a gp41 extracellular domain. Mature gp120 includes HIV-1 Env residues from about 31-511 (wherein the amino acids are numbered by HXB2 numbering), and contains most of the extemal, surface exposed, domains of the HIV-1 Env trimeric complex. The gp120 portion of the trimeric complex can bind to cellular CD4 receptors and can bind to cellular chemokine receptors. A mature gp120 polypeptide is an extracellular polypeptide that interacts with the gp41 extracellular domain (approximately HIV-1 Env positions 512-644) to form an HIV-1 Env protomer that trimerizes to form an HIV-1 Env trimeric complex. Mature gp41 comprises approximately HIV-1 Env amino acids 512-856, and includes cytosolic, transmembrane, and extracellular-domains. The gp41 extracellular domain comprises HIV-1 Env residues from about 512-644.

In an embodiment an HIV-1 Env protein comprises one or more of the mutations disclosed herein in an extracellular domain. In an embodiment an HIV1-Env protein comprises a fragment of an HIV-1 Env protein, which includes at least one extracellular domain or a portion of an extracellular domain having one or more of the mutations described herein. An extracellular fragment can be about 20, 50, 75, 100, 125, 150, 175, 200, 225, 250, 275, 300, 325, 350, 375, 400, 425, 450, or more amino acids in length.

In an embodiment, the N-terminal residue of the gp120 polypeptide is one of HIV-1 Env positions 1-35 (i.e., one or more of amino acids 1-34 can be absent). In an embodiment, the C-terminal residue of the gp120 polypeptide is one of HIV-1 Env positions 503-512 (i.e., one or more of amino acids 504-512 can be truncated or removed). In an embodiment, the N-terminal residue of the gp41 extracellular domain is one of HIV-1 Env positions 512-522 (i.e., one or more of amino acids 512-521 are absent). In an embodiment the C-terminal residue of the gp41 extracellular domain is one of HIV-1 Env positions 640-683 (i.e., one or more of amino acids 640-683 is removed). All numbering refers to HXB2 numbering.

Truncations of the C-terminus of HIV-1 Env can be useful to (i) increase expression and/or (ii) make a soluble extracellular fragment.

An HIV-1 Env trimeric complex can be stable in a mature closed conformation. An HIV-1 Env trimeric complex comprises at least one mutant HIV-1 Env protein or fragment described herein and can exhibit increased retention of the mature closed conformation upon CD4 binding compared to a corresponding wild-type or naturally occurring HIV-1 Env trimeric complex.

A HIV-1 Env trimeric complex stabilized in the mature closed conformation can have at least about 60, 70, 80, 90, 95, 98, 99% or more reduced transition to the CD4-bound open conformation upon CD4 binding compared to a corresponding native HIV-1 Env trimeric complex. The stabilization of the mature closed conformation by one or more mutations described herein can be, for example, energetic stabilization (for example, reducing the energy of the mature closed conformation relative to the CD4-bound open conformation) and/or kinetic stabilization (for example, reducing the rate of transition from the mature closed conformation to the open conformation) and/or reduced conformational heterogeneity (for example, a greater fraction of the expressed protein is in the closed conformation). Additionally, stabilization of the HIV-1 Env trimeric complex in the mature closed conformation can include an increase in resistance to denaturation compared to a corresponding native HIV-1 Env trimeric complex.

In an embodiment, the inclusion of one or more mutations described herein increases the pool of HIV-1 Env trimeric complexes present in the closed state as compared to wild-type or naturally occurring HIV-1 Env trimeric complexes. That is, use of HIV-1 Env proteins or fragments thereof having one or more mutations described herein can result in about 5, 10, 20, 30, 40, 50, 60, 70, 80, 90% or more HIV-1 Env trimeric complexes present in the closed state as compared to the use of wild-type or naturally occurring HIV-1 proteins or fragments thereof.

Methods of determining if a HIV-1 Env trimeric complex is in the mature closed conformation include, for example, negative stain electron microscopy and antibody binding assays using a mature closed conformation specific antibody, such as VRC26 or PGT145. Methods of determining if a HIV-1 Env trimeric complex is in the CD4-bound open conformation include for example, negative stain electron microscopy and antibody binding assays using a CD4-bound open conformation specific antibody, such as 17b, which binds to a CD4-induced epitope.

In an embodiment an HIV-1 Env trimeric complex can comprise a non-natural disulfide bond between cysteine substitutions at positions 201 and 433, a non-natural disulfide bond between cysteine substitutions at positions 501 and 605, and a proline substitution at position 559.

In an embodiment an HIV-1 Env trimeric complex does not specifically bind to a CD4-induced antibody when incubated with a molar excess of soluble CD4.

### Immunogen

An embodiment provides an immunogen comprising one or more of the HIV-1 Env proteins, fragments thereof, trimeric complexes or portions thereof described herein. An immunogen can also be a vector, nucleic acid molecule, or host cell as described herein. An immunogen can induce an immune response in a mammal, including for example, humans infected with HIV-1 or at risk of HIV-1 infection. Administration of an immunogen can lead to protective immunity and/or proactive immunity against HIV-1.

An immune response is a response of a cell of the immune system, such as a B cell, T cell, or monocyte, to a stimulus. In one embodiment, the response is specific for a particular HIV-1 Env antigen (an “antigen-specific response”). In one embodiment, an immune response is a T cell response, such as a CD4+ response or a CD8+ response. In another embodiment, the response is a B cell response, and results in the production of specific antibodies. In an embodiment, an HIV-1 Env protein, fragment thereof, HIV-1 Env trimeric complex or portion thereof as described herein comprises an epitope or other construct such that the protein or fragment thereof will bind an MHC molecule and induce an immune response, such as a cytotoxic T lymphocyte (“CTL”) response, and/or a B cell response (for example, antibody production), and/or a T-helper lymphocyte response against the antigen from which the protein or fragment thereof is derived.

### Nucleic Acid Molecules, Vectors, and Host Cells

Embodiments include nucleic acid molecules encoding the mutant HIV-1 Env protein, fragments thereof, HIV-1 Env trimeric complex or portion thereof disclosed herein. Polynucleotides contain less than an entire viral genome and can be single- or double-stranded nucleic acids. A polynucleotide can be RNA, DNA, cDNA, genomic DNA, chemically synthesized RNA or DNA or combinations thereof. A polynucleotide can comprise, for example, a gene, open reading frame, non-coding region, or regulatory element.

A gene is any polynudeotide molecule that encodes a protein or fragments thereof, optionally including one or more regulatory elements preceding (5′ non-coding sequences) and following (3′ non-coding sequences) the coding sequence. In one embodiment, a gene does not include regulatory elements preceding and following the coding sequence. A naturally occurring or wild-type gene refers to a gene as found in nature, optionally with its own regulatory elements preceding and following the coding sequence. A chimeric or recombinant gene refers to any gene that is not a naturally occurring or wild-type gene, optionally comprising regulatory elements preceding and following the coding sequence, wherein the coding sequences and/or the regulatory elements, in whole or in part, are not found together in nature. Thus, a chimeric gene or recombinant gene comprise regulatory elements and coding sequences that are derived from different sources or regulatory elements and coding sequences that are derived from the same source, but arranged differently than is found in nature. A gene can encompass full-length gene sequences (e.g., as found in nature and/or a gene sequence encoding a full-length polypeptide or protein) and can also encompass partial gene sequences (e.g., a fragment of the gene sequence found in nature and/or a gene sequence encoding a protein or fragment of a polypeptide or protein). A gene can include modified gene sequences (e.g., modified as compared to the sequence found in nature). Thus, a gene is not limited to the natural or full-length gene sequence found in nature.

Polynucleotides can be purified free of other components, such as proteins, lipids and other polynucleotides. For example, the polynucleotide can be 50%, 75%, 90%, 95%, 96%, 97%, 98%, 99%, or 100% purified. A polynucleotide existing among hundreds to millions of other polynucleotide molecules within, for example, cDNA or genomic libraries, or gel slices containing a genomic DNA restriction digest are not to be considered a purified polynucleotide. Polynucleotides can encode the proteins described herein (e.g., mutant HIV-1 Env proteins and fragments thereof).

Polynucleotides can comprise additional heterologous nucleotides that do not naturally occur contiguously with the polynucleotides. As used herein the term “heterologous” refers to a combination of elements that are not naturally occurring or that are obtained from different sources.

Polynucleotides can comprise other nucleotide sequences, such as sequences coding for linkers, signal sequences, TMR stop transfer sequences, transmembrane domains, or ligands useful in protein purification such as glutathione-S-transferase, histidine tag, and Staphylococcal protein A.

Polynucleotides can be isolated. An isolated polynucleotide is a naturally-occurring polynucleotide that is not immediately contiguous with one or both of the 5′ and 3′ flanking genomic sequences that it is naturally associated with. An isolated polynucleotide can be, for example, a recombinant DNA molecule of any length, provided that the nucleic acid sequences naturally found immediately flanking the recombinant DNA molecule in a naturally-occurring genome is removed or absent. Isolated polynucleotides also include non-naturally occurring nucleic acid molecules. Polynucleotides can encode full-length proteins, polypeptide fragments, and variant or fusion polypeptides.

Degenerate polynucleotide sequences encoding polypeptides described herein, as well as homologous nucleotide sequences that are at least about 80, or about 85, 90, 91, 92, 93, 94, 95, 96, 97, 98, or 99% identical to polynucleotides described herein and the complements thereof are also polynucleotides. Degenerate nucleotide sequences are polynucleotides that encode a protein described herein or fragments thereof but differ in nucleic acid sequence from the wild-type polynucleotide sequence, due to the degeneracy of the genetic code. Complementary DNA (cDNA) molecules, species homologs, and variants of polynucleotides that encode biologically functional polypeptides also are polynucleotides.

Polynucleotides can be obtained from nucleic acid sequences present in, for example, an HIV virion. Polynucleotides can also be synthesized in the laboratory, for example, using an automatic synthesizer. An amplification method such as PCR can be used to amplify polynucleotides from either genomic DNA or cDNA encoding the polypeptides.

Polynucleotides can comprise coding sequences for naturally occurring polypeptides or can encode altered sequences that do not occur in nature. Unless otherwise indicated, the term polynucleotide or gene includes reference to the specified sequence as well as the complementary sequence thereof.

An embodiment includes a vector comprising an HIV-1 Env polynucleotide that encodes a mutant HIV-1 Env protein, fragment thereof, HIV-1 Env trimeric complex or portion thereof as disclosed herein. A vector is used to introduce a nucleic acid molecule into a host cell, thereby producing a transformed host cell. Recombinant vectors are vectors having recombinant DNA. A vector can include nucleic acid sequences that permit it to replicate in a host cell, such as an origin of replication. A vector can also include one or more selectable marker genes and other genetic elements known in the art.

If desired, polynucleotides can be cloned into an expression vector comprising expression control elements, including for example, origins of replication, promoters, enhancers, or other regulatory elements that drive expression of the polynucleotides in host cells. A vector can be, for example, a virus (e.g., adenovirus or poxvirus), naked DNA, oligonucleotide, cationic lipid (e.g., liposome), cationic polymer (e.g., polysome), virosome, nanoparticle, or dentrimer. Other viral vectors include adeno-associated virus vectors, retrovirus vectors, poxviruses, vaccinia virus, herpesviruses, togaviruses, picomaviruses, and baculoviruses. Other vectors include bacteriophages, phagemids, yeast artificial chromosomes, bacterial artificial chromosomes, virus-like particles, cosmids (plasmids into which phage lambda cos sites have been inserted) and replicons (genetic elements that are capable of replication under their own control in a cell).

The nucleic acid molecules of a vector can be encapsulated in a lipid membrane or by structural proteins (e.g., capsid proteins), that can include one or more viral polypeptides (e.g., an HIV-1 Env protein or portion thereof). A vector can be used to infect cells of a subject, such that translation of the heterologous gene(s) of the vector occurs. In an embodiment an HIV-1 Env trimeric complex is formed.

Naked DNA or oligonudeotides encoding one or more of the HIV-1 Env proteins, fragments thereof, HIV-1 trimeric complexes or portions thereof described herein can also be used to express HIV-1 Env proteins in a cell or a subject to promote formation of HIV-1 Env trimeric complexes. See, e.g., Cohen, Science 259:1691-1692 (1993); Fynan et al., Proc. Natl. Acad. Sci. USA, 90:11478 (1993); and Wolff et al., BioTechniques 11:474485 (1991).

A virus-like particle (VLP) can comprise one or more HIV-1 Env proteins, fragments thereof, or HIV-1 Env trimeric complexes as described herein. VLPs lack the viral components that are required for virus replication and thus represent a highly attenuated form of a virus. The VLP can display an HIV-1 Env protein, fragment thereof, trimeric complex or portion thereof that is capable of eliciting an immune response to HIV-1 Env when administered to a subject. Virus like particles and methods of their production are known to the person of ordinary skill in the art, and viral proteins from several viruses are known to form VLPs, including human papillomavirus, HIV, Semliki-Forest virus, human polyomavirus, rotavirus, and others. The virus like particle can include any of the recombinant HIV-1 Env trimeric complexes or immunogenic fragments thereof that are disclosed herein.

An embodiment provides a host cell comprising one or more vectors or nucleic acid molecules as described above. A recombinant host cell, transgenic host cell, or transformed host cell is a cell into which one or more foreign or exogenous nucleic acid molecules, synthetic nucleic acid molecules, or plasmids have been introduced or inserted into the cell. The one or more foreign nucleic acid molecules, synthetic nucleic acid molecules, or plasmids do not occur in the host cell in nature. The cell may be prokaryotic or eukaryotic. The term also includes any progeny of the host cell. It is understood that all progeny may not be identical to the parental cell since there may be mutations that occur during replication. However, such progeny are included when the term “host cell” is used.

### Methods of Producing HIV-1 Env Proteins

An embodiment provides methods of producing HIV-1 Env proteins, fragments thereof, HIV-1 trimeric complexes or portions thereof comprising one or more of the mutations described herein. In an embodiment nucleic acid molecules capable of expressing HIV-1 Env proteins, fragments thereof, HIV-1 Env trimeric complexes or portions thereof are cloned into one or more vectors. In an embodiment nucleic acid molecules encoding HIV-1 Env gp120 subunits are cloned into a first vector and nucleic acid molecules encoding HIV-1 Env gp140 are cloned into a second vector. Both vectors can be introduced into host cells. In an embodiment the nucleic acid molecules encoding HIV-1 proteins or fragments thereof are codon optimized for expression in human cells.

One or more host cells can be cultured in an appropriate medium to produce HIV-1 Env proteins, fragments thereof, HIV-1 trimeric complexes or portions thereof. A host cell can be a mammalian cell having the ability to glycosylate proteins.

An embodiment provides a method of producing a stable HIV-1 Env trimer in a closed conformation. The method comprises making one or more of the amino acid mutations or sets of mutations disclosed herein in an HIV-1 Env protein, fragment thereof, HIV-1 Env trimeric complex or portion thereof and expressing the protein in a host cell.

### Screening Methods

An embodiment provides methods of screening a compound or test agent for binding to one or more HIV-1 Env proteins, fragments thereof, HIV-1 Env complexes or portions thereof comprising one or more of the mutations described herein. The one or more mutant HIV-1 Env proteins, fragments thereof, trimeric complexes or portions thereof are contacted with one or more test agents or compounds. The ability of the test agent or compound to bind to the one or more mutant HIV-1 Env proteins, fragments, trimeric complexes or portions thereof is determined. The test agent can be any type of compound, molecule, biological molecule, or drug.

Binding assays such as competitive binding assays and direct binding assays are well known to those of skill in the art.

A plurality of mutant HIV-1 Env proteins (e.g., 2, 5, 10, 15, or more mutant HIV-1 Env proteins, fragments thereof, trimeric complexes or portions thereof disclosed herein) can be screened.

A compound or test agent can be tested for inhibition of an HIV-1-mediated activity. An HIV-1-mediated activity can be, for example, viral spread, infection, or cell fusion. Cell fusion may be, for example, target cell entry or syncytial formation. In an embodiment, the compound or test agent inhibits an HIV-mediated activity. The compound or test agent can be provided in a library or display library.

In an embodiment, a screening step can also serve as the step of recovering a test agent or compound that binds to the mutant HIV-1 Env protein, fragment thereof, or trimeric compound.

Methods well known to those skilled in the art can be applied to screening of libraries or display libraries of test agents or compounds. Examples include solid-phase screening methods and liquid-phase screening methods. Solid-phase screening methods can involve, for example, immobilizing test agents or compounds onto a solid phase, and contacting mutant HIV-1 Env proteins, fragments, trimeric complexes or portions thereof contained in a liquid phase with the test agents or compounds and removing unbound mutant HIV-1 Env proteins, fragments, trimeric complexes or portions thereof and nonspecifically bound mutant HIV-1 Env proteins, fragments thereof, trimeric complexes or portions thereof and then selectively separating mutant HIV-1 Env proteins, fragments, trimeric complexes or portions thereof bound with the test agent or compound to screen for a protein, fragment, trimeric complex or portion thereof having, for example, a desired binding activity. A liquid-phase screening method can involve, for example, contacting mutant HIV-1 Env proteins, fragments thereof, trimeric complexes or portions thereof with test agents in a solution, removing unbound mutant HIV-1 Env proteins, fragments thereof, trimeric complexes and portions thereof and nonspecifically bound HIV-1 Env proteins, fragments thereof, trimeric complexes and portions thereof and then selectively separating the HIV-1 Env proteins, fragments thereof, trimeric complexes or portions thereof bound with test agents or compounds.

### Nanodiscs

In an embodiment, an HIV-1 Env protein, fragment thereof, HIV-1 trimeric complex, portion thereof, or combinations thereof can be presented as a membrane protein in nanodiscs. See, e.g., Bayburt et al., J. Struct. Biol. (1998); 123:37; Civjan et al., BioTechniques (2003) 35:556; Hagn et al., J. Am. Chem. Soc. (2013) 135:1919.

Nanodiscs have a phospholipid bilayer system held together by membrane scaffold proteins (MSPs), which wrap around a patch of a lipid bilayer to form a disc-like particle or nanodisc. MSPs have a hydrophobic surface facing the lipids, and a hydrophilic surface facing outward. Nanodiscs are therefore highly soluble in aqueous solutions. Once assembled into nanodiscs, membrane proteins can be kept in solution in the absence of detergents.

MSPs can be, for example, truncated forms of apolipoprotein (apo) A-I, MSP1D1, MSP1E3D1, MSP2N2, MSP2N3, or MSP1D1dH5. Nanodiscs can be about 7-17 nm in diameter depending on the type of MSP used. MSPs can be derived from for example, mouse, rat or human apo A-1 proteins. Use of mouse or rat apo A-1 proteins can improve antibody specificity when human HIV-1 Env target protein-nanodisc complexes are used for immunization.

Nanodiscs can be used to reconstitute HIV-1 Env in an artificial environment resembling the native membrane. These nanodisc-stabilized proteins can be directly purified by standard chromatographic procedures. The resulting purified membrane protein:nanodisc complex can be used in screening applications that require access to both the physiologically intracellular and extracellular surfaces of the protein and thus allows unrestricted access of antagonists, agonists, and other interaction partners. The nanodiscs can also be used as an HIV-1 Env immunogen or HIV-1 Env vaccine.

Nanodiscs can be made using cell-free expression systems. An HIV-1 Env protein, fragment thereof, HIV-1 Env trimeric complex, portions thereof or combinations thereof can be expressed from, for example, a plasmid. Pre-assembled nanodiscs are supplied in the mixture that integrate the nascent HIV-1 Env protein. Nanodiscs can also be made using a two-step reconstitution of detergent-solubilized proteins. Purified HIV-1 Env proteins, fragments thereof, HIV-1 Env trimeric complexes, portions thereof or combinations thereof are combined with a suitable detergent, and membrane scaffold proteins and phospholipids are added. Nanodiscs containing the membrane protein form spontaneously, and can be purified by affinity or size exclusion chromatography. Nanodiscs can also be made via direct solubilization from membranes expressing HIV-1 Env proteins, fragments thereof. HIV-1 Env trimeric complexes, portions thereof, or combinations thereof. Starting from membranes expressing the HIV-1 Env protein, detergent and membrane scaffold protein are added. Membrane phospholipids, HIV-1 Env protein and MSP assemble to form the nanodisc complex.

Phospholipids such as dimyristoyl-glycero-phosphocholine (DMPC), palmitoyl-oleoyl-phosphatidylcholine (POPC), and many other phospholipids can be used when making nanodiscs.

An embodiment provides a nanodisc comprising one or more of HIV-1 Env proteins, fragments thereof, HIV-1 Env trimeric complexes, portions thereof, or combinations thereof as described herein, a membrane scaffold protein; and one or more phospholipids. In an embodiment, the nanodiscs can be used to screen test agents or compounds for binding or biological activity. In an embodiment, the nanodiscs can be used as an immunogen or vaccine.

### Pharmaceutical Compositions

A pharmaceutical composition can comprise a mutant HIV-1 Env protein or fragment thereof (which comprise at least one mutation described herein), an HIV-1 Env trimeric complex comprising at least on mutant HIV-1 Env protein or portion thereof (which comprises at least one mutation described herein), nucleic acid molecule as described herein, a vector as described herein, a host cell as described herein, or a combination thereof combined with a pharmaceutically acceptable carrier.

Pharmaceutically acceptable carriers are well known in the art. See e.g., Remington's Pharmaceutical Sciences, by E. W. Martin, Mack Publishing Co., Easton, Pa., 19th Edition, 1995, which describes compositions and formulations suitable for pharmaceutical delivery.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. In particular embodiments, a carrier may be sterile, and/or suspended or otherwise contained in a unit dosage form containing one or more measured doses of the composition suitable to induce the desired anti-HIV-1 immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

A pharmaceutical composition can comprise an adjuvant to enhance antigenicity. An adjuvant can comprise, for example, a suspension of minerals (alum, aluminum hydroxide, or phosphate); or water-in-oil emulsion, for example, in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). Immunostimulatory oligonucleotides (such as those including a CpG motif) can also be used as adjuvants. Adjuvants can include biological molecules, such as costimulatory molecules. Exemplary adjuvants include IL-2, RANTES, GM-CSF, TNF-α, IFN-γ, G-CSF, LFA-3, CD72, B7-1, B7-2, OX-40L, SA-4-1 BBL and toll-like receptor (TLR) agonists, such as TLR-9 agonists. Adjuvants are well known in the art. See, e.g., Singh (ed.) Vaccine Adjuvants and Delivery Systems. Wiley-Interscience, 2007).

A pharmaceutical composition can comprise a detergent, such as a non-ionic detergent (e.g., a polyethylene type detergent).

### Methods of Eliciting Immune Responses, Prevention, and Treatment

An embodiment provides a method for eliciting an immune response against an HIV-1 infected cell in a subject (e.g., a mammal such as a human) comprising administering to the subject a therapeutically effective amount of an HIV-1 Env protein, fragment thereof, HIV-1 Env trimeric complex, or portion thereof comprising one or more mutations described herein such that an immune response is elicited in the subject. In an embodiment a pharmaceutical composition as described herein can be used to elicit an immune response against an HIV-1 infected cell in a subject.

An embodiment provides a method for delaying the onset of, or slowing the rate of progression of, an HIV-1-related disease or symptom in an HIV-1-infected subject. Examples of symptoms of diseases (e.g., AIDS) include, for example, fever, muscle aches, coughing, sneezing, runny nose, sore throat, headache, chills, diarrhea, vomiting, rash, weakness, dizziness, bleeding under the skin, in internal organs, or from body orifices like the mouth, eyes, or ears, shock, nervous system malfunction, delirium, seizures, renal failure, personality changes, neck stiffness, dehydration, seizures, lethargy, paralysis of the limbs, confusion, back pain, loss of sensation, impaired bladder and bowel function, and sleepiness that can progress into coma or death.

The method comprises administering to the subject (e.g., a mammal such as a human) a therapeutically effective amount of an HIV-1 Env protein, fragment thereof, HIV-1 Env trimeric complex or portion thereof comprising one or more mutations described herein such that the onset of an HIV-1 disease or symptom is delayed or the progression of HIV-1 symptoms are slowed in comparison to an HIV-1 positive subject who does not receive the administration. In an embodiment a pharmaceutical composition as described herein can be used in the methods.

A therapeutically effective amount refers to an amount of a composition described herein that, when administered to a subject for treating a disease or disorder or at least one symptom of the disease or disorder, is sufficient to affect such disease, disorder, or symptom. A therapeutically effective amount can vary depending, for example, on the composition that is administered, the disease, disorder, and/or symptoms of the disease or disorder, severity of the disease, disorder, and/or symptoms of the disease or disorder, the age, weight, and/or health of the subject to be treated, the capacity of the individual's immune system to synthesize antibodies, the degree of protection desired, the formulation of the composition, the treating doctor's assessment of the medical situation, and other relevant factors. An appropriate amount in any given instance can be readily ascertained by those skilled in the art or capable of determination by routine experimentation. It is expected that the amount will fall in a relatively broad range that can be determined through routine trials. For example, in the context of administering a therapeutic agent (e.g., HIV-Env protein, fragments thereof, HIV-1 Env trimeric complexes, portions thereof, and pharmaceutical compositions described herein), the therapeutically effective amount is an amount sufficient to achieve a reduction in the level of HIV (e.g., as measured by a stabilization or decrease in HIV titer compared to a non-treated control), and/or an increase in the level of neutralizing anti-HIV antisera (e.g., as measured by an increase in serum neutralizing antibody levels relative to a non-treated control in a luciferase-based virus neutralization assay) as compared to a response obtained without administration of a therapeutic agent described herein, and/or to prevent the propagation of a HIV-1 in a subject (e.g., a human) having an increased risk of viral infection. I

In an embodiment, a therapeutically effective amount provides a therapeutic effect without causing a substantial cytotoxic effect in the subject. In general, a therapeutically effective amount of a composition administered to a subject (e.g., a human) will vary depending upon a number of factors associated with that subject, for example the overall health of the subject, the condition to be treated, or the severity of the condition. A therapeutically effective amount of a composition can be determined by varying the dosage of the product and measuring the resulting therapeutic response.

Administering means giving a dosage of a pharmaceutical composition as described herein to a subject. Administration can be done, for example, intramuscularly, intravenously, intradermally, percutaneously, intraarterially, intraperitoneally, intralesionally, intracranially, intraarticulariy, intraprostatically, intrapleurally, intratracheally, intranasally, intravitreally, intravaginally, intrarectally, topically, intratumorally, peritoneally, subcutaneously, subconjunctivally, intravesicularily, mucosally, intrapericardially, intraumbilically, intraocularly, orally, topically, locally, by inhalation, by injection, by infusion, by continuous infusion, by localized perfusion bathing target cells directly, by catheter, by lavage, by gavage, in creams, or in lipid compositions.

In an embodiment an HIV-1 Env protein, fragment thereof, HIV-1 Env trimeric complex, portion thereof, or pharmaceutical composition induces a neutralizing immune response or a broadly neutralizing immune response to HIV-1 Env in the subject

In an embodiment an administration of the compositions described herein to a subject causes a reduction or decrease of an HIV-mediated activity (e.g., infection, fusion (e.g., target cell entry and/or syncytia formation), viral spread, etc.) and/or a decrease in viral titer). HIV-mediated activity and/or HIV titer may be decreased by about 5%, 10%, 20%, 30%, 40%, 50%, 60%, 70%, 80%, 90%, 95%, 98%, 99% or more compared to that of a control subject (e.g., an untreated subject or a subject treated with a placebo).

An embodiment provides a vaccine, which is an HIV-1 Env protein, fragment thereof, HIV-1 Env trimeric complex, portion thereof, or pharmaceutical composition as described herein that can provoke an immune response. Administration of a vaccine to a subject can confer at least some protective immunity against HIV-1 infection (e.g., enhancement of resistance to new infection, complete resistance to new infection, or reduction or elimination of clinical severity of the disease or symptoms).

An embodiment provides a method for preventing a subject (e.g., a mammal such as a human) from becoming infected with HIV-1. The method comprises administering to the subject a prophylactically effective amount of an HIV-1 Env protein, fragment thereof, HIV-1 Env trimeric complex, or portion thereof comprising one or more mutations described herein such that a protective immune response is elicited in the subject. In an embodiment a pharmaceutical composition as described herein can be used to elicit a protective immune response. The subject can be prevented from becoming infected with at least 1, 2, 3, 4, 5, 6, or more strains of HIV-1.

A prophylactically effective amount is any amount of an agent (e.g., an HIV-1 Env protein, fragment thereof, HIV-1 Env trimeric complex, portion thereof or pharmaceutical composition described herein) which, when administered to a subject prone to suffer from a disease or disorder, inhibits or prevents the onset of the disease or disorder. The prophylactically effective amount will vary with the subject being treated, the condition to be treated, the agent delivered, and the route of delivery. A person of ordinary skill in the art can perform routine titration experiments to determine such an amount. A prophylactic treatment is a treatment administered to a subject who does not exhibit signs of a disease or exhibits only early signs for the purpose of decreasing the risk of developing pathology.

A protective immunological response or protective immunity can be demonstrated by either a reduction or lack of clinical signs or symptoms normally displayed by an infected host, a quicker recovery time, and/or a lowered duration of infectivity or lowered pathogen titer in the tissues or body fluids or excretions of the infected host.

An embodiment provides a method for reducing the likelihood of a subject becoming infected with HIV-1. The method comprises administering to the subject an amount effective to reduce the likelihood of the subject becoming infected with HIV-1 of an HIV-1 Env protein, fragment thereof, HIV-1 Env trimeric complex or portion thereof comprising one or more mutations described herein such that an immune response is elicited in the subject. In an embodiment a pharmaceutical composition as described herein can be used to elicit an immune response. In an embodiment, the subject has been exposed to HIV-1.

An embodiment provides a DNA vaccine comprising one or more of the nucleic acid molecules encoding an HIV-1 Env protein, fragment thereof, HIV-1 Env trimeric complex or portion thereof, wherein then protein, fragment, complex or portion comprises one or more of the mutations described herein together with a pharmaceutically acceptable adjuvant. A DNA vaccine comprises genetically engineered DNA that is delivered directly to cells to produce an HIV-1 Env antigen (e.g., an HIV-1 Env protein, fragment thereof, HIV-1 Env trimeric complex or portion thereof as described herein), such that an immune response is generated. In an embodiment, the immune response is a protective immune response.

An embodiment provides a recombinant viral vector vaccine, which comprises a recombinant viral vector comprising one or more of the nucleic acid molecules encoding an HIV-1 Env protein, fragment thereof, HIV-1 Env trimeric complex or portion thereof, wherein then protein, fragment, complex or portion comprises one or more of the mutations described herein and a pharmaceutical acceptable adjuvant. The recombinant viral vector can be, for example, vaccinia vector, adenovirus vector, adena-associated virus vector, sendai virus vector, herpes simplex virus vector, human papillomavirus vector, retroviral vector or other viral vector. A recombinant viral vector can be a replicative viral vector.

An embodiment provides a recombinant bacterial vector vaccine, which comprises a recombinant bacterial vector comprising one or more of the nucleic acid molecules encoding an HIV-1 Env protein, fragment thereof, HIV-1 Env trimeric complex or portion thereof, wherein then protein, fragment, complex or portion comprises one or more of the mutations described herein and a pharmaceutical acceptable adjuvant.

Ex vivo transfection or transduction of cells can also be used to deliver the HIV-1 Env proteins, fragments thereof, HIV-1 Env trimeric complexes or portions thereof to a subject. The cells can be delivered into a subject to allow for the expression of one or more of the HIV-1 Env proteins, fragments thereof, trimeric complexes or portions thereof described herein. In embodiments, cells can be autologous or heterologous to the treated subject. Cells can be transfected or transduced ex vivo with, for example, one or more vectors or nucleic acid molecules described herein to allow for the temporal or permanent expression of one or more of the HIV-1 Env proteins, fragments thereof, HIV-1 Env trimeric complexes, or portions thereof in the treated subject. Once the modified cells are administered to the subject, the one or more vectors or nucleic acid molecules will be expressed, eliciting protective or therapeutic immune responses directed against the HIV-1 Env proteins or HIV-1 Env trimeric complexes.

Cells that can be isolated and transfected or transduced ex vivo can be, for example, blood cells, skin cells, fibroblasts, endothelial cells, skeletal muscle cells, hepatocytes, prostate epithelial cells, vascular endothelial cells, and totipotent, pluripotent, multipotent, or unipotent stem cells.

In an embodiment the HIV-1 Env protein, fragment thereof, HIV-1 Env trimeric complex, portion thereof, or pharmaceutical composition can be administered as part of a prime-boost regimen. In an embodiment, the immune response triggered by a single administration (prime) of a composition described herein may not be sufficiently potent and/or persistent to provide effective protection. Therefore, repeated administration (boost), such that a prime-boost regimen is established, may significantly enhance humoral and cellular responses to the antigen(s) of the composition.

The booster is administered to the subject after the primer. A skilled artisan will understand a suitable time interval between administration of the primer composition and the booster composition. In some embodiments, the primer composition, the booster composition, or both primer composition and the booster composition additionally include an adjuvant. In one embodiment, the primer composition is a DNA-based vaccine (or other vaccine based on gene delivery), and the booster composition is a protein-based vaccine.

### Dosages

HIV-1 Env proteins, fragments thereof, HIV-1 Env trimeric complexes, portions thereof, or pharmaceutical compositions can be administered in a prophylactically effective amount or a therapeutically effective amount that provides an immunogenic response and/or protective effect against HIV-1. A protein composition can be administered at between about 1 μg and about 1 mg of protein, or between about 50 μg and about 300 μg of protein.

A viral vector capable of expressing HIV-1 Env proteins, fragments thereof, HIV-1 Env trimeric complexes or portions thereof can be administered at least about 1×10³ viral particles (vp)/dose, between about 1×10¹ and about 1×10¹⁴ vp/dose, between about 1×10³ and about 1×10¹² vp/dose, or between about 1×10⁵ and about 1×10¹¹ vp/dose.

Levels of induced immunity or immune response provided by the compositions can be monitored by, for example, measuring amounts of HIV-1 neutralizing anti-HIV antibodies. The dosages may then be adjusted or repeated as necessary to trigger the desired level of immune response.

Where a subject is administered an HIV-1 protein, fragment thereof, HIV-1 Env trimeric complex, portion thereof, or pharmaceutical composition the efficacy of treatment can be determined by monitoring the level of the HIV-1 Env proteins, fragments thereof, HIV-1 Env trimeric complexes or portions thereof expressed by or present in a subject following administration. For example, the blood or lymph of a subject can be tested for the HIV-1 Env proteins, using for example, standard assays known in the art (see, e.g., Human Interferon-Alpha Multi-Species ELISA kit and Human Interferon-Alpha Serum Sample kit from Pestka Biomedical Laboratories (PBL), Piscataway, N.J.).

A single dose of one or more of the pharmaceutical compositions described herein can achieve therapy in subjects. Multiple doses (e.g., 2, 3, 4, 5, or more doses) can also be administered to subjects.

HIV-1 proteins, fragments thereof, HIV-1 Env trimeric complexes, portions thereof or pharmaceutical compositions can be administered, for example, every 1, 2, 3, 4, 5, 6, 7, 8, 9, 10, 15, 20, 30, 40, 45, 50, 55, or 60 minutes, 2, 4, 6, 10, 15, or 24 hours, 2, 3, 5, or 7 days, 2, 4, 6 or 8 weeks, or even 3, 4, or 6 months pre-exposure or pre-diagnosis, or may be administered to the subject every 15-30 minutes or 1, 2, 3, 4, 5, 6, 7, 8, 9, 10, 12, 20, 24, 48, or 72 hours, 2, 3, 5, or 7 days, 2, 4, 6 or 8 weeks, 3, 4, 6, or 9 months, 1, 2, 3, 4, 5, 6, 7, 8, 9, 10, 15, 20 years or longer post-diagnosis or post-exposure or to HIV-1. A subject can be administered one or more doses of the HIV-1 proteins, fragments thereof, HIV-1 Env trimeric complexes, portions thereof or pharmaceutical compositions once daily, weekly, monthly, or yearly. When treating an HIV-1 infection, the compositions can be administered to the subject either before the occurrence of symptoms of an HIV infection or disease/syndrome (e.g., acquired immune deficiency syndrome (AIDS)) or a definitive diagnosis, or after diagnosis or symptoms become evident. HIV-1 proteins, fragments thereof, HIV-1 Env trimeric complexes, portions thereof or pharmaceutical compositions can be administered, for example, immediately after diagnosis or the clinical recognition of symptoms or every 2, 4, 6, 10, 15, or 24 hours, 2, 3, 5, or 7 days, 2, 4, 6 or 8 weeks, or even 3, 4, or 6 months after diagnosis or detection of symptoms.

### Antibodies

An embodiment provides antibodies (i.e., an immunoglobulin or an antigen-binding fragment) that specifically bind and recognize an HIV-1 Env protein, an antigenic fragment thereof, or an HIV-1 Env trimeric complex or antigenic fragment thereof. The HIV-1 Env protein, antigenic fragment thereof or HIV-1 Env trimeric complex or antigenic fragment thereof comprise one or more of the mutations described herein. In an embodiment, antibodies are provided that specifically bind to an HIV-1 Env trimeric complex in a closed conformation, wherein the HIV-1 Env trimeric complex comprises one or more of the mutations described herein.

Antibodies include monoclonal antibodies, polyclonal antibodies, multispecific antibodies (e.g., bispecific antibodies), and antibody fragments, as long as they have antigen-binding activity. In an embodiment, antibodies and fragments thereof can be chimeric antibodies (see, e.g., U.S. Pat. No. 5,482,856), humanized antibodies (see, e.g., Jones et al., Nature 321:522 (1986); Reichmann et al., Nature 332:323 (1988)); Presta, Curr. Op. Struct. Biol. 2:593 (1992)), or human antibodies. Human antibodies can be made by, for example, direct immortalization, phage display, transgenic mice, or a Trimera methodology, see e.g., Reisener et al., Trends Biotechnol. 16:242-246 (1998).

Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies. See, e.g., Kontermann & Dubel (Ed), Antibody Engineering, Vols. 1-2, 2^{nd} Ed., Springer Press, 2010).

In an embodiment, an antibody or specific binding fragment thereof that specifically binds HIV-1 Env protein, fragment thereof, HIV-1 Env trimeric complex, or portion thereof having one or more mutations described herein has a broader and higher neutralization activity to HIV-1 virus when compared to an antibody or fragment produced by induction with a wild-type (i.e., naturally occurring) HIV-1 Env protein, fragment thereof, HIV-1 Env trimeric complex, or portion thereof.

An embodiment provides methods of identifying antibodies that specifically bind to HIV-1 Env proteins or HIV-1 Env trimeric complexes described herein. An effective amount of an immunogen is administered to B cells in an in vitro cell culture system to generate antibodies that neutralize HIV-1 virus. An effective amount is an amount that is sufficient to generate antibodies specific for HIV-1 Env protein, fragment thereof, HIV-1 trimeric complex, or fragment thereof. In an embodiment the HIV-1 virus is heterologous to the virus strain or subtype from which the immunogen was derived. That is, use of the immunogen can generate antibodies that specifically bind HIV-1 Env proteins and HIV-1 Env trimeric complexes from more than one clade. An immunogen can be an HIV-1 Env protein, fragment thereof, HIV-1 Env trimeric complex, or portion thereof described herein. An immunogen can also be a nucleic acid molecule, vector, host cell, or pharmaceutical composition described herein.

In an embodiment, antibodies neutralize HIV-1 or broadly neutralize HIV-1. Broadly neutralizing Abs (BnAbs) can neutralize infection of a large spectrum of genetically diverse HIV-1 viruses. BnAbs can reduce the infectious titer of HIV-1 by binding to and inhibiting the function of related HIV-1 Env antigens. Related HIV-1 Env antigens share at least about 85%, 90%, 95%, 96%, 97%, 98%, 99% or more sequence identity with an antigenic surface HIV-1 Env. In some embodiments, broadly neutralizing antibodies to HIV-1 Env are distinct from neutralizing antibodies to HIV-1 Env in that they neutralize with an ID₅₀>40 a high percentage (e.g., about 50%, 60%, 70%, 80% or more) of the many types of HIV-1 in circulation. In an embodiment, a BnAb can neutralize the function of HIV-1 Env from more than one clade. Therefore, broadly neutralizing antibodies to HIV-1 Env are distinct from other antibodies to HIV-1 Env in that they neutralize a high percentage of the many types of HIV in circulation.

Examples of broadly neutralizing antibodies include b12 and VRC01, which bind to the CD4 binding site of gp120; 2F5 and 4E10, which bind to the membrane-proximal external region (MPER) of gp41; and PG9 and PG16, which bind to the V1V2 domains of the trimer. Other examples include PGT122 and 35022.

Methods to assay for neutralization activity are well known in the art and include, but are not limited to, plaque reduction neutralization (PRNT) assays, microneutralization assays, flow cytometry based assays, single-cycle infection assays (see e.g., Martin et al. (2003) Nature Biotechnology 21:71-76), and pseudovirus neutralization assays (see e.g., Georgiev et al. (Science, 340, 751-756, 2013), Seaman et al. (J. Virol., 84, 1439-1452, 2005), Mascola et al. (J. Virol., 79, 10103-10107, 2005).

An antibody can be made in vivo in suitable laboratory animals or in vitro using recombinant DNA techniques. Means for preparing and characterizing antibodies are well known in the art. See, e.g., Dean, Methods Mol. Biol. 80:23-37 (1998); Dean, Methods Mol. Biol. 32:361-79 (1994); Baileg, Methods Mol. Biol. 32:381-88 (1994); Gullick, Methods Mol. Biol. 32:389-99 (1994); Drenckhahn et al. Methods Cell. Biol. 37:7-56 (1993); Morrison, Ann. Rev. Immunol. 10:239-65 (1992); Wright et al. Crit. Rev. Immunol. 12:125-68 (1992).

“Specifically binds” or “specific for” means that a first antigen, e.g., HIV-1 Env, a portion thereof, a HIV-1 Env trimeric complex or portions thereof recognizes and binds to an antibody or antigen binding fragment thereof with greater affinity than other non-specific molecules. A non-specific molecule is an antigen that shares no common epitope with the first antigen. In embodiments a non-specific molecule is not an HIV-1 Env and is not related to HIV-1 Env. For example, an antibody raised against a first antigen (e.g., HIV-1 Env) to which it binds more efficiently than to a non-specific antigen can be described as specifically binding to the first antigen. In embodiments an antibody or antigen-binding fragment thereof specifically binds to an HIV-1 Env, HIV-1 Env trimeric complex, or portion thereof when it binds with a binding affinity Kₐ of 10⁷ l/mol or more. An antibody or binding fragment thereof can specifically bind to an HIV-1 Env protein, portion thereof, HIV-1 Env trimeric complex or portion thereof when the interaction has a K_{D} of less than 10⁻⁶ Molar, such as less than 10⁻⁸ Molar, less than 10⁻⁸ Molar, less than 10⁻⁹, or even less than 10⁻¹⁰ Molar.

In an embodiment an antibody or antigen binding fragment can specifically bind to HIV-1 Env or trimeric complexes of HIV-1 Env from two or more clades.

Specific binding can be tested using, for example, an enzyme-linked immunosorbant assay (ELISA), a radioimmunoassay (RIA), or a western blot assay using methodology well known in the art.

In an embodiment an antibody or fragment thereof specifically binds to an HIV-1 Env protein, portion thereof, HIV-1 Env trimeric complex or portion thereof in the presence of a heterogeneous population of proteins and other biologics. Thus, an antibody or fragment thereof specifically binds to a particular target HIV-1 Env protein and does not bind in a significant amount to other proteins or polysaccharides present in the sample or subject.

An embodiment provides a method of treatment of HIV-1 related disease or symptom thereof comprising administrating one or more of the neutralizing or broadly neutralizing antibodies described above to a subject (e.g., a mammal such as a human).

An embodiment provides a method of enhancing the binding of an antibody to an HIV-1 Env trimeric complex or portion thereof. The method comprises making one or more amino acid mutations described herein to one or more HIV-1 Env proteins such that the HIV-1 Env trimeric complex or portion thereof comprises one or more of the mutations. An antibody specific for HIV-1 Env is contacted with the HIV-1 Env trimeric complex or portion thereof comprising one or more of the mutations. Binding of the antibody is enhanced as compared to binding of the antibody to an HIV-1 Env trimeric complex or portion thereof that does not comprise one or more of the mutations described herein.

### Methods of Making, Screening, and Identifying Antibodies

An embodiment provides methods for isolating antibodies that specifically bind to a HIV-1 Env protein or fragment thereof comprising one or more of the mutations described herein or an HIV-1 trimeric complex or portion thereof comprising one or more of the mutations described herein. An effective amount of an immunogen is administered to a subject, such as a mammal and antibodies are isolated. An effective amount is an amount sufficient to elicit antibodies to the HIV-1 Env immunogen.

An embodiment provides a method of identifying antibodies that specifically bind to an HIV-1 Env protein or fragment thereof comprising one or more of the mutations described herein or an HIV-1 Env trimeric complex or fragment thereof comprising one or more of the mutations described herein. Methods comprise, for example, administering an effective amount of an immunogen to B cells in an in vitro cell culture system to generate antibodies that specifically bind to the HIV-1 Env protein or the HIV-1 Env trimeric complex and isolating antibodies specific for the administered complex or composition. An effective amount is an amount sufficient to generate antibodies to the HIV-1 Env immunogen.

An embodiment provides a method of making or screening for an isolated hybridoma that produces a broadly neutralizing antibody that specifically binds to an HIV-1 Env protein or fragment thereof comprising one or more of the mutations described herein or an HIV-Env trimeric complex or portion thereof comprising one or more of the mutations described herein. The method comprises immunizing a mammal with an effective amount of an immunogen as described herein and isolating splenocytes from the immunized mammal. An effective amount is an amount sufficient to elicit antibodies to the HIV-1 Env immunogen. The isolated splenocytes are fused with an immortalized cell line to form hybridomas. Individual hybridomas are screened for production of an antibody that specifically binds with said trimeric complex or protein thereof to isolate the hybridoma.

In all methods described above, an immunogen can be, for example, an HIV-1 Env trimeric complex or portion thereof comprising one or more of the mutations described herein; an HIV-1 Env protein or fragment thereof comprising one or more mutations described herein; a nucleic acid molecule, vector, or host cell encoding or capable of expressing an HIV-1 Env protein, fragment thereof, HIV-1 Env trimeric complex or portion thereof comprising one or more of the mutations described herein.

The compositions and methods are more particularly described below and the Examples set forth herein are intended as illustrative only, as numerous modifications and variations therein will be apparent to those skilled in the art. As used in the description herein and throughout the claims that follow, the meaning of “a”, “an”, and “the” includes plural reference unless the context clearly dictates otherwise. The term “about” in association with a numerical value means that the value varies up or down by 5%. For example, for a value of about 100, means 95 to 105 (or any value between 95 and 105).

The terms used in the specification generally have their ordinary meanings in the art, within the context of the compositions and methods described herein, and in the specific context where each term is used. Some terms have been more specifically defined below to provide additional guidance to the practitioner regarding the description of the compositions and methods.

All patents, patent applications, and other scientific or technical writings referred to anywhere herein are incorporated by reference herein in their entirety. The embodiments illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the aspects herein.

In addition, where features or aspects of the invention are described in terms of Markush groups or other grouping of alternatives, those skilled in the art will recognize that the invention is also thereby described in terms of any individual member or subgroup of members of the Markush group or other group.

The following are provided for exemplification purposes only and are not intended to limit the scope of the invention described in broad terms above.

### EXAMPLES

### Example 1: Methods

A. Tissue Culture

A derivative of Expi293F cells was used in which CXCR4 expression was knocked out⁴⁷; these cells do not express CD4, CCR5, or CXCR4 receptors. Cells were cultured in Expi293 Expression Medium (Life Technologies), 8% CO₂, 37° C., at 125 rpm, and transfected with Expifectamine (Life Technologies). Unless otherwise stated, for testing of targeted mutants, 500 ng plasmid DNA was transfected per 2×10⁶/mL of cells, and ExpiFectamine Transfection Enhancers 1 and 2 (Life Technologies) were added 18 h later. Cells were analyzed 24-30 h post-transfection. For transfecting libraries such that, on average, cells acquire no more than one coding sequence⁴⁷, 1 ng/ml library DNA was diluted with 1.5 μg/ml pCEP4-ΔCMV carrier DNA, and transfected using Expifectamine into CXCR4-knockout Expi293F cells at a density of 2×10⁶ cells/ml. The medium was replaced 2 h post-transfection, and cells were prepared for sorting 24-26 h post-transfection. The pCEP4-ΔCMV carrier plasmid was generated by digesting pCEP4 with Sail and ligating the vector backbone back together, effectively removing the CMV promoter, multiple cloning site, and SV40 polyadenylation sequence, but maintaining the EBNA1 gene and oriP replication origin.

B. BaL gp160 Library Generation

A synthetic, codon-optimized Env gene from the BaL HIV-1 isolate (GenBank Accession No. AAA44191.1) was generated from gBlocks (Integrated DNA Technologies) and cloned into the Nhel-XhoI sites of pCEP4 (Invitrogen). The gene encodes Env_{BaL} residues E31-L856 (numbering based on the HXB2 reference strain) fused to an N-terminal CD5 leader peptide (sequence MPMGSLQPLATLYLLGMLVASVLA). When transfecting cells under conditions that yielded a single coding variant per cell, a pCEP4 derivative vector (pCEP4-intron) containing a strong 5′ chimeric intron was used for enhanced expression. This was created by cloning the intron from plasmid pRL-SV40 (Promega) into the Kpnl-Nhel sites of pCEP4. These plasmids are deposited with Addgene.

Single site-saturation mutagenesis (SSM) libraries were generated by overlap extension PCR⁷⁶. Three separate SSM libraries were constructed focused on the Env N-terminus (a.a. 31-265; Library A), center (a.a. 266-529; Library B), and C-terminus (a.a. 530-856; Library C). The PCR products were cloned by restriction enzyme digestion and ligation into the Nhel-Bglll (Library A), BamHI-NotI (Library B), and PstI-XhoI (Library C) sites of BaLgp160 inserted into the Nhel-XhoI sites of pcDNA3.1(+) (Invitrogen), with the vector PstI and BgIII sites removed by QuikChange (Agilent) mutagenesis. Ligations were transformed into NEB 5-α electrocompetent E. coli (New England Biolabs), and plasmid DNA for each library was prepared using GeneJET Maxiprep Kit (Thermo Scientific). Following library construction in the pcDNA3.1(+) vector, the full-length diversified BaLgp160 library inserts was subcloned into the Nhel-XhoI sites of pCEP4-intron. At all cloning steps, the number of transformants was at least an order of magnitude greater than the possible library diversity. Combined, the three BaL gp160 SSM libraries covered 16,332 out of 16,520 possible single amino acid mutations, based on a minimum frequency of 5.7×10⁻⁶ (corresponding to approximately 10 reads) in the deep sequenced plasmid libraries.

C. DU422 gp140 Library Construction

A synthetic, codon-optimized gp160 gene from the DU422 HIV-1 isolate (GenBank Accession No. ABD83641.1) was cloned into the Nhel-XhoI sites of pCEP4 (Invitrogen). This plasmid is deposited with Addgene (#100926). Using PCR-based assembly, the gp140 ectodomain (a.a. N31-N677, HXB2 reference numbering) was fused to a C-terminal gly/ser-rich linker, 6his tag, and the transmembrane helix of HLA class I α chain for surface display, and inserted into the Nhel-XhoI sites of pCEP4.

Overlap extension PCR was used to create the SSM libraries⁷⁶. Three separate SSM libraries were constructed focused on the gp140_{DU422} N-terminus (a.a. 31-279; NT library), center (a.a. 280-577; central library), and C-terminus (a.a. 578-677; CT library). Mutagenized PCR segments were ligated into the Nhel-Pfl23II (NT library), SbfI-HindIII (central library), or Pfl23II-Xho1 (CT library) sites of pCEP4-gp140_{DU422}. Ligations were electroporated into NEB 5-β E. coli (New England Biolabs), and library plasmid DNA was purified using a GeneJET Maxiprep Kit (Thermo Scientific). Based on a minimum frequency of 5×10⁻⁶, all possible 12,820 single amino acid substitutions were present in the three SSM libraries.

D. Sorting BaL gp160 Libraries for Binding to sCD4, VRC01 and PG16

To evolve Env variants for high binding signals to soluble CD4 (sCD4), cells were harvested 24-26 hours following transfection with library DNA as described above. Cells were centrifuged at 500 g for 1 min at 4° C., the pellets were washed with cold phosphate buffered saline supplemented with 0.2% BSA (PBS-BSA), and incubated on ice for 40 minutes with 200 nM sCD4-183 (domains D1-D2). Soluble recombinant sCD4-183 was obtained through the NIH AIDS Reagent Program, Division of AIDS, NIAID, from Progenics. After incubation, cells were washed twice with cold PBS-BSA, incubated on ice for 30 minutes with FITC-conjugated anti-CD4 (clone M-T441, LifeSpan BioSciences, 1/200 dilution), washed twice, and resuspended in PBS-BSA.

To evolve the Env libraries for high binding signals to VRC01 or PG16, cells were washed with cold PBS-BSA 24-26 hours post-transfection, incubated on ice for 40 minutes with either 5 nM VRC01 (obtained through the NIH AIDS Reagent Program from John Mascola)⁴⁴ or 2 nM PG16 (obtained through the NIH AIDS Reagent Program)³⁹, washed twice, incubated on ice for 30 minutes with APC-conjugated anti-human IgG Fc antibody (BioLegend, clone HP6017, 1/300 dilution), washed twice more, and resuspended in PBS-BSA.

Labeled cells were sorted on a BD FACS Aria II at the Roy J. Carver Biotechnology Center. Immediately prior to sorting, 1 μg/ml propidium iodide was added to the cells, and dead cells that were propidium iodide-positive were removed during gating. Auto-fluorescent cells in either the APC channel (after staining with FITC-conjugated anti-CD4) or FITC channel (after staining with APC-conjugated anti-IgG Fc) were also removed during gating. In the sort gates for sCD4- or VRC01-bound cells, the highest 0.5% (Libraries A and B) and 1.0% (Library C) of FITC- or APC-positive cells were collected, respectively. In the sort gates for PG16-bound cells, the highest 0.6% (Libraries A and B) and 0.9% (Library C) of APC-positive cells were collected. These gated cell populations in the respective libraries had similar binding signals. Sort conditions are listed in Table 1. Sorted cells were collected by centrifugation (400 g, 3 min, 4° C.) and frozen at −80° C. To maintain cell viability and mRNA quality during the experiment, samples were sorted for a maximum of 4 hours into tubes that had been coated overnight with fetal bovine serum. To collect greater numbers of cells than one 4-hour sort provided, libraries were prepared again and frozen sorted cell pellets from multiple days' experiments were pooled during RNA extraction. Each replicate typically required 8 hours of sorting per library.

E. FACS-Based Selection of the Cell Libraries Expressing DU422 gp140

Recombinant sCD4-183 (provided by Progenics) and PG16³⁹ were obtained from the NIH AIDS Reagent Program. Division of AIDS. NIAID. To select gp140_{DU422} variants based on sCD4 binding, cells were centrifuged at 500 g, 2 min, 4° C., 24-26 h post-transfection. Cells were washed with cold phosphate buffered saline supplemented with 0.2% BSA (PBS-BSA), incubated on ice for 40 minutes with 10 nM sCD4 in PBS-BSA, washed twice, incubated on ice for 30 minutes with fluorescein isothiocyanate (FITC)-conjugated anti-CD4 (clone M-T441, LifeSpan BioSciences, 1/200 dilution), washed twice, and resuspended in PBS-BSA. To select gp140_{DU422} variants based on PG16 binding, transfected cells were washed with cold PBS-BSA, incubated on ice for 40 minutes with 3 nM PG16, washed twice, incubated for 30 minutes with allophycocyanin (APC)-conjugated anti-human IgG Fc antibody (BioLegend, clone HP6017, 1/250 dilution), washed twice more, and resuspended in PBS-BSA.

Labeled cells were sorted on a BD FACS Aria II at the Roy J. Carver Biotechnology Center, University of Illinois. Single cells were gated based on FSC/SSC, and dead cells that were positive for propidium iodide (added to a final concentration of 1 μg/ml) were excluded. Autofluorescent cells in the APC channel (after staining with FITC-conjugated anti-CD4) or FITC channel (after staining with APC-conjugated anti-IgG Fc) were also excluded. In the final sort gates, the highest 0.3% (NT library) or 0.4% (central and CT libraries) of FITC-positive cells were collected during the sCD4 binding selections. For sorting PG16-bound cells, the highest 0.6% of APC-positive cells were collected. Sorted cells were centrifuged at 400 g, 3 min, 4° C., and pellets were frozen at −80° C. To maintain cell viability and mRNA quality, samples were sorted for a maximum of 4 h into tubes that had been coated overnight with fetal bovine serum. To collect greater numbers of cells than one sort provided (a typical replicate required 6-8 h of sort time), libraries were freshly prepared and frozen cell pellets from multiple sort runs were pooled during RNA extraction. Sorting conditions are summarized in Table 6.

F. Deep Sequencing the BaL gp160 Libraries

Total RNA was extracted from sorted cells using a GeneJET RNA Purification Kit (Thermo Scientific), and first strand cDNA was synthesized with high-fidelity AccuScript reverse transcriptase (Agilent Technologies) primed with oligonucleotides that annealed downstream of the diversified regions: BALgp160_libA_RT_rev (5′-CGTTCAGCTGAACAATGATG-3) (SEQ ID NO:4) annealing to a.a. 284-289 of Library A, BALgp160_libB_RT_rev (5′-TGTTGGACAATACCAGACAG-3) (SEQ ID NO:5) annealing to a.a. 545-550 of Library B, and the EBV-Reverse sequencing primer annealing to the 3′-UTR for Library C. To generate fragments for deep sequencing, the cDNA was PCR amplified in two rounds. In the first round (18 thermocycles), primer overhangs added complementary sequences to the Illumina sequencing primers. In the second round (15 thermocycles), primer overhangs added barcodes and adaptor sequences for annealing to the Illumina flow cell. Thermocycling was kept to a minimum to reduce the introduction of PCR biases and errors. Each of the gp160 libraries was amplified as three overlapping fragments to achieve full sequencing coverage. DNA was sequenced at the UIUC Roy J. Carver Biotechnology Center on an Illumina MiSeq v3 (2×300nt kit) or HiSeq 2500 (2×250nt kit).

G. Deep Sequencing the DU422 gp140 Libraries

Total RNA was extracted from sorted cells using a GeneJET RNA Purification Kit (Thermo Scientific). For the central and CT libraries, first strand cDNA was synthesized with high-fidelity AccuScript (Agilent Technologies) primed with a gene-specific oligonucleotide and the EBV-Reverse sequencing primer (5′-GTGGTTTGTCCAAACTCATC-3′ (SEQ ID NO:6); anneals to the 3′-UTR). For the NT library, cDNA was reverse transcribed with SuperScript IV Vilo Master Mix (Thermo Scientific). The cDNA was PCR amplified in two rounds to generate fragments for Illumina sequencing. In the first round (18 thermocycles), primer overhangs added complementary sequences to the Illumina sequencing primers. In the second round (9 thermocycles for the NT library, and 15 thermocycles for the central and CT libraries), primer overhangs added barcodes and adaptor sequences for annealing to the Illumina flow cell. Thermocycling was minimized to reduce PCR biases and errors. The NT and central gp140 libraries were each amplified as two overlapping fragments to achieve full sequencing coverage. DNA was sequenced at the UIUC Roy J. Carver Biotechnology Center on an Illumina HiSeq 2500 (2×250nt kit).

H. Deep Sequencing Analysis

Deep sequencing data were analyzed with Enrich⁷⁷. Log₂ enrichment ratios of mutants were normalized by subtracting the enrichment of the wildtype sequence. Commands are available in the data deposition with NCBI's Gene Expression Omnibus⁷⁸ under series accession number GSE102276.

I. Directed Evolution of an Env_{BaL} Combinatorial Library

The ectodomain of Env_{BaL} (a.a. E31-K677) was cloned downstream of a CD5 leader peptide and upstream from a gly/ser-rich linker fused to a 6his tag and the TM helix of HLA class I α chain for surface display. A combinatorial library, containing core mutations enriched in the sequence-activity landscape for PG16 binding, was synthesized by oligo assembly and cloned into the Nhel-XhoI sites of pCEP4-intron. Expi293F CXCR4-KO cells (2×10⁵ cells/ml) were transfected using Expifectamine with 1 ng/ml library DNA and 1.5 μg/ml pCEP4-ΔCMV. Cells were harvested 24-26 h post-transfection, and stained with PG16 as described for the SSM library selection. Cells with the highest APC fluorescence signal (top 0.3%) were collected on a BD FACS Aria II sorter and frozen at −80° C. Total RNA was extracted from the frozen cell pellet using a GeneJET RNA Purification Kit (Thermo Scientific), and first strand cDNA was synthesized with SuperScript IV VILO Master Mix (Thermo Scientific) reverse transcriptase. The Env insert was PCR-amplified from the cDNA using Phusion (Thermo Scientific), and re-cloned into pCEP4-intron for another round of enrichment. The numbers of transformants during cloning steps were orders of magnitude greater than the possible library diversity of 9.216 variants, ensuring that all possible mutant combinations in the library were adequately sampled. The library selection was repeated three times. To increase stringency, the PG16 concentration was decreased from 2 nM (sort 1; 21,500 cells collected) to 1 nM (sort 2; 39,100 cells) to 0.5 nM (sort 3; 31,600 cells). After the third round of directed evolution, plasmid DNA from individual clones was purified and tested.

J. Env_{BaL} Mutants Binding to Antibodies

Highly enriched Env_{BaL} mutants identified in both replicate evolution experiments for PG16 binding were transfected into Expi293F CXCR4-knockout cells. Transfected cells were washed with cold PBS-BSA 24 h post-transfection, incubated on ice for 40 minutes with PG16 (2 nM or 0.5 nM), washed twice, incubated on ice for 30 minutes with APC-conjugated anti-human IgG Fc antibody (BioLegend, clone HP6017, 1/300 dilution), washed twice and analyzed on a BD LSR II flow cytometer. For titrating different antibodies, a 1:3 serial dilution of the antibody was prepared in a 96-well round-bottomed plate. Cells were incubated with the antibody at 4° C. on a rocker and washed in the 96-well plate as described above. 39F^{79,80} was provided by the NIH AIDS Reagent Program from Dr. James E. Robinson. 268-D IV⁸¹, 2442⁸² and 3074^{83,84} were provided by Dr. Susan Zolla-Pazner through the NIH AIDS Reagent Program.

K. Env_{BaL} Glycosylation Site Mutants Binding to sCD4 and VRC01

Transfected Env_{BaL} (WT, N262Q, S264T, and S264A) cells were washed with cold PBS-BSA 24 h post-transfection. 1:3 Serial dilutions of sCD4 and VRC01 in PBS-BSA were prepared in 96-well round-bottomed trays. The washed cells were incubated with the ligands at 4° C. on a rocker for 40 minutes, washed twice, incubated for 30 minutes with secondary antibody (1/200 FITC-anti-CD4 clone M-T441 from LifeSpan BioSciences, or 1/300 APC-anti-IgG clone HP6017 from BioLegend), washed twice, and analyzed on a BD LSR II flow cytometer.

L. Env_{YU2}, Env₂₅₇₁₁, Env_{DU422}, Env_{Q769.d12} and Env_{Q642.d12} Binding to Antibodies and sCD4

Human codon-optimized Env sequences from HIV-1 strains YU2, 25711, DU422, Q769.d22 and Q842.d12 were cloned into the Nhel-XhoI sites of pCEP4 from gBlocks (Integrated DNA Technologies). In all cases, the native signal peptides were substituted with the strong CD5 leader sequence. These plasmids are deposited with Addgene. Mutations were made by overlap extension PCR.

Expi293F CXCR4-knockout cells were transfected at a density of 2×10⁶/ml, with 1 ml of cells per well of a 12-well tissue culture treated plate. Cells were transfected with Expifectamine (Life Technologies) using 1000 ng DNA per well. Eighteen hours post-transfection ExpiFectamine Transfection Enhancers 1 and 2 (Life Technologies) were added. Cells were harvested 42 h post-transfection, washed with cold PBS-BSA, incubated on ice for 40 minutes with PG16 at the indicated concentrations, washed twice, incubated on ice for 30 minutes with APC-anti-human IgG Fc antibody (BioLegend, clone HP6017, 1/300 dilution), washed twice, and analyzed on a BD LSR II flow cytometer. For titration curves with different antibodies, 1:3 serial dilutions of the antibody were prepared and binding was assayed in 96-well round-bottomed plates. PGT121⁵⁸, PGT128⁶⁸, and PGT145⁶⁸ were provided by IAVI through the NIH AIDS Reagent Program.

For binding assays to soluble CD4, cells were transfected as just described and collected 42 h post-transfection. Cells were washed with cold PBS-BSA, incubated on ice for 40 minutes with 1:3 serial dilutions of sCD4-183 prepared in 96-well round-bottomed plates, washed twice, incubated on ice for 30 minutes with FITC-anti-CD4 antibody (clone M-T441, LifeSpan BioSciences, 1/200 dilution), washed twice, and analyzed on a BD LSR II flow cytometer.

M. Structural Modeling

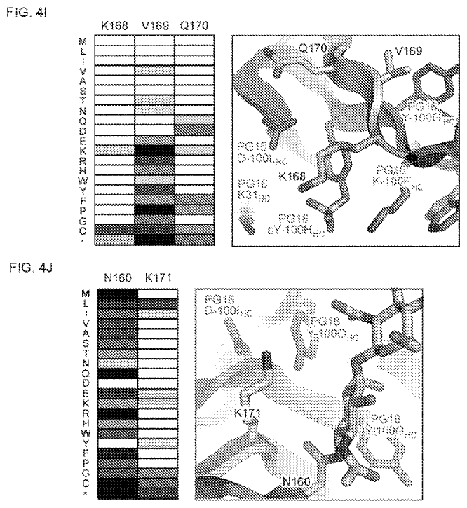

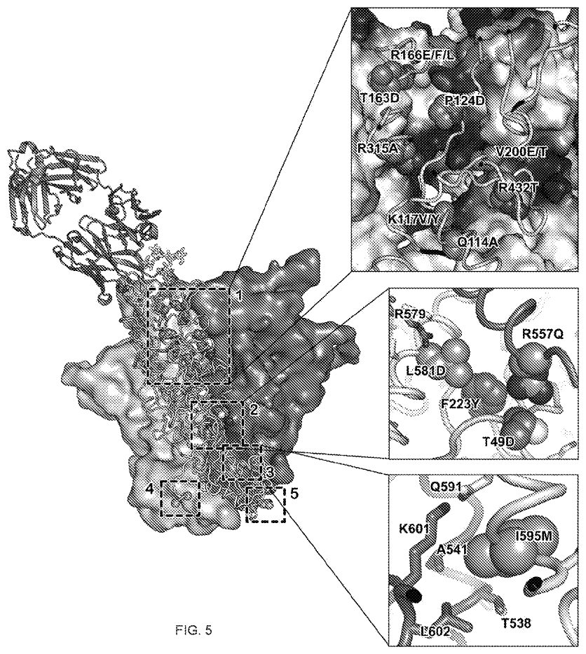

Homology modeling of BaL Env was based on crystal structures of sequences from other HIV-1 strains. VRC01-bound gp140_{BaL} in the closed conformation was modeled by threading the BaL strain sequence onto PDB 5FYK⁸⁵ with glycans removed, and rebuilding missing loops and minimizing side chain conformations in FoldIt⁸⁶. CD4-bound gp120_{BaL}, was generated by threading the BaL sequence onto PDB 1GC1⁵⁶, and minimizing side chain and backbone conformations with FoldIt. For modeling open-state gp140_{BaL} bound to CD4, the sequence of BaL Env was threaded onto PDB 5VN3¹³, which was then minimized with C3 symmetry using ROSETTA⁸⁷. The model of the PG16-bound apical epitope was generated by threading the BaL sequence onto PDB 4DQO⁶⁰. Coordinates for the glycan on N160 were kept, and a single N-acetyl-D-glucosamine sugar was added to N156. The structure in FIG. 5 was generated by superimposing the model of the PG16-bound epitope to the model of closed Env.

Homology modeling of DU422 Env was based on the crystal structures of JR-FL SOSIP.664 (PDB 5FYK⁸⁵) and CD4-bound B41 SOSIP.664 (PDB 5VN3¹³). Glycans were removed, the DU422 sequence was threaded on, and missing loops were rebuilt using FoldIt⁸⁶. Side chain and backbone conformations were then minimized with C₃ symmetry imposed around the trimer axis using xml scripting in ROSETTA⁸⁷. Point substitutions were modeled using FoldIt with local side chain minimization. Images were rendered with the PyMOL Molecular Graphics System, Schrödinger, LLC.

N. Western Blot

Cells transfected with BaL gp160 (WT, QES01, or QES02) were harvested, centrifuged and resuspended in reducing SDS dye. Sonicated samples were run on a 4-20% gradient polyacrylamide gel (Bio-Rad). Gels were transferred to PVDF membrane using a BioRad Mini Trans-Blot apparatus. Blots were blocked in TBST (Tris-buffered saline, 0.1% TWEEN® polysorbate 20) containing 3% BSA, washed with TBST, stained with goat polydonal anti-HIV gp160 (1/500: Abcam ab117122) for 30 minutes at room temperature, washed in TBST, stained with donkey ant-goat IgG H&L alkaline phosphatase-conjugate (1/1000; Abcam ab97112) for 30 minutes at room temperature, washed and visualized with Thermo Scientific 1-Step NBT/BCIP Substrate Solution.

O. Syncytia Formation Assay

Expi293F CXCR4-knockout cells (2×10⁶ cells/ml) were co-transfected using Expifectamine with untagged CD4 (50 ng/ml pCMV3-CD4, Sino Biological HG10400-UT) plus N-terminal myc-tagged CCR5 (450 ng/ml pCEP4-myc-CCR5). Partner cells for fusion were transfected with 500 ng/ml of pCEP4 plasmid encoding Env. For controls, the plasmid was replaced with empty vector. Five hours post-transfedion 0.2×10⁶ receptor- and Env-expressing cells were mixed with 600 μl Expi293 Expression Medium in a poly-L-lysine-coated well of a 12-well tissue culture tray. Wells had been previously coated with 0.01% poly-L-lysine (Sigma) for 30 minutes at room temperature, and washed with water and Expi293 Expression Medium. Plates were incubated with no agitation at 37° C., 8% CO₂.

For quantitation by flow cytometry, wells were washed 20 h later with warm PBS, cells were detached for 15 minutes at 37° C. with 0.25% trypsin-2.21 mM EDTA, washed with cold PBS-BSA, and analyzed on a BD LSR II flow cytometer. The positive gate was set at 1% of untransfected cells for high FSC/SSC events.

For qualitative microscopic observation, cells were stained 20 h later with 0.06 μM calcein-AM (BioLegend) and NucBlue Fixed Cell Stain (Thermo Scientific) for ˜10 minutes. Medium was replaced, and cells were visualized on a Leica DMi8 inverted microscope. Image overlays were created in Fiji ImageJ.

M. Protein Purification and Size Exclusion Chromatography

To express gp120 subunits, residues E31-K510 (BaL), A31-K510 (Q769.d22), and V31-K510 (Q842.d12) were cloned downstream of a CD5 leader peptide, and fused at their C-termini to a linker (AGG) and 8-his tag. The Env_{BaL} gp140 construct encoded the soluble extracellular region upstream of the MPER (residues E31-D664) with a CD5 leader peptide, and fused at the C-terminus to a linker (GSGSGGSG) (SEQ ID NO:3) and 8-his tag. These coding sequences were cloned into the Nhel-XhoI sites of pcDNA3.1 (+) (Invitrogen).

Plasmids were transfected into Expi293F cultures using a protocol adapted from⁸⁸. For each milliliter of culture, 1 μg DNA was mixed with 5 μg linearized polyethyleneimine (MW 25,000; Polysciences) in 100 μl of OptiMEM (Gibco). The mixture was incubated for 20 minutes at room temperature, then added to the cell culture at a density of 2×10⁶/ml. Expi293 Transfection Enhancers (Life Technologies) were added 18 h post-transfection. Cells were centrifuged (1200×g, 15 minutes) 4 days post-transfection, and secreted protein was purified from the culture supernatant.

Protein was purified at 4° C. The supernatant was dialyzed against 20 mM Tris pH 8.0/225 mM NaCl for 6-8 h, followed by dialysis overnight against 20 mM Tris pH 8.0/20 mM imidazole/300 mM NaCl. Equilibrated NiNTA (50% slurry, 500 μl per 40 ml culture, Thermo Scientific) was incubated with the sample for 1 h on a rocker, collected in a gravity column, and washed with 20 ml of purification buffer (20 mM Tris pH 8.0/300 mM NaCl) containing 20 mM imidazole. Protein was eluted using a step gradient of purification buffer containing 50, 100 and 250 mM imidazole (1 ml per fraction). The 100 and 250 mM imidazole fractions were found to contain 8-his-tagged gp120 or gp140 based on SDS-PAGE analysis, and were pooled and concentrated using a 30 kD MWCO centrifugal device (Sartorius). Samples were separated by size exclusion chromatography using a Superose 6 increase 10/300 GL column on an ÄKTA pure system (GE Healthcare) with PBS as the running buffer.

N. BG505 SOSIP.664 Mutant Screening, Purification and ELISA Analysis

A codon-optimized gene fragment of BG505 SOSIP.664 (T332N) was cloned into the Nhel-XhoI sites of pCEP4. A CD5 leader peptide was placed at the N-terminus, and the sequence was fused C-terminally via a gly/ser-rich linker to a 6his tag followed by the TM helix of HLA class I α chain for tethering to the cell membrane. Mutations were made by overlap extension PCR and screened for PG16 binding in transfected Expi293F CXCR4-KO cells as described above.

For purification, BG505 SOSIP.664 (T332N) was subcloned into pCEP4 (Nhel-XhoI sites) with the C-terminal TM tether replaced by gly/ser-rich linker and 8his tag. The protein was expressed and purified as described above.

For PG16 binding analysis by ELISA, purified BG505 SOSIP protein (50 μl per well at 2 μg/ml in PBS) was incubated for 1 h at room temperature in a copper-coated 96-well plate (Thermo Scientific). Wells were washed 6 times with PBS containing 0.2% TWEEN® polysorbate 20 (PBS-T1), blocked with 100 μl 5% skim milk in PBS-T for 30 min, and then incubated for 2 h with 50 μl PG16 in PBS-T1 containing 2% skim milk. Wells were washed 6 times with PBS-T1, incubated for 1 h with peroxidase-conjugated donkey anti-human IgG-Fc (50 μl of a 1:15000 dilution in PBS-T1; Jackson ImmunoResearch Laboratories), and washed another 6 times with PBS-T1. For VRC01 binding analysis, BG505 SOSIP (50 μl per well at 2 μg/ml in PBS) was incubated for 1 h at room temperature in a copper-coated 96-well plate. Wells were washed 6 times with PBS containing 0.00005% TWEEN® polysorbate 20 (PBS-T2), blocked with 100 μl 3% BSA in PBS for 15 min, and then incubated for 2 h with 50 μl VRC01 in PBS containing 1.2% BSA. Wells were washed 6 times with PBS-T2, incubated for 1 h with peroxidase-conjugated donkey anti-human IgG-Fc (100 μl of a 1:5000 dilution in PBS), and washed another 6 times with PBS-T2. For CD4-IgG2 binding analysis, BG505 SOSIP (50 μl per well at 2 μg/ml in TBS: 50 mM Tris-CI pH 7.5, 150 mM NaCl) was incubated for 1 h at room temperature in a nickel-coated 96-well plate (Thermo Scientific). Wells were washed 5 times with TBS containing 0.05% TWEEN® polysorbate 20 (TBS-T), blocked with 100 μl TBS-T containing 3% BSA for 30 min, and then incubated for 2 h with 50 μl CD4-IgG2 (provided by Progenics Pharmaceuticals through the NIH AIDS Reagent Program) in TBS containing 1.2% BSA and 0.02% TWEEN® polysorbate 20. Wells were washed 5 times with TBS-T, incubated for 1 h with peroxidase-conjugated donkey anti-human IgG-Fc (100 μl of a 1:5000 dilution in TBS-T), and washed another 5 times with TBS-T. All ELISA plates were developed with 1-Step Ultra TMB-ELISA Substrate Solution (Thermo Scientific), and absorbance was measured at 630 nm.

P. Sequences

Native Env sequences with GenBank accession numbers from tested HIV-1 strains are shown below:

```
BaL Env (GenBank AAA44191.1)
(SEQ ID NO: 7)
    1 mrvteirksy qhwwrwgiml lgilmicnae eklwvtvyyg vpvwkeattt lfcasdrkay
  61 dtevhnvwat hacvptdpnp qevelknvte nfnmwknnmv eqmhediisl wdqslkpcvk
121 ltplcvtlnc tdlrnatngn dtnttsssrg mvgggemknc sfnittnirg kvqkeyalfy
181 kldiapidnn snnryrlisc ntsvitqacp kvsfepipih ycapagfail kckdkkfngk
241 gpctnvstvq cthgirpvvs tqlllngsla eeevvirsan fadnakviiv qlnesveinc
301 trpnnntrks ihigpgrafy ttgeiigdir qahcnlsrak wndtlnkivi klreqfgnkt
361 ivfkhssggd peivthsfnc ggeffycnst qlfnstwnvt eesnntvenn titlpcrikq
421 iinmwqevgr amyappirgq ircssnitgl lltrdggped nktevfrpgg gdmrdnwrse
481 lykykvvkie plgvaptkak rrvvqrekra vgigavflgf lgaagstmga aamtltvqar
541 lllsgivqqq nnllraieaq qhllqltvwg ikqlqarvla verylrdqql lgiwgcsgkl
601 icttavpwna swsnkslnki wdnmtwiewd reinnytsii yslieesqnq qekneqelle
661 ldkwaslwnw fditkwlwyi kifimivggl iglrivfsvl sivnrvrqgy splsfqthlp
721 ssrgpdrpgg ieeeggerdr drsgplvngf laliwvdlrs lflfsyhrlr dlllivmriv
781 ellglaggwe vlkywwnllq ywsqelknsa vsllnatava vaegtdrvie vlgravrail
841 hiprrirqgi erall
DU422 Env (GenBank ABD83641.1)
(SEQ ID NO: 8)
    1 mrvrgiprnw pqwwiwgilg fwmiiicrvv gnldlwvtvy ygvpvwkeak ttlfcasdak
  61 aydkevhnvw athacvptdp npqeivlenv tenfnmwknd mvdqmhedii slwdqslkpc
121 vkltplcvtl ncknvnisan anatatlnss mngeikncsf ntttelrdkk qkvyalfykp
181 dvvplnggeh netgeyilin cnsstitqac pkvsfdpipi hycapagyai lkcnnktfng
241 tgpcnnvstv qcthgikpvv stqlllngsl aeeeiivrse nltnniktii vhlnksveik
301 ctrpnnntrk svrigpgqtf yatgeiigdi reahcnisre twnstliqvk eklrehynkt
361 ikfepssggd levtthsfnc rgeffycdtt klfnetklfn eseyvdnkti ilpcrikqii
421 nmmqevgram yappiegnit cksnitglll twdggenste gvfrpgggnm kdnwrselyk
481 ykvveikplg vaptkskrkv vgrekravgl gavllgflga agstmgaasi tltvqarqll
541 sgivqqqsnl lraieaqqhl lqltvwgikq lqtrvlaier ylkdqqllgl wgcsgklica
601 tavpwnssws nkslgdiwdn mtwmqwdrei snytntifrl ledsqnqqek nekdllalds
661 wknlwnwfdi tnwlwyikif imivggligl riifgvlaiv krvrqgyspl sfqtlipnpr
721 gpdrlgriee eggeqdkdrs irlvsgflal awddlrslcl fsyhqlrdfi ltaaraaell
781 grsslrglqr gwevlkylgn lvqywglelk rsainlfdti aiavaegtdr iieviqricr
841 airyiptrir qgfeaall
25711 Env (GenBank ABL67448.1)
(SEQ ID NO: 9)
    1 mrvkgtrksy qqwwiwavlg fwmlmicnvg gnlwvtvyyg vpvwkeaktt lfcasdakgy
  61 dkevhnvwat hacvptdpnp qemplenvte nfnmwendmv nqmhedvisl wdeslkpcvk
121 ltplcvtlnc tdvnknvsss dtdnyketmk erknctfnmt telrdknqkk yalfykldiv
181 plddndnasy rlincntstl tqacpkvsfd pipihycapa gyailkcknk tfngigpcnk
241 vstvqcthgi kpvvstqlll ngslaeediv irsenitdna ktiivhlnes veivcirpnn
301 ntrksirigp gqtfyatgdi vgdirqaycn isegkwnktl qrvseklaeh fpnstinfns
361 ssggdleitt hsfncggeff ycntsglfng tymnndtksn dtksnsssii tipcrikqii
421 nmwqevgrav yappiagnit cksnitgill trdggrgeev kndtetfrpg ggnmkdnwrs
481 elykykvvei kplgvaptaa krrvverekr avglgavllg flgaagstmg aasitltvqa
541 rqllsgivqq qsnllraiea qqhmlqltvw gikqlqarvl aierylkdqq llgiwgcsgk
601 licttavpwn sswsnknqte iwdkmtwmqw dreisnytdt iyrlledsqn qqeknekdll
661 eldkwqnlws wfnitnwlwy irifimivgg liglriifav lsivnrvrqg ysplsfqtla
721 pnprgldrlg rieeeggked rnrsirlvhg flalawddlr slclfsyhrl rdlilliara
781 vellgqrgwe alkylagivq ywglelkksa vslfdtiaia vaertdriig liqgicraic
841 niprrirqgf eaalg
Q769.d22 Env (GenBank AAM66234.1)
(SEQ ID NO: 10)
    1 mramgiqrnw qnlwrwgtmi lgmiliccsaagnlwvtvyy gvpvwrdaet tlfcasdaka
  61 ydreahnvwa thacvptdps pqevplgnvt eefnmwknnm veqmhtdiis lwdqslqpcv
121 kltplcvtln csnsnnipsv snitddmkee ikncsfnmtt elkdkkqnvy slfyrldvvp
181 letktnqnss hsryrlincn tsaitqacpk vsfepipihy capagfailk cndkgfngtg
241 lcknvstvqc thgikpvvst glllngslae gkvmvrseni tnnakniiiq fnnsvqinct
301 rpgnntrksi hlgpgkvfya tdiigdirka hcnvnrqqwn ktlqdvatql rthfrnrtii
361 fnnslggdle itthsfncrg effycntsgl fngiwngtqe pnrtesndti tlqcrikqii
421 nmwqrvgqai yappiqgeir cesnitglil trdggiinst eetfrpgggd mrdnwrsely
481 kykvvkiepl gvaptkakrr vverekravg fgafflgflg aagstmgaas itltvqarql
541 lsgivqqqnn llraieaqqh llkltvwgik qlqarvlave rylkdqqllg iwgcsgkfic
601 tttvpwnssw snksqseiwd nmtwmqwdke innytqiiyd lieesqrqqe kneqdllald
661 kwanlwnwfd isnwlwyiki fimivgglig lriafavlsv inrvrqgysp lsfqthtpnp
721 rdldrpgrie eeggeqdrdr sirlvsgfla lawddlrslc lfsyhrlrdf ilvaartvel
781 lghislkglr rgweglkylg nllsywgrel kisainlldt iaivvaewtd riieigqrlc
841 raiiniprri rqgferall
Q842.d12 Env (GenBank AAM66242.1)
(SEQ ID NO: 11)
    1 mramgiqmnc qnlwrwgtmi lgmiifcsav dnlwvtvyyg vpvwkeaett lfcasdakay
  61 etekhnvwat hacvptdpnp geihlenvte efnmwknnmv eqmhtdiisl wdqslkpcvk
121 ltplcvtldc nnvtnngtsd mreeikncsf nmttelrdkr qkvyslfykl divqinedqg
181 nssnnkyrli tcntsaitqa cpkvtfepip ihycapagfa ilkckdeefn gigpcknvst
241 vqcthgikpv vstqlllngs laekevkirc enitnnakti ivqlvnpvki nctrpnnntr
301 ksihigpgqa fyatgdiigd irqahcnvnr tewnntlhqv veqlrkhfnk tinfanstgg
361 dleitthsfn cggeffycnt tnlfnstwnh tasmnstesn dtiilpcrik qiinmwqrvg
421 qamyappirg vircesnitg liltrdggnt nstretfrpg ggdmrdnwrs elykykvvki
481 eplgvaptka krrvverekr avgigavfig flgaagstmg aasitltvqa rqllsgivqq
541 qsnllraiea qqhllkltvw gikqlqarvl averylkdqq llgiwgcsgk licttsvpwn
601 sswsnksqne iwdnmtwlqw dkeisnytqi iydlleesqn qqekneqdll aldkwanlwn
661 wfdisnwlwy ikifimivgg liglrivfav lsvinrvrqg ysplsfqtht pnprgldrpe
721 rieeeggeqd knrsirlvsg flalawddlr slclfsyhrl rdfilivart vellghsslk
781 glrlgweglk ylgnllsywg relrisatnl ldtiaiviag wtdrvieigq rlcraflnip
844 rrirqgfera ll
```

The following are HIV-1 Env sequences as shown above (SEQ ID NOs:7-11) but with the N-terminal signal peptides replaced with a CD5 leader sequence (capitalized; MPMGSLQPLATLYLLGMLVASVL (SEQ ID NO:12) for better expression. Experiments were performed using these constructs:

CD5-BaL gp160
(SEQ ID NO: 13)
MPMGSLQPLATLYILGMLVASVLAeeklwvtvyygvpvwkeatttlfcasdrkaydtevhnvwathacvptdpnpqevelknvten
fnmwknnmveqmhediislwdqslkpcvkltplcvtlnctdlrnatngndtnttsssrgmvgggemkncsfnittnirgkvqkeya
lfykldiapidnnsnnryrliscntsvitqacpkvsfepipihycapagfailkckdkkfngkgpctnvstvqcthgirpvvstql
llngslaeeevvirsanfadnakviivqlnesveinctrpnnntrksihigpgrafyttgeiigdirqahcnlsrakwndtlnkiv
iklreqfgnktivfkhssggdpeivthsfncggeffycnstqlfnstwnvteesnntvenntitlpcrikqiinmwqevgramyap
pirgqircssnitgllltrdggpednktevfrpgggdmrdnwrselykykvvkieplgvaptkakrrvvqrekravgigavflgfl
gaagstmgaaamtltvqarlllsgivqqqnnllraieaqqhllqltvwgikqlqarvlaverylrdqqllgiwgcsgklicttavp
wnaswsnkslnkiwdnmtwiewdreinnytsiiyslieesqnqqekneqelleldkwaslwnwfditkwlwyikifimivggligl
rivfsvisivnrvrqgysplsfqthlpssrgpdrpggieeeggerdrdrsgplvngflaliwvdlrslflfsyhrlrdlllivmri
vellglaggwevlkywwnllqywsqelknsavsllnatavavaegtdrvievlqravrailhiprrirqglerall
CD5-DU422 gp160
(SEQ ID NO: 14)
MPMGSLQPLATLYLLGMLVASVLAnldlwvtvyygvpvwkeakttlfcasdakaydkevhnvwathacvptdpnpqeivlenvten
fnmwkndmvdqmhediislwdqslkpcvkltplcvtlncknvnisananatatlnssmngeikncsfntttelrdkkqkvyalfyk
pdvvplnggehnetgeyilincnsstitqacpkvsfdpipihycapagyailkcnnktfngtgpcnnvstvqcthgikpvvstqll
lngslaeeeiivrsenltnniktiivhlnksveikctrpnnntrksvrigpgqtfyatgeiigdireahcnisretwnstliqvke
klrehynktikfepssggdlevtthsfncrgeffycdttklfnetklfneseyvdnktiilpcrikqiinmwqevgramyappieg
nitcksnitgllltwdggenstegvfrpgggnmkdnwrselykykvveikplgvaptkskrkvvgrekravglgavllgflgaags
tmgaasitltvqarqllsgivqqqsnllraieaqqhllqltvwigikqlqtrvlaierylkqqllglwgcsgklicatavpwnssw
snkslgdiwdnmtwmqwdreisnytntifrlledsqnqqeknekdllaldswknlwnwfditnwlwyikifimivggliglriifg
vlaivkrvrqgysplsfqtlipnprgpdrlgrieeeggeqdkdrsirlvsgflalawddlrslclfsyhqlrdfiltaaraaellg
rsslrglqrgwevlkylgnlvqywglelkrsainlfdtiaiavaegtdriieviqricrairyiptrirqgfeaall
CD5-25711 pg160
(SEQ ID NO: 15)
MPMGSLQPLATLYLLGMLVASVLAnlwvtvyygvpvwkeakttlfcasdakgydkevhnvwathacvptdpnpqemplenvtenfn
mwendmvnqmhedvislwdeslkpcvkltplcvtlnctdvnknvsssdtdnyketmkerknctfnmttelrdknqkkyalfykldi
vplddndnasyrlincntstltqacpkvsfdpipihycapagyailkcknktfngigpcnkvstvqcthgikpvvstqlllngsla
eedivirsenitdnaktiivhlnesveivcirpnnntrksirigpgqtfyatgdivgdirqaycnisegkwnktlqrvseklaehf
pnstinfnsssggdleitthsfncggeffycntsglfngtymnndtksndtksnsssiitipcrikqiinmwqevgravyappiag
nitcksnitgilltrdggrgeevkndtetfrpgggnmkdnwrselykykvveikplgvaptaakrrvverekravglgavllgflg
aagstmgaasitltvqarqllsgivqqqsnllraieaqqhmlqltvwgikqlqarvlaierylkdqqllgiwgcsgklicttavpw
nsswsnknqteiwdkmtwmqwdreisnytdtiyrlledsqnqqeknekdlleldkwqnlwswfnitnwlwyirifimivggliglr
iifavlsivnrvrqgysplsfqtlapnprgldnlgrieeeggkedrnrsirlvhgflalawddlrslclfsyhrlrdlilliarav
ellgqrgwealkylagivqywglelkksavslfdtiaiavaertdriigliqgicraicniprrirqgfeaalq
CD5-Q769.d22 pg160
(SEQ ID NO: 16)
MPMGSLQPLATLYLLGMLVASVLAagnlwvtvyygvpvwrdaettlfcasdakaydreahnvwathacvptdpspqevplgnvtee
fnmwknnmveqmhtdiislwdqslqpcvkltplcvtlncsnsnnipsvsnitddmkeeikncsfnmttelkdkkqnvyslfyrldv
vpletktnqnsshsryrlincntsaitqacpkvsfepipihycapagfailkcndkgfngtglcknvstvqcthgikpvvstqlll
ngslaegkvmvrsenitnnakniiiqfnnsvqinctrpgnntrksihlgpgkvfyatdiigdirkahcnvnrqqwnktlqdvatql
rthfrnrtiifnnslggdleitthsfncrgeffycntsglfngiwngtqepnrtesndtitlqcrikqiinmwqrvgqaiyappiq
geircesnitgliltrdggiinsteetfrpgggdmrdnwrselykykvvkieplgvaptkakrrvverekravgfgafflgfigaa
gstmgaasitltvqarqllsgivqqqnnllraieaqqhllkltvwgikqlqarvlaverylkdqqllgiwgcsgkfictttvpwns
swsnksqseiwdnmtwmqwdkeinnytqiiydlieesqrqqekneqdllaldkwanlwnwfdisnwlwyikifimivggliglria
favlsvinrvrqgysplsfqthtpnprdldrpgrieeeggeqdrdrsirlvsgflalawddlrslclfsyhrlrdfilvaartvel
lghislkglrrgweglkylgnllsywgrelkisainlldtiaivvaewtdriieigqrlcraiiniprrirqgferall
CD5-Q842.d12 gp160
(SEQ ID NO: 17)
MPMGSLQPLATLYLLGMLVASVLAvdnlwvtvyygvpvwkeaettlfcasdakayetekhnvwathacvptdpnpqeihlenvtee
fnmwknnmveqmhtdiislwdqslkpcvkltplcvtldcnnvtnngtsdmreeikncsfnmttelrdkrqkvyslfykldivqine
dqgnssnnkyrlitcntsaitqacpkvtfepipihycapagfailkckdeefngigpcknvstvqcthgikpvvstqlllngslae
kevkircenitnnaktiivqlvnpvkinctrpnnntrksihigpgqafyatgdiigdirqahcnvnrtewnntlhqvveqlrkhfn
ktinfanstggdleitthsfncggeffycnttnlfnstwnhtasmnstesndtiilpcrikqiinmwqrvgqamyappirgvirce
snitgliltrdggntnstretfrpgggdmrdnwrselykykvvkieplgvaptkakrrvverekravgigavfigflgaagstmga
asitltvqarqllsgivqqqsnllraieaqqhllkltvwgikqlqarvlaverylkdqqllgiwgcsgklicttsvpwnsswsnks
qneiwdnmtwlqwdkeisnytqiiydlleesqnqqekneqdllaldkwanlwnwfdisnwlwyikifimivggliglrivfavlsv
inrvrqgysplsfqthtpnprgldrperieeeggeqdknrsirlvsgflalawddlrslclfsyhrlrdfilivartvellghssl
kglrlgweglkylgnllsywgrelrisatnlldtiaiviagwtdrvieigqrlcraflniprrirqgferall

Q. Statistical Reporting

All measurements were from distinct samples. Replicate deep mutational scans were performed independently on different transfected cultures. Central tendency, variation, sample sizes, and statistical tests are described in figure legends.

R. Data Availability
Analyzed (as an Excel spreadsheet) and raw deep sequencing data are deposited in NCBI's Gene Expression Omnibus⁷⁸ under series accession number GSE102276. This includes commands for running Enrich scripts to replicate data analysis.

S. Plasmid Availability

Plasmids have been deposited with Addgene: www.addgene.org/Erik_Procko/

### Example 2. Deep Mutational Scanning of HIV-1_{BaL} Env

Codon-optimized Env of the BaL isolate (a tier 1B virus from clade B)⁴⁵ with a CD5 leader sequence for enhanced surface expression⁴⁶ bound soluble CD4 (domains D1-D2), PG16 and VRC01 by flow cytometry when expressed on human Expi293F cells. Previously, deep mutational scanning of HIV-1 receptors CCR5 and CXCR4 found only qualitative agreement between replicate experiments⁴⁷, likely in part due to under sampling the diverse mutant libraries. To increase sampling of mutations in the much larger Env protein, three separate single site-saturation mutagenesis (SSM) libraries were constructed focused on the Env N-terminus (a.a. 31-265; numbering based on the HXB2 reference strain), center (a.a. 266-529), and C-terminus (a.a. 530-856). Combined data from separate sorting experiments of each SSM library encompass 16,332 of the possible 16,520 single amino acid substitutions.

To maintain a tight link between genotype and phenotype, the Env libraries were transfected into Expi293F cells under conditions that yielded close to one sequence variant per cell, achieved by diluting the plasmid-based libraries with a large excess of carrier DNA⁴⁷. However, when diluted to a single sequence variant per cell. Env expression was barely detected. It was critical that expression be increased by addition of an artificial intron in the 5′ untranslated region, and cotransfection with carrier DNA hypothesized to promote extra-chromosomal replication of the episomal Env plasmids.

Expi293F cells expressing the Env SSM libraries were bound to soluble CD4, VRC01, or PG16 near the apparent dissociation constants, and were screened by fluorescence-activated cell sorting (FACS) for highest binding signal (Table 1).

TABLE 1
Sorting Conditions for Env_{BaL} Deep Mutational Scanning.

| | | Collected Cells | |
|---|---|---|---|
| Ligand | Gating | Replicate 1 | Replicate 2 |
| sCD4(D1D2) | Top 0.5% Library A¹ | 210,000 | 259,000 |
| (200 nM) | Top 0.5% Library B | 233,000 | 101,000 |
| | Top 1.0% Library C² | 178,000 | 486,000 |
| VRC01 | Top 0.5% Library A | 95,000 | 162,000 |
| (5 nM) | Top 0.5% Library B | 90,000 | 155,000 |
| | Top 1.0% Library C | 180,000 | 203,000 |
| PG16 | Top 0.6% Library A | 79,000 | 92,000 |
| (2 nM) | Top 0.6% Library B | 217,000 | 222,000 |
| | Top 0.9% Library C | 136,000 | 183,000 |

¹Library A spans a.a. 31-265, Library B a.a. 266-529; Library C a.a. 530-856.
²Of the three SSM libraries, Library C had the most positive cells after ligand staining, as there are few deleterious imitations in the cytosolic Env tail. Therefore to sort cells with similar binding signals, a higher percentage of Library C was gated and collected.

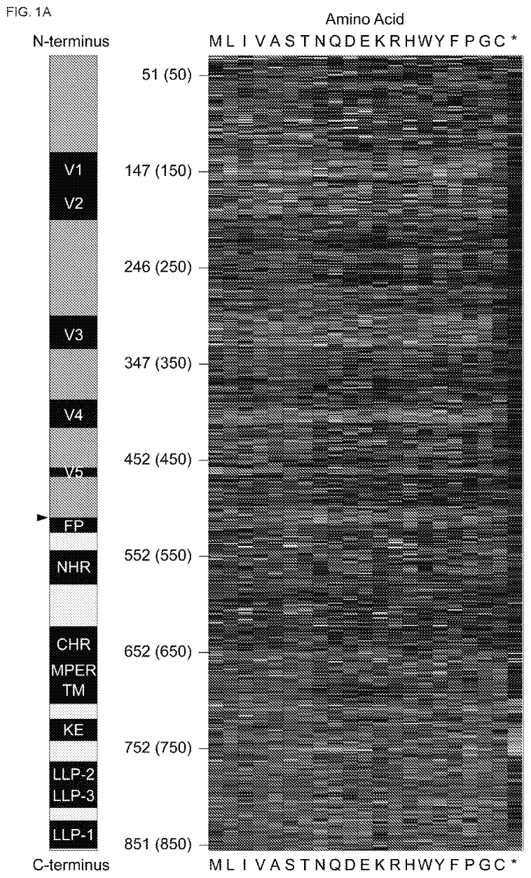

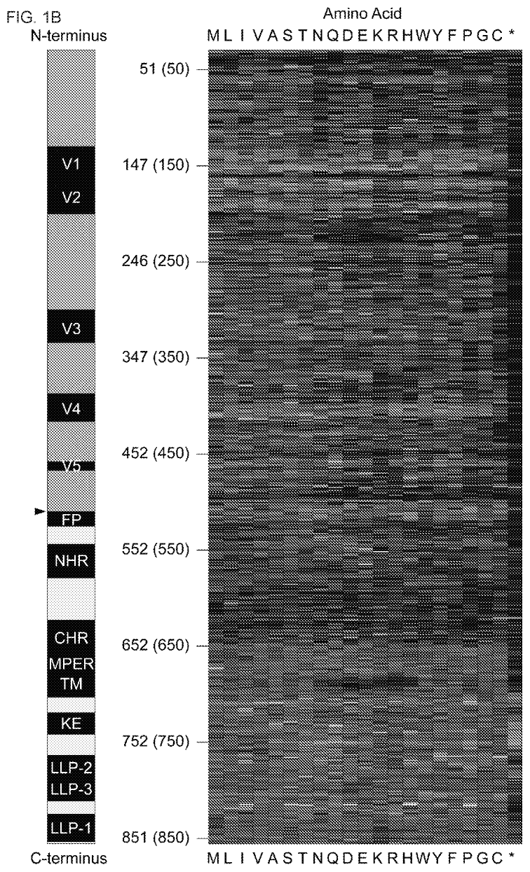

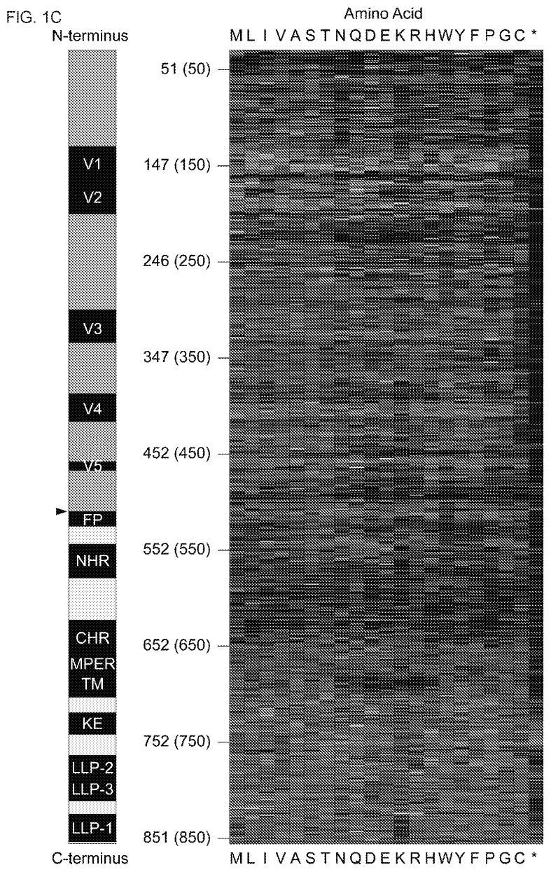

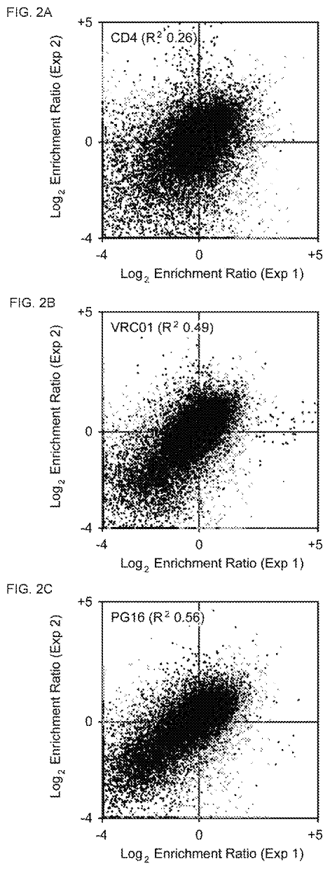

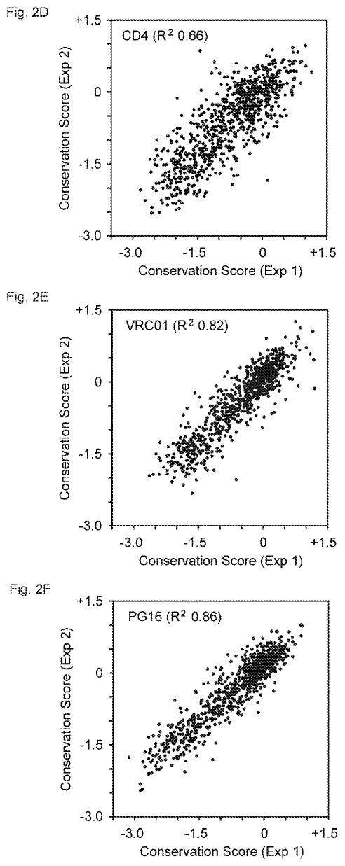

The enrichment or depletion of Env mutants was determined by comparing the frequencies in the näive plasmid libraries with the transcripts in the evolved populations (FIG. 1). Enrichment ratios for each amino acid substitution qualitatively agreed between replicate experiments (FIG. 2A-C). Env evolution experiments for CD4 binding had the lowest agreement, perhaps due to poor reproducibility when sorting on a very weak fluorescent signal compared to the antibody binding screens. Conservation scores (mean of the log₂ enrichment ratios for all substitutions at a specific position) were closely correlated between replicate experiments (FIG. 2D-F). We consequently identify functional sites with tight sequence constraints from the conservation scores, and validate single mutations of interest individually.

### Example 3. Env Sequence-Activity Landscapes for Interacting with CD4, VRC01 and PG16

The Env sequence-activity landscapes are similar whether screened for CD4, VRC01 or PG16 binding (FIG. 1); this is because features of the landscapes that impact protein folding and surface expression will be shared. The highest conservation is in regions maintaining non-covalent association between gp41 and gp120 subunits. This includes gp41 residues both within and upstream of the C-terminal heptad repeat that coil around the similarly conserved gp120 N- and C-termini. The N-terminal sequence prior to the first variable loop (V1) that forms the gp120 inner domain is also highly conserved, and polar substitutions within the hydrophobic transmembrane (TM) helix are depleted. By comparison, V1 to V5 tolerate many substitutions, as do residues on both sides of the furin proteolysis site and within the gp41 fusion peptide.

Premature stop codons prior to the membrane-spanning helix (a.a. 684-705) are depleted, as expected. However, stop codons are tolerated in the cytosolic C-terminus immediately following the membrane anchor; this region contains a GYSPL motif that interacts with the AP-2 complex for clathrin-mediated endocytosis, normally maintaining low levels of surface Env expression^{48,49}. Premature stop codons are again depleted around the N-terminus of the Kennedy Epitope, but then become highly enriched around residues 731-759. Env C-terminal deletions have previously been shown to increase surface expression⁵⁰, which has been appropriated for elevated Env levels in virus-like particle vaccines. This indicates that even higher Env surface expression might be achieved in virus-like particles by using alternative premature stop codons (e.g. at position 731) to what have already been tested (for example^{51,52}).

Stop codons are again depleted at a.a. 760-782 and 795-837, approximately corresponding to the lentivirus lytic peptide-2 (LLP-2) and LLP-3 to LLP-1 regions, respectively. Premature stop codons are weakly enriched near the very C-terminus, removing another endocytosis signal at the very end of the protein⁵³ that would otherwise reduce the surface expressed pool. Lysine substitutions are depleted around a.a. 832-851 in LLP-1, in agreement with prior observations that Env expression is decreased when lysine is mutated into the arginine-rich LLP-1 region⁵⁴, despite arginine and lysine sharing similar physicochemical properties. Overall, there are clearly distinct cytosolic sequence elements that modulate surface expression, and yet there is little sequence conservation after the TM helix, suggesting regulatory cytosolic elements lack folded structure. Disordered cytosolic tails with embedded regulatory motifs are common amongst transmembrane proteins⁵⁵.

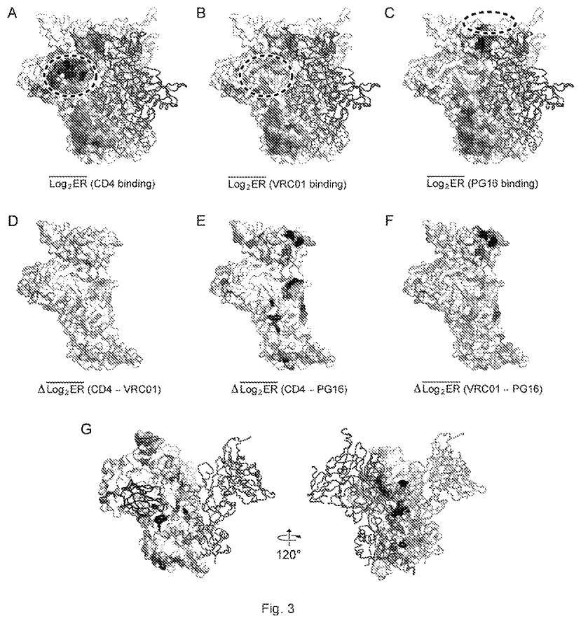

While the Env sequence-activity landscapes for interacting with the three protein ligands are similar when viewed globally, unique surface features are immediately apparent when the conservation scores are plotted on a model of trimeric BaL Env in the closed conformation (FIG. 3A-C). In particular, different surface patches of gp120 are uniquely conserved for interacting with CD4, VRC01 or PG16, while the surface where gp41-gp120 associate is conserved for all three interactions, likely for correct protein folding and surface presentation. To highlight the regions explicitly conserved for interacting with different ligands, difference plots were mapped to the structure, in which conservation scores for interacting with one ligand are subtracted from those for interacting with a second (FIG. 3D-G). The most notable differences are localized to the structurally characterized VRC01 and PG16 epitopes, the known CD4 binding site, and trimer interfaces.

### Example 4. Env Residues within the CD4 Binding Site are Conserved

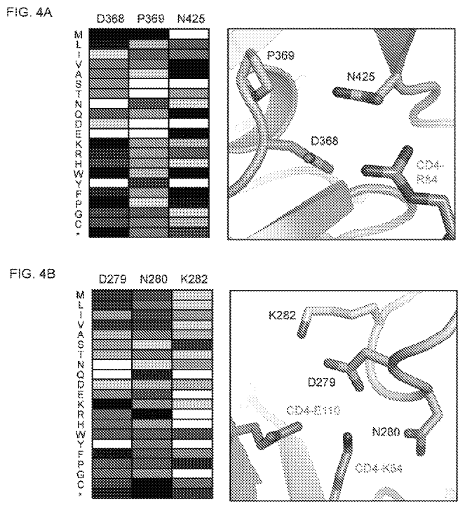

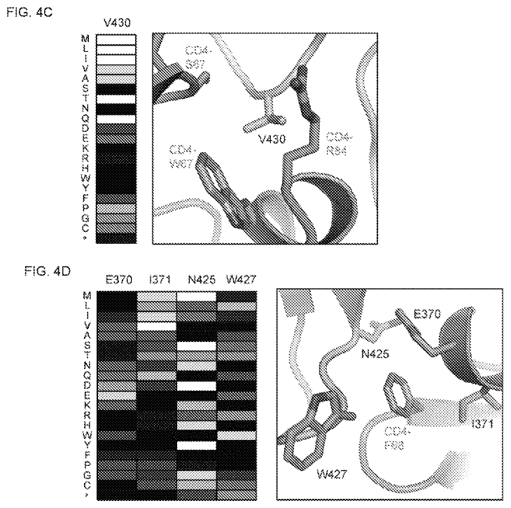

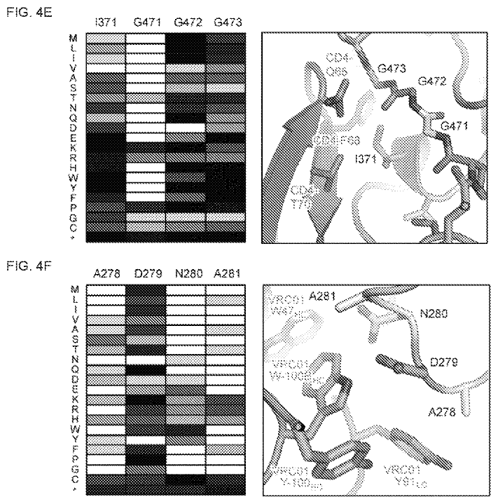

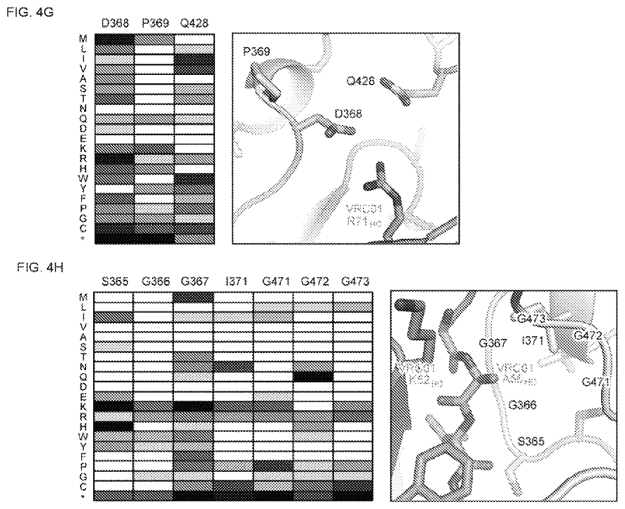

CD4 binds the gp120 outer domain, stabilizing structural elements in the conformationally flexible subunit^{13,56,57}. The defined binding site is highly conserved in the selection for CD4 binding (FIGS. 3A, 3D-E and 3G), and mutation phenotypes agree with atomic modeling (FIG. 4A-E). For example, Env-V430 packs within a hydrophobic pocket formed by CD4-W87 and the aliphatic chain of CD4-R84, and Env-V430 substitutions to small or aliphatic hydrophobic residues are tolerated for CD4 binding (FIG. 4C). CD4-F68 is sandwiched between highly conserved Env-W427 and Env-I371, which is primarily restricted to aliphatic hydrophobic side chains M, L, I and V (FIG. 4D). The guanidinium group of CD4-R84 contacts or is in close proximity to Env-D368, P369, and N425; most acidic substitutions of these three Env residues are enriched (FIG. 4A). Finally, Env-G472 and G473 lie flush against a β-sheet surface of CD4 and are highly conserved, whereas the α-carbon of neighboring Env-G471 is directed to a cavity and tolerates most substitutions (FIG. 4E).

### Example 5. VRC01-Env Association Tolerates Env Sequence Diversity

VRC01 engages the CD4-binding site on the gp120 outer domain⁵⁸, and yet this surface is distinctively not conserved in the Env sequence-activity landscape for VRC01 interaction (FIGS. 3B, 3D and 3F). Rather, VRC01 binding is resistant to most Env single amino acid substitutions within its structurally characterized interface, a result that was suggested by previous small-scale mutational analysis⁴². This is an ideal property of a broadly neutralizing antibody, which limits mechanisms for viral escape. To illustrate with just one example, aromatic antibody side chains within the interface core engage Env loop residues 278-281; only Env-D279 in the loop, which contacts the indole NH of VRC01-W100B_{HC} (Kabat numbering), is moderately conserved in the selection (FIG. 4F-H). Aromatic side chains with hydrogen-bonding potential may be ideally suited to interacting with diverse antigen targets⁵⁹.

### Example 6. Residues at Env Subunit Interfaces are Under Selection for PG16 Binding

PG16 binds the junction between two gp120 subunits at the Env apex, possibly forming bridging contacts to glycans from each subunit and explaining the antibody's strong preference for trimeric quatemary structure⁴⁰. Crystal structures of PG16 and related bNAb PG9 bound to scaffolded V1-V2 demonstrated extensive contacts to the glycan on Env-N160, with a smaller contact surface to the adjacent glycan on Env-N156 (alternatively N173 in other HIV-1 strains)^{31,60}. This correlates with mutagenesis data demonstrating greater importance of the N160 glycosylation motif³⁹. An unusually long PG16 HCDR3 penetrates between the glycans and forms sequence-independent n-sheet-like interactions with the V1-V2 backbone^{31,60}. Consistent with these prior results, most mutations of Env residues within the epitope are tolerated in the deep mutational scan for PG16 binding, with the critical exception of the conserved N160 glycosylation motif (FIGS. 4I, 4J).

When the Env SSM library is evolved for PG16 interactions, trimer interface residues are under selection and show moderate sequence conservation (FIGS. 3C, and 3E-G). This is most apparent in the difference plot between conservation scores for CD4 and PG16 binding (FIGS. 3E and 3G), which preferentially bind monomeric gp120 and trimeric Env, respectively. Therefore, PG16 binding could be used as a selection for evolving Env variants stabilized in a closed, trimeric conformation. However, many previously characterized mutations for stabilizing closed SOSIP trimers were notably neutral in the Env_{BaL}-PG16 sequence-activity landscape, including E64K¹⁷, H66R¹⁷, A73E²², S110E²², A316W¹⁷, (Q/R/K)432P²³, and I559P¹⁹, with only A433P²³ being slightly enriched (however, A73E, S110E and A433P are depleted following the selections for CD4 binding). These mutations were identified in SOSIP constructs (primarily from the clade A BG505 strain) using a variety of strategies, including favorable antigenic profiles, trimer stability, purification, structure, and reduced exposure of CD4i epitopes. Efforts to identify alternative conformation-stabilizing mutations based on Env_{BaL}-PG16 binding complements these earlier studies, expanding conformational engineering to full-length Env from a broader range of HIV-1 strains.

Nearly a hundred mutations in the Env sequence-activity landscape for PG16 binding had log₂ enrichment ratios greater than 1.5 in both selection replicates, though it is noted that because most substitutions in the N-terminal SSM library were severely depleted, neutral substitutions in V1-V2 could be misleadingly enriched. 42 substitutions that were highly enriched in both replicate selection experiments (Table 2) were individually tested. The screen was biased towards residues located on subunit surfaces in areas less likely to interact with bNAbs, including trimer or gp120-gp41 interfaces, and around the furin cleavage site.

TABLE 2
Env_{BaL} mutants and PG16 binding.

| Env Variant (HXB2 numbering) | PG16 binding¹ |
|---|---|
| Wildtype | +++² |
| L34Y | +++ |
| W35Y | +++ |
| T49D | ++++ |
| T51Q | +++ |
| Y61K | +++ |
| Y61Q | +++ |
| E106S | + |
| Q114A | ++++ |
| K117V | +++++ |
| K117Y | +++++ |
| P124D | ++++++ |
| I161L | ++ |
| T163D | ++++ |
| N164P | ++ |
| I165H | ++ |
| I165L | ++ |
| I165Q | +++ |
| R166E | +++++ |
| R166F | ++++ |
| R166L | +++++ |
| V200E | +++++ |
| V200T | ++++ |
| F223Y | +++++ |
| T244I | ++ |
| R315A | ++++++ |
| R315Q | ++++++ |
| V430E | +++ |
| R432T | +++++ |
| K500Q | +++ |
| A501E | ++ |
| E509Q | +++ |
| G514P | ++++ |
| G516Q | ++++ |
| R557Q | ++++ |
| H564A | +++ |
| L568Y | +++ |
| L581D | ++++ |
| I595M | ++++ |
| Q658F | +++ |
| E662Q | +++ |
| L663N | ++++ |
| K665N | +++ |
| S700Q | +++ |
| V200E + F223Y | +++++ |
| R432T + R557Q | ++++ |
| R315A + L663N | ++++++ |
| T49D + I595M | +++ |
| R166L + L581D | +++ |
| G514P + G516Q | +++ |
| Q114A + V200T | ++++ |
| K117V + T163D | ++++ |
| K117V + R315A | ++++++ |
| Q114A + L663N | ++++ |
| V200T + I595M | ++++ |
| T49D + R315A + I595M | ++++++ |
| R166E + F223Y + L663N | ++++ |
| K117V + R166L + R315A | +++++ |
| R166E + R557Q + L581D | +++ |
| K117V + R166L + F223Y | ++++ |
| T163D + V200T + L581D | ++++ |
| R166L + R315A + G514P | +++++ |
| R315A + L663N + T49D | +++++++ |
| R315A + L663N + R166L | +++++ |
| R315A + L663N + F223Y | ++++++ |
| R315A + L663N + R432T | +++++ |
| R315A + L663N + I595M | ++++++ |
| T49D + P124D + I595M (BaL-QES.i01) | ++++++++ |
| P124D + L663N | ++++++ |
| T49D + R315A + I595M + K117Y | ++++++ |
| T49D + R315A + I595M + R166L | ++++++ |
| T49D + R315A + I595M + L663N | +++++++ |
| T49D + K117V + R315A | +++++++ |
| K117V + R315A + L663N | ++++++ |
| K117V + R166L + F223Y + I595M | +++++ |
| K117V + R166L + F223Y + L663N | +++++ |
| P124D + R315A | ++++++ |
| P124D + R315A + L663N | ++++++ |
| T49D + R315A + I595M + L663N | ++++++ |
| T49D + P124D + R315A + I595M + | ++++++ |
| L663N | |
| T49D + P124D + R315A + I595M | +++++++ |
| T49D + P124D + I595M + L663N | +++++++ |
| T49D + P124D + R315A + G514P + | ++++++ |
| I595M | |
| T49D + P124D + L663N (BaL-QES.i02) | +++++++ |

¹Expi293F cells expressing Env mutants were stained with 0.5 and 2 nM PG16 (n ≥ 2), and binding was assessed by flow cytometry.
²+ is below 0.75 wildtype signal, ++ is 0.75-0.9x, +++ is 0.9-1.1, ++++ is 1.1-1.25x, +++++ is 1.25-1.5x, ++++++ is 1.5-1.75x, +++++++ is 1.75-2x, and ++++++++ is > 2x wildtype binding signal.

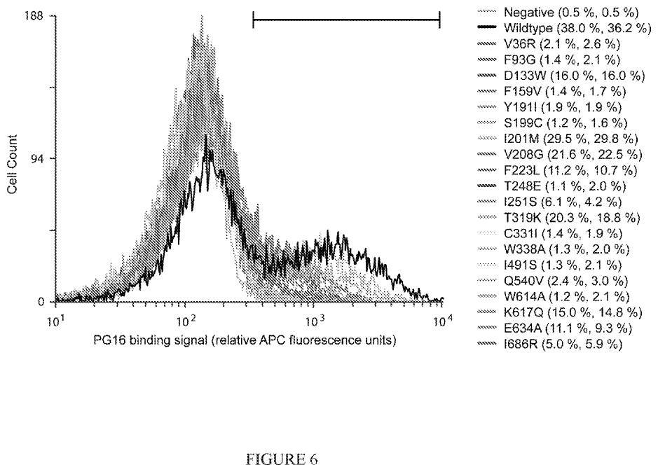

Twenty mutations were validated to cause a slight to moderate increase in PG16 binding, and these are clustered at five sites (FIG. 5). We refer to these as Quatemary Epitope Selected (QES) mutations. It is noteworthy that finding gain-of-function mutations is challenging, and presuming such mutations are uncommon, one expects them to be difficult to find amongst the noise of many neutral or deleterious substitutions in a mutational scan. To emphasize this and provide context to the result that 20 out of the 42 tested mutations successfully increased PG16 binding, we also tested by targeted mutagenesis 20 representative depleted mutations from the selection experiments, and found all 20 were indeed deleterious for PG16 binding (FIG. 6).

The first site of Env mutations that enhance PG16 binding is located at the trimerization interface near the apex (FIG. 5). Twelve of the 20 mutations are found here: Q114A, K117V/Y, P124D, T163D, R166E/F/L, V200E/T, R315A, and R432T. Nearly all of these mutations reduce positive charge at the apical trimer interface, either through substitution of a basic residue, or introduction of an acidic residue (or both). Furthermore, most substitutions of Env-K117, R166, and R432 are predicted to enhance PG16 binding in the sequence-activity landscapes. This unambiguously highlights that neutralization of the electropositive apical trimer interface stabilizes a conformation with increased PG16 binding signal. In this atomic model of BaL Env, this positive region extends 30 Å from the apical surface down the central cavity along the C₃ axis. It is likely that this electropositive region imposes a ‘spring-loaded’ mechanism to Env opening, with electrostatic repulsion between apical tips priming the subunits for conformational changes upon receptor binding.

The second site of Env mutations for enhanced PG16 binding is a centrally located interfacial region where residues are in contact between Env protomers, and between gp41 and gp120 subunits (FIG. 5). F223Y in one Env protomer may add a hydrogen bond contact to R557 or N553 from a neighboring protomer. Likewise, T49D would add a salt-bridge contact to R557 of the neighboring protomer. R557Q reduces the desolvation penalty of burying a charged group at the interface, yet may still hydrogen bond to partners across the trimer interface. Finally L581D would add favorable electrostatic interactions with R557 and R579 on a neighboring promoter.

The mutation I595M is found in site 3 at a trimer contact (FIG. 5). I595 of one gp41 subunit occupies a hydrophobic pocket on an adjacent gp41; the methionine substitution may better pack in this pocket. Site 4 is proximal to the furin cleavage site (FIG. 5), and includes two mutations (G514P and G516Q) that weakly increase PG16 binding for unknown reasons. Finally, mutation L663N in site 5 (FIG. 5) has an unknown structural effect; residue 663 begins the membrane-proximal external region (MPER) and the structure here is poorly characterized.

### Example 7. Stabilization of an Env Conformation for Enhanced PG16 Binding

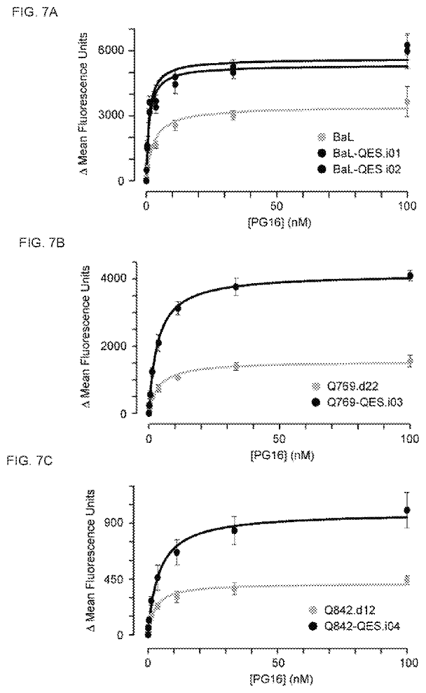

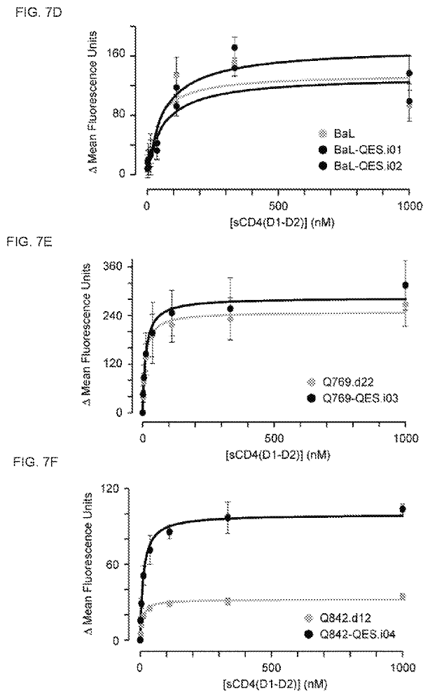

Mutations to engineer BaL Env for enhanced PG16 binding (Table 2) were combined. Multiple Env variants with similarly high PG16 binding signals were identified, often combining mutations from separate sites (mutations within a single site likely have epistatic interactions that limit their benefit when combined). Two sequences, termed BaL-QES.i01 and BaL-QES.i02 (“i” for “interfaces”, where most of these mutations are localized), were chosen for further characterization. These variants combine mutations T49D; P124D; I595M and T49D; P124D; L663N, respectively. The CD4 binding potential of BaL-QES.i01 and BaL-QES.i02 is unchanged from wildtype (FIG. 7D), but the proteins display a 1.6-fold increase in PG16 binding at saturation (FIG. 7A), with slightly tighter affinity (apparent K_{D} values for PG16 affinity are 4±1 nM for wildtype, 1.2±0.4 nM for BaL-QES.i01, and 1.4±0.5 nM for BaL-QES.i02). This data is consistent with expression of diverse conformations on the cell surface, with stabilizing mutations increasing the pool of Env occupying a closed, trimeric state competent for PG16 interaction.

Env loses conformational flexibility upon proteolytic processing, increasing affinity for certain conformation-dependent antibodies, including PG16⁶². Western blot shows that wildtype, QES.i01 and QES.i02 gp160_{BaL} were all cleaved to gp120 (FIG. 8A), and over-expression of furin to mitigate any differences in furin-dependent cleavage did not change the central observation that more PG16 binds to cells expressing QES mutants (FIG. 8B).

### Example 8. QES Mutations are Partially Transferable to Other Env Strains

Most of the QES mutation sites identified in BaL Env are conserved across HIV-1 strains and clades, though in some cases other strains already carry the substituted amino acid, such as a threonine or tyrosine at positions 200 and 223. (Based on this observation, R315 of gp160_{BaL} was substituted for glutamine, which is found at the equivalent position of Env in other HIV-1 strains, and again found PG16-binding was increased; Table 2.) A subset of the mutations in BaL Env that enhance PG16 binding was tested to determine if the mutations are effective in other HIV-1 strains. Q769.d22 and Q842.d12 are tier 2 strains from clade A, YU-2 is a tier 2 strain from clade B, and 25711 and DU422 are tier 1B and 2 strains from clade C, respectively⁴⁵.

The mutations generally had little positive impact on PG16 binding to YU-2, 25711, or DU422 Env. However, many of the QES mutations increased PG16 binding to Q769.d22 and Q842.d22 Env (Table 3), and combinations of mutations were screened for even higher PG16 binding. Q769-QES.i03 has three mutations (A200E; F223Y; I595M), and bound PG16 with similar affinity (apparent K_{D} of 3.7±0.6 nM and 5±3 nM for Q769-QES.i03 and wildtype Env, respectively) but 2.7-fold higher binding at saturation (FIG. 7B).

TABLE 3
PG16 binding to subunit interface mutants of Env from different
HIV-1 strains

| YU2 Variant (HXB2 numbering) | 1.5 nM PG16¹ |
|---|---|
| Wildtype | +++² |
| T49D | +++ |
| K117Y | ++ |
| P124D | ++ |
| T163D | + |
| R166L | ++ |
| V200E | +++ |
| F223Y | +++ |
| R315A | ++ |
| K432T | +++ |
| R557Q | +++ |
| L581D | ++ |
| I595M | +++ |
| L663N | +++ |
| | 1.5 nM PG16 |
| 25711 Variant | |
| Wildtype | +++ |
| K49D | + |
| E114A | ++ |
| K117Y | + |
| P124D | + |
| T163D | + |
| R166L | + |
| T200E | + |
| R432T | +++ |
| R557Q | ++ |
| L581D | ++ |
| I595M | +++ |
| L663N | +++ |
| DU422 Variant | |
| Wildtype | +++ |
| K49D | + |
| Q114A | ++++ |
| K117Y | ++ |
| P124D | ++ |
| T163D | + |
| R166L | +++ |
| T200E | ++ |
| R310A | +++ |
| R432T | +++ |
| R557Q | +++ |
| L581D | ++ |
| I595M | +++ |
| L663N | +++ |
| Q769.d22 Variant | |
| Wildlype | +++ |
| Q114A | +++ |
| P124D | ++++++ |
| T163D | + |
| K166L | +++ |
| A200E | ++++++ |
| F223Y | ++++ |
| K315A | +++++ |
| R557Q | ++++ |
| L581D | +++ |
| I595M | ++++ |
| L663N | ++++ |
| P124D + A200E | +++ |
| P124D + I595M | +++++++ |
| P124D + F223Y + I595M | ++++++++ |
| A200E + F223Y + I595M (Q769-QES.i03) | +++++++++ |
| K315A + K166L | ++ |
| P124D + R557Q | +++++++ |
| A200E + K166L | ++ |
| R557Q + F223Y | +++++ |
| P124D + A200E + F223Y + I595M | ++++ |
| A200E + F223Y + R557Q + I595M | +++++++ |
| Q842.d12 Variant | 30 nM P616 |
| Wildtype | +++ |
| Q114A | +++ |
| K117Y | +++ |
| P124D | ++++ |
| T163D | +++ |
| R166L | ++++ |
| A200E | ++++ |
| F223Y | +++ |
| R557Q | ++++ |
| L581D | +++ |
| I595M | +++++ |
| L663N | +++ |
| R166L + A200E | ++++ |
| R166L + R557Q | +++++ |
| R166L + R557Q + I595M | ++++ |
| A200E + R557Q + I595M | +++++ |
| P124D + A200E | +++++ |
| P124D + R166L | +++++ |
| P124D + R557Q + I595M | ++++++ |
| P124D + R166L + R557Q | ++++ |
| R166L + A200E + R557Q | ++++ |
| R166L + F223Y + R557Q | +++++ |
| P124D + R166L + R557Q + I595M | +++++ |
| P124D + F223Y + R557Q + I595M (Q842-QES.i04) | +++++++ |

¹Assessed by flow cytometry analysis of transfected Expi293F cells. n = 3, except for 25711 where n = 2.
²+ is below 0.75 wildtype signal, ++ is 0.75-0.9x, +++ is 0.9-1.1, ++++ is 1.1-1.25x, +++++ is 1.25-1.5x, ++++++ is 1.5-1.75x, +++++++ is 1.75-2x, and ++++++++ is >2x wildtype binding signal.

Q769-QES.i03 and wildtype Q769.d22 Env bound equivalent levels of soluble CD4 (FIG. 7E). Q842-QES.i04 has four mutations (P124D; F223Y; R557Q; I595M), and again bound PG16 with similar affinity (apparent K_{D} of 5±2 nM and 3.4±0.6 nM for Q842-QES.04 and wildtype Env, respectively). Q842-QES.i04 Env displayed greatly increased PG16 binding signal at saturation (2.4-fold; FIG. 7C), though this may be explained by an increase in surface expression based on a similar 3-fold increase in soluble CD4 binding (FIG. 7F). As observed for BaL Env, the variants with the highest PG16 binding signals generally combined mutations from different sites.

Neutralization of positive charge at the BaL, Q769.d22 and Q842.d12 Env apex increased presentation of the PG16 epitope, and excess positive charge within the turret may prime the closed Env trimer for opening by simple electrostatic repulsion. Instability of closed Env due to positive charge at the apex was also proposed in a recent structural study of PGT145-bound BG505 SOSIP, and it was further hypothesized that an arginine at position 315 increases the dynamics of apex opening⁶¹. However, no correlation between the identity of residue 315 and which Env sequences can be stabilized by neutralizing apex mutations was identified.

### Example 9. QES Mutants are Competent for Catalyzing Membrane Fusion

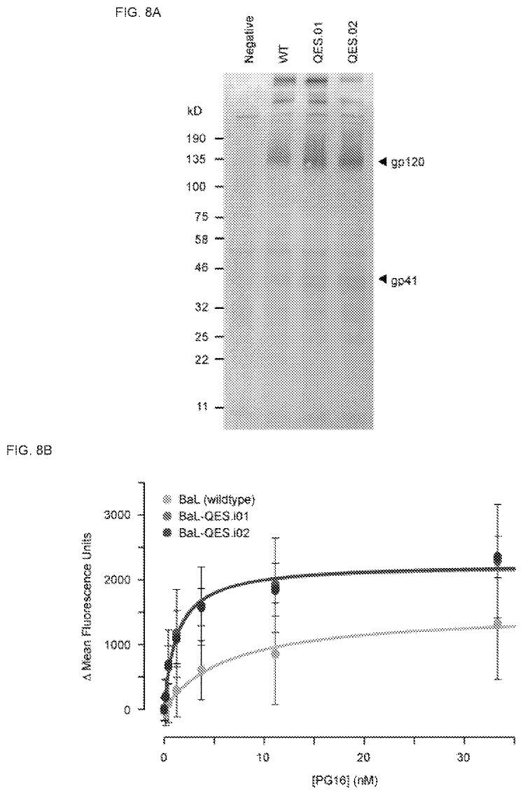

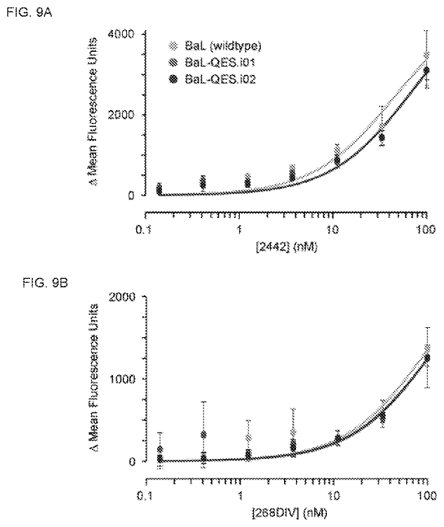

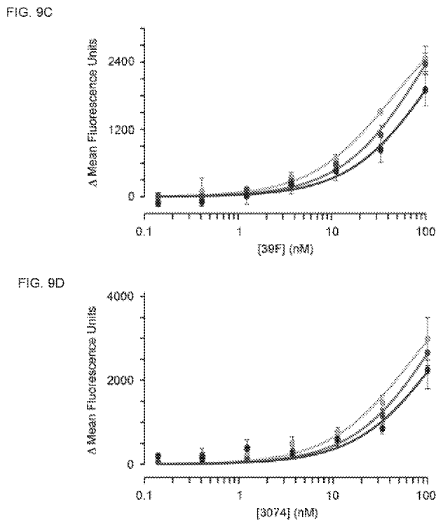

The QES mutations described thus far stabilize the closed conformation recognized by PG16 but do not diminish CD4 binding, and BaL-QES.i01 and QES.i02 variants still bind V3 region antibody clones 2442, 268-D IV, 39F, and 3074 similarly to wildtype Env (FIG. 9). The variants therefore still sample or can be induced into the open conformation for V3 antibody recognition and tight CD4 binding. It therefore seemed likely that the QES variants might catalyze membrane fusion similar to wildtype Env. Using flow cytometry to detect enlarged syncytia when Env-expressing and CD4/CCR5-expressing cells are co-incubated, BaL-QES.01 and BaL-QES.02 Env were found to mediate membrane fusion at levels similar to or even greater than the wildtype protein (FIGS. 10A and 10B). This is despite the possibility that QES mutations may impair later steps in the fusion pathway (for example, it is predicted L663N will disrupt the gp41 post-fusion state). This is consistent with the closed state of Env being a relevant conformation for target cell interaction and infection.

### Example 10. A QES Mutation within the Core of Env can Destabilize the CD4-Bound State

From the deep mutational scan, mutations within the Env core to enhance PG16 binding were investigated. Mutations to 12 buried residues were incorporated into a combinatorial library of extracellular gp140_{BaL} fused to a non-native TM helix for surface display; this served to both increase surface expression for higher PG16 binding signals, and ensured the selection didn't simply enrich for premature stop codons in the cytoplasmic tail. The combinatorial library was sorted for binding to PG16 for three rounds, and then individual clones were screened. An alignment of 7 sequences with elevated PG16 binding highlighted mutations I181L, T202N, V254T and V255M (FIG. 11A). Further characterization of one sequence, clone-27, revealed that while PG16 binding to extracellular gp140 was increased (FIG. 11B), this set of mutations instead diminished PG16 binding to gp160 (FIG. 11C), and therefore extracellular gp140 is an imperfect replacement for studying full Env. The four consensus mutations were therefore tested individually in full-length Env; I181L, V254T, and V255M increased PG16 binding, while T202N was deleterious and excluded (Table 4).

TABLE 4
PG16 binding to core mutants of Env from different HIV-1 strains

| BaL Variant (HXB2 numbering) | 1.5 nM PG16¹ |
|---|---|
| Wildtype | +++ |
| I181L | ++++ |
| T202N | + |
| V254T | ++++ |
| V255M | ++++ |
| I181L + V254T | ++++ |
| I181L + V255M | +++++ |
| V254T + V255M | ++++ |
| QES.i02 | +++++++++ |
| QES.i01 | +++++++++ |
| QES.i01 + I181L | +++++++++ |
| QES.i01 + V254T | +++++++++ |
| QES.i01 + I181L + V255M (QES.i01.c01) | ++++++++++ |
| YU2 Variant | 1.5 nM PC16 |
| Wildtype | +++ |
| V181L | +++ |
| V254T | +++ |
| V255M | +++ |
| 25711 Variant | 1.0 nM PC16 |
| Wildtype | +++ |
| I181L | +++++ |
| V254T | +++ |
| V255M | +++ |
| V254T + V255M | +++ |
| I181L + V254T (QES.c02) | +++++ |
| I181L + V255M | ++++ |
| I181L + V254T + V255M | ++++ |
| DU422 Variant | 1.5 nM PG16 |
| Wildtype | +++ |
| V181L | ++++++ |
| V254T | +++ |
| V255M | +++++ |
| V254T + V255M | +++++ |
| V181L + V254T | ++++++ |
| V181L + V255M | ++++++ |
| V181L + V254T + V255M (QES.c03) | +++++++ |
| Q769.d22 Variant | 1.5 nM PG16 |
| Wildtype | +++ |
| V181L | ++ |
| V254T | +++ |
| V255M | +++++ |
| V254T + V255M | +++++ |
| QES.i03 | ++++++++ |
| QES.i03 + V255M (QES.i03.V255M) | +++++++++ |
| Q842.d12 Variant | 30 nM PG16 |
| Wildtype | +++ |
| I181L | +++++ |
| V254T | +++ |
| V255M | +++++ |
| V254T + V255M | ++++ |
| I181L + V255M | ++++++ |
| I181L + V254T + V255M | ++++++ |
| QES.i04 | ++++++ |
| QES.i04 + I181L (QES.i04.I181L) | +++++++++ |
| QES.i04 + I181L + V255M | +++++ |

¹Qualitatively assessed by flow cytometry (n ≥ 2).
²+ is below 0.75 wildtype signal, ++ is 0.75-0.9x, +++ is 0.9-1.1, ++++ is 1.1-1.25x, +++++is 1.25-1.5x, ++++++ is 1.5-1.75x, +++++++ is 1.75-2x, ++++++++ is 2-2.5x, +++++++++ is 2.5-3x, and ++++++++++ is >3x wildtype binding signal.

Mutation I181L is a subtle mutation just below the surface of the apical tip where PG16 binds (FIG. 11D), perhaps stabilizing local packing. V254T and V255M are centrally positioned in the linker connecting the inner and outer domains of gp120. V254T adds a hydrogen bond to the backbone carbonyl of L261 in strand δ9 of the outer domain, while V255M increases hydrophobic packing to aromatic residues in the inner domain (FIG. 11D). The cavity occupied by V255M collapses in the open conformation with insufficient space for large hydrophobics (FIG. 11D), and the deep mutational scan predicts this mutation decreases CD4 interactions. (V255F was similarly found to increase PG16 binding while decreasing CD4 interactions.) V255M is therefore unique amongst the QES mutations described here in that it also destabilizes the open state.

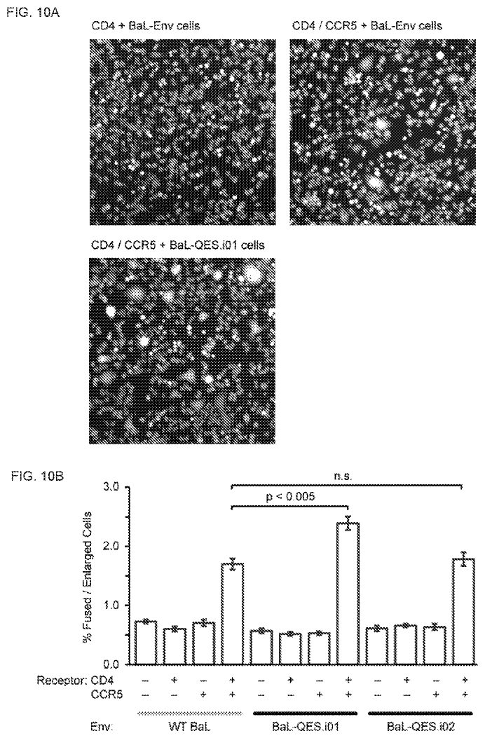

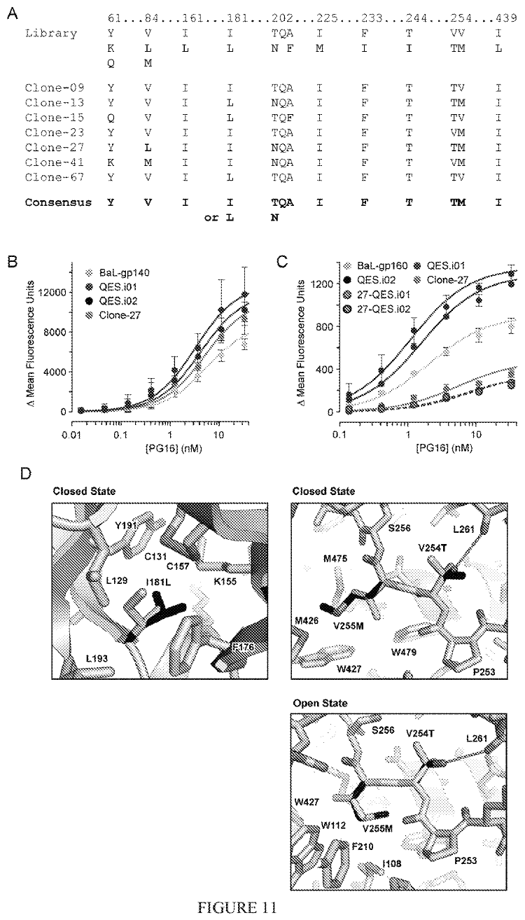

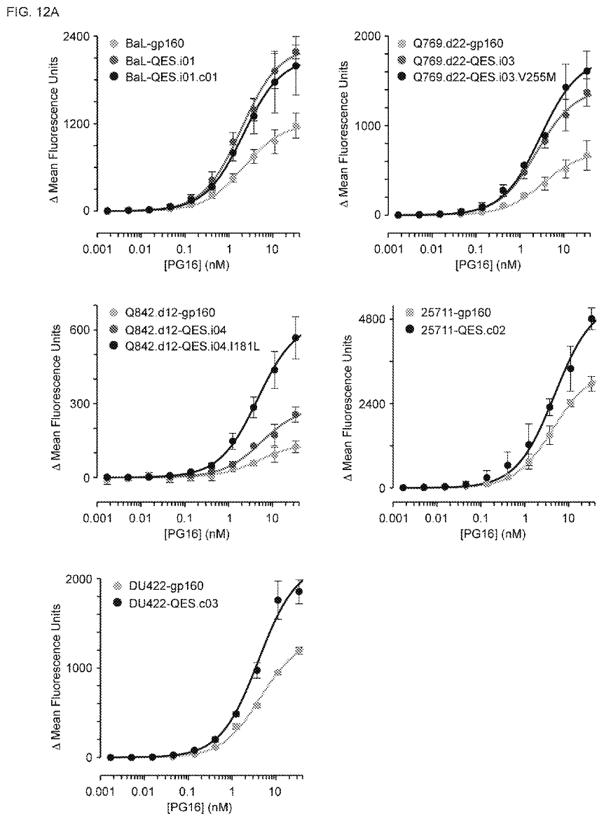

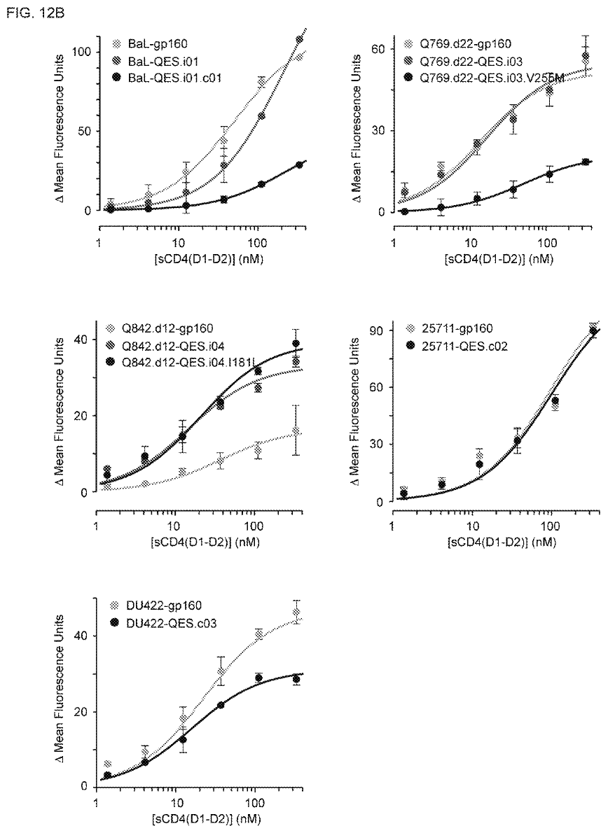

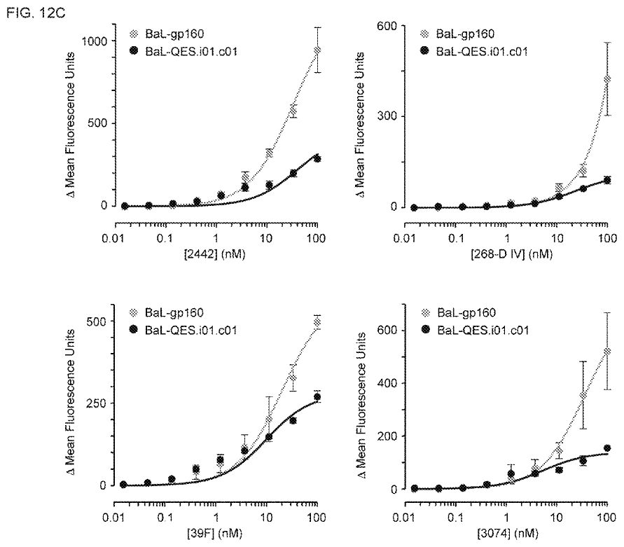

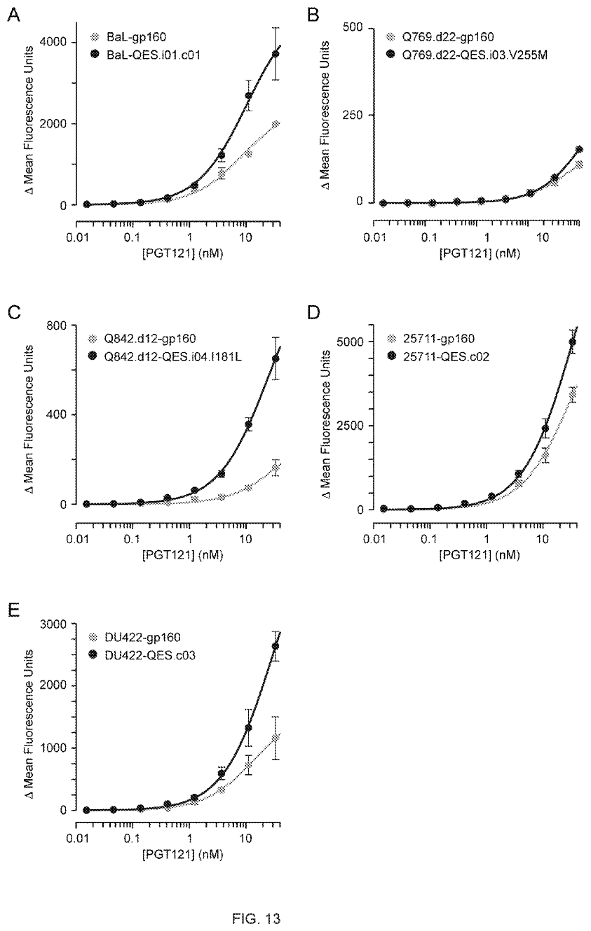

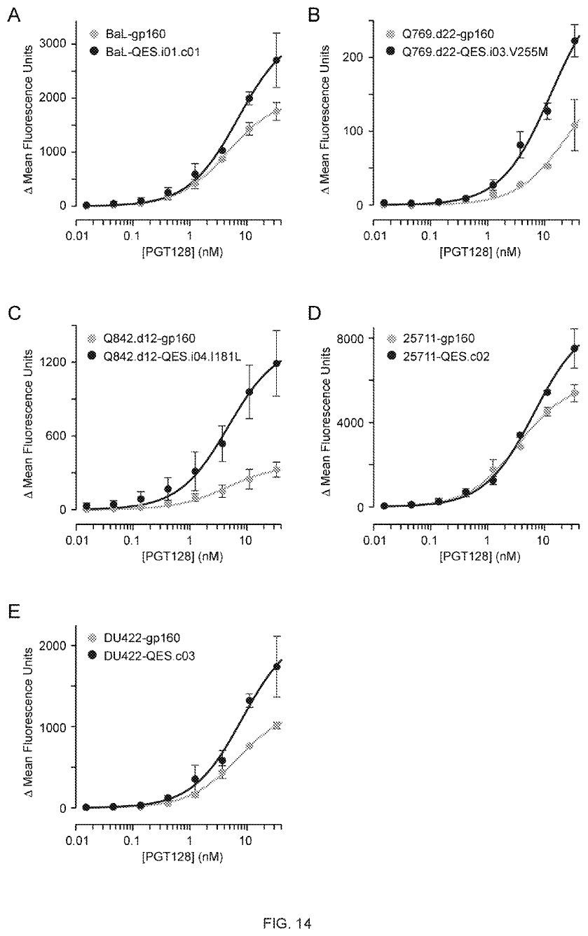

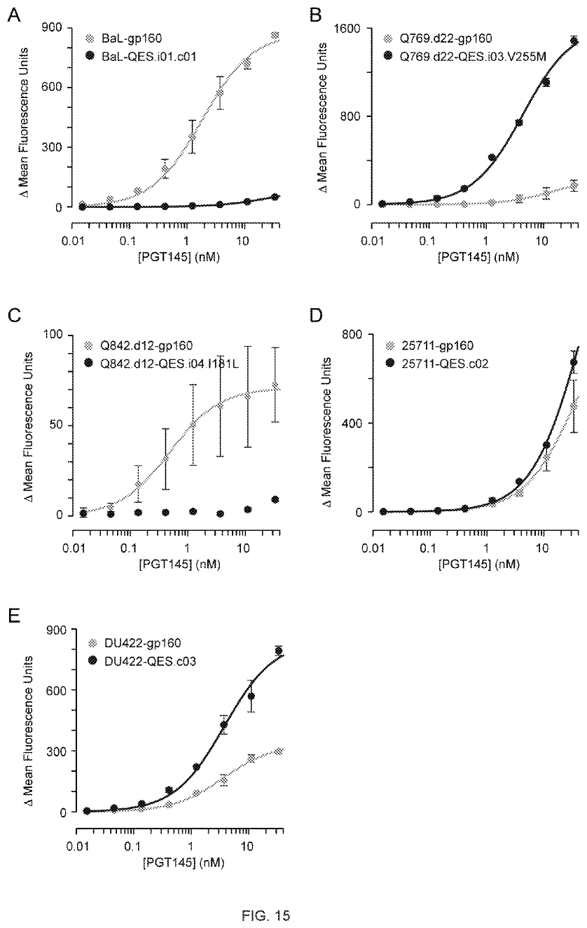

QES mutations within the core could be broadly applied to full-length Env from strains DU422, 25711, Q842.d12 and Q769.d22 (Table 4). Mutations were combined to create Env constructs BaL-QES.i01.c01 (“c for”, “core”, containing I181L and V255M), Q769-QES.i03.V255M, Q842-QES.i04.1181L, 25711-QES.c02 (I181L; V254T), and DU422-QES.c03 (V181L; V254T; V255M), which bound up to 5-fold more PG16 at saturation when expressed on the cell surface (FIG. 12A). Only variants harboring the V255M mutation had reduced CD4 binding (FIG. 12B), indicating that stabilization of the closed state alone is insufficient to prevent dynamic sampling or induction of the CD4-bound open state, which must instead be explicitly destabilized. In agreement, BaL-QES.i01.c01 Env now has reduced exposure of V3 region epitopes (FIG. 12C). Finally, the QES variants have increased presentation of the tertiary epitopes recognized by bNAbs PGT121 and PGT128 (FIGS. 13 and 14), which contact the N332 glycan supersite on the outer surface of gp120⁶⁴⁻⁶⁷. Hence using PG16 binding as a readout for Env conformational engineering simultaneously increases presentation of other complex structural epitopes. PGT145 binding increased or decreased in different QES constructs (FIG. 15), reflecting antagonism between stabilization of the PGT145-recognized closed conformation versus amino acid changes within the apical cavity that can disrupt direct antibody contacts.

The Env-QES variants mediated fusion to receptor-decorated membranes at levels similar to or greater than wildtype controls, unless the variant included the V255M substitution to destabilize the CD4-bound conformation, in which case membrane fusion was decreased (FIG. 16).

### Example 11. QES Mutations Stabilize Trimeric BG505 SOSIP

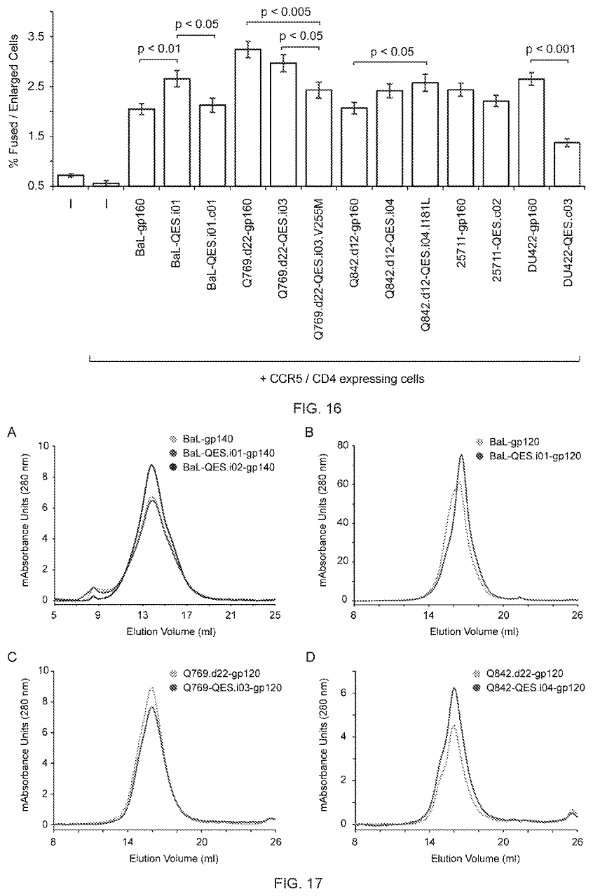

Size exclusion chromatography (SEC) was used to determine whether some of the QES mutations could shift purified soluble gp140 and gp120 towards higher molecular weight species, but observed no decrease in elution volume compared to the wildtype proteins (FIG. 17). This suggests the QES mutations alone may be insufficient for stabilizing trimers of extracellular Env fragments, and instead tested QES mutations in the BG505 SOSIP²⁰. Many of the QES mutations increased PG16 binding to cells expressing BG505 SOSIP anchored to the plasma membrane via a non-native TM helix (Table 5). Mutations were combined to generate BG505-QES.i03.c01 SOSIP (containing V181 L; A200E; F223Y; V255M; I595M).

TABLE 5
PG16 binding to QES mutants of BG505 SOSIP.664

| BG505 SOSIP.664 Variant (HXB2 numbering) | 1.5 nM PC16† |
|---|---|
| WT | +++ |
| Q114A | +++ |
| K117Y | ++++ |
| P124D | +++ |
| T163D | ++++ |
| R166L | +++ |
| V181L | ++++ |
| A200E | +++++ |
| F223Y | ++++ |
| V254T | +++ |
| V255M | ++++ |
| R557Q | +++ |
| L581D | +++ |
| I595M | ++++ |
| L663N | ++++ |
| K117Y + A200E | ++++ |
| T163D + A200E | ++++ |
| A200E + L581D | +++++ |
| A200E + R557Q | +++++ |
| A200E + I595M | ++++++ |
| K117Y + T163D | +++ |
| V181L + A200E | ++++++ |
| V181L + V255M | +++++ |
| V254T + V255M | ++++ |
| F223Y + L581D | +++ |
| K117Y + L581D | ++++ |
| A200E + F223Y + R557Q | +++++ |
| A200E + L581D + I595M | +++++ |
| A200E + F223Y + I595M (QES.i03) | ++++++ |
| A200E + L581D + I595M + L663N | ++++++ |
| K117Y + L581D + I595M | +++++ |
| K117Y + L581D + I595M + L663N | +++++ |
| A200E + F223Y + I595M + V181L | ++++++ |
| A200E + F223Y + I595M + V255M | +++++++ |
| A200E + F223Y + I595M + V181L + V255M (QES.i03c01) | +++++++ |

¹Qualitatively assessed by flow cytometry. Expi293F cells were transfected with mutant BG505 SOSIP.664 tethered to the membrane via the TM helix of HLA class 1α chain, and stained with 1.5 nM PG16. n = 2.
²++ is <0.9x wildtype binding signal, +++ is 0.9-1.1x, ++++ is 1.1-1.3x, +++++ is 1.3-1.5x, ++++++ is 1.5-1.7x, and +++++++ is 1.7-1.9x wildtype signal.

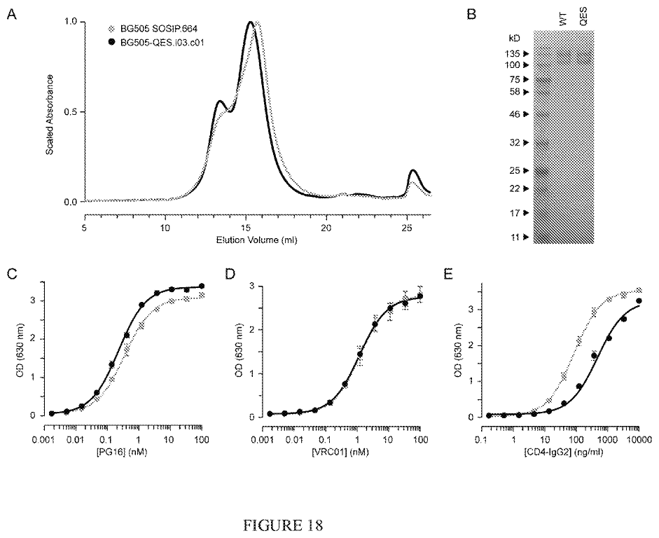

While SOSIP particles are generally purified using antibodies that recognize correctly folded trimers, 8his-tagged BG505-QES.i03.c1 SOSIP was instead purified by nickel affinity chromatography to inspect the full cohort of protein forms. Based on SEC, the protein fraction in the trimer peak was elevated in the QES variant, and monomer/dimer forms also showed slightly reduced elution volume suggestive of increased transient associations (FIG. 18A). PG16 binding to trimer fractions was slightly enhanced (FIG. 18C), and VRC01 bound wildtype and QES.i03.c01 BG505 SOSIP trimers equally (FIG. 18D). The apparent affinity for CD4-IgG2 was substantially reduced 5.5-fold (FIG. 18E), consistent with destabilization of the CD4-bound conformation. Proteolytic processing increased from ˜35% of wildtype to ˜50% of QES.i03.c01 (FIG. 18B). Hence while the deep mutational scan and subsequent engineering was focused on full-length Env, the mutations described herein are also applicable to a soluble extracellular construct.

### Example 12. Towards an Expanded Set of QES Mutations

Thus far, the application of deep mutational scanning to full-length Env from the clade B BaL strain has been described to qualitatively determine the impact of nearly all single amino acid substitutions on interactions with CD4 and the bNAbs VRC01⁴⁴ and PG16³⁹. By combining mutations (referred to as quatemary epitope stabilized or QES mutants) that enhance expression of a PG16-recognized closed trimer, Env was stabilized in a conformation/s that bound bNAbs PG16, PGT121⁶⁸, and PGT128^{29,68}. PG16-class antibodies bind at the Env apex between two protomers, explaining their strong preference for closed trimers⁴⁰. PGT121 and PGT128 recognize the N332 glycan supersite on the gp120 outer domain^{29,68}; antibodies targeting this site are frequently elicited bNAbs from multiple germline precursors, and therefore the N332 supersite may be an especially vulnerable epitope for the human antibody response to target^{65,68-71}. The majority of the QES mutations neutralized positive charge at the apical protomer interfaces, thereby reducing electrostatic repulsion between the apical tips that contributes to dynamic instability⁶¹. Furthermore, inclusion of a mutation (V255M) in the gp120 core to destabilize the open state reduced CD4 binding and exposure of V3 region epitopes. These mutations were effective in both full-length Env and soluble BG505 SOSIP.

It would be ideal if there existed a suite of mutations for applying broadly to any HIV-1 strain, which stabilize Env in a closed trimer for improved bNAb elicitation. A HIV-1 vaccine could then be rapidly modified and updated to contain stabilized Env sequences from local prevailing strains. However, the identified QES mutations in BaL Env were only partially transferable to other strains. This was especially true of QES mutations at subunit interfaces. Why is it that mutations to neutralize electropositive charge in the apical cavity of BaL or various clade A Env sequences stabilize a closed trimer, yet the equivalent mutations in Env from the clade C DU422 or 25711 strains do not? Are there different underlying features in primary structure that regulate the closed-to-open Env transition in different strains? To address this, sequence-activity landscapes of Env were determined from the DU422 strain for binding to CD4 and PG16.

### Example 13. A Deep Mutational Scan of DU422 gp140 for Binding to CD4 and PG16

A deep mutational scan, which involves using next generation sequencing to track the in vitro evolution or selection of a diverse library of sequence variants³⁴, requires a tight link between genotype and phenotype. For in vitro evolution of sequences under selection in human tissue culture, this can be accomplished by transfecting library DNA with a large excess of carrier DNA, such that a cell typically acquires no more than one coding sequence⁴⁷. A synthetic codon-optimized gene encoding gp160 from the DU422 strain was fused downstream of a CD5 leader sequence, and expressed from an episomal plasmid that can replicate extra-chromosomally. However, when transfected with a large excess of carrier DNA to limit the copy number of coding sequences acquired per cell, DU422 gp160 surface expression in Expi293F cells was not detected. Expression remained undetectable even when the coding sequence was downstream of an intron to promote transcript processing and nuclear export, or when gp160_{DU422} was cotransfected with carrier DNA designed to promote episomal plasmid replication. Since the cytoplasmic tail of gp160 features multiple motifs that regulate trafficking and endocytic turnover⁵⁰, only extracellular gp140_{DU422} (a.a. N31-N677) anchored to the membrane via a flexible gly/ser-rich linker and canonical transmembrane helix was expressed. Soluble gp140 has increased conformational heterogeneity compared to full-length Env^{21,72,73}, and therefore interesting mutations identified by the deep mutational scan are subsequently validated in the full protein. DU422 gp140 was expressed on the cell surface at high levels based on flow cytometric analysis of PG16 and soluble CD4 (sCD4: domains D1-D2) binding, even when diluted with excess carrier DNA during transfection.

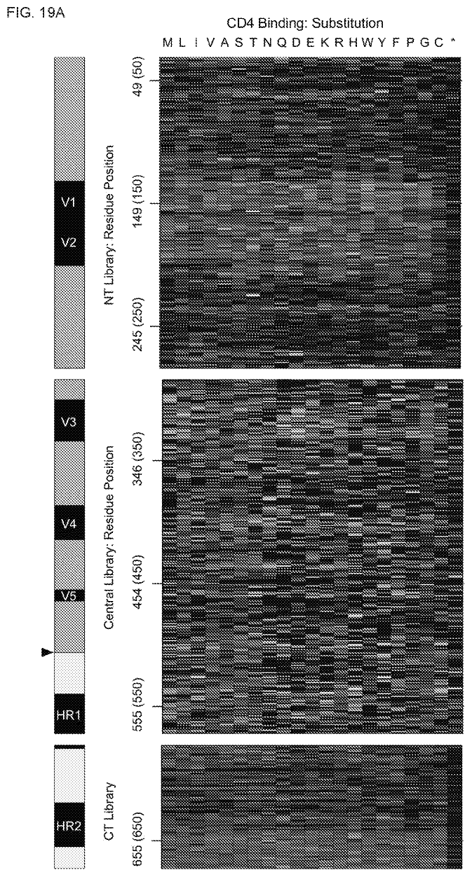

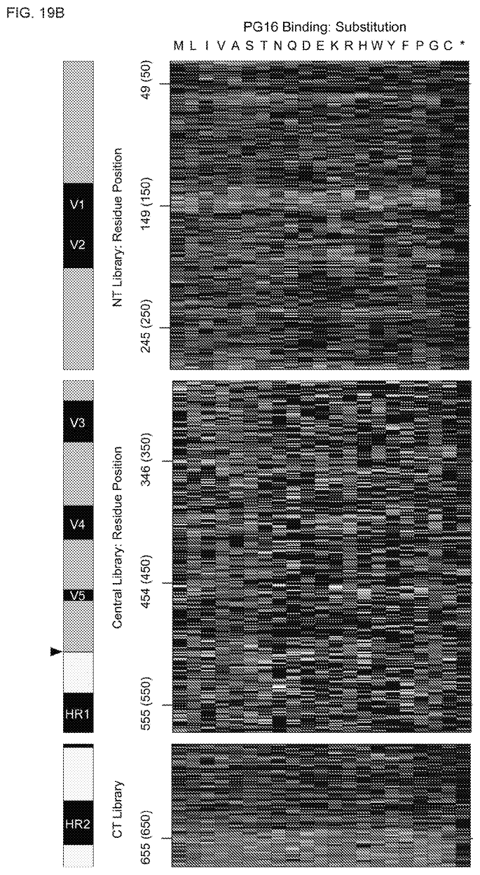

Three SSM libraries were prepared spanning gp140_{DU422} residues N31-N279 (NT library), N280-Q577 (central library), and T578-N677 (CT library). Together, the three libraries covered the full length of gp140_{DU422}, and encoded all 12,820 single amino acid substitutions. Expi293F cell cultures transiently expressing each library were fluorescently labeled with sCD4 or PG16, and cells expressing gp140 sequences with the highest ligand-binding signals were collected by FACS (Table 6). Cells were incubated with concentrations of sCD4 (10 nM) and PG16 (3 nM) below their apparent dissociation constants (18 and 5 nM, respectively), ensuring that mutants with higher or lower affinity could be distinguished by fluorescence intensity. RNA was extracted from the sorted cell populations, and gene-specific fragments were amplified from cDNA and deep sequenced. The frequencies of all mutations in the sorted populations were compared to the naïve libraries. Beneficial mutations increase in frequency and have positive log₂ enrichment ratios, whereas deleterious mutations are depleted and have negative log₂ enrichment ratios. The enrichment ratios are plotted in FIG. 19, and qualitatively define the sequence-activity landscapes for gp140_{DU422} interacting with CD4 (FIG. 19A) and PG16 (FIG. 19B).

TABLE 6
Sorting Conditions for gp140_{Du422} Deep Mutational Scanning.

| | | Collected Cells | |
|---|---|---|---|
| Ligand | Gating | Replicate 1 | Replicate 2 |
| sCD4(D1-D2) | Top 0.3% NT library | 112,000 | 115,000 |
| (10 nM) | Top 0.4% central library | 148,000 | 164,000 |
| | Top 0.4% CT library | 106,000 | 110,000 |
| PG16 | Top 0.6% NT library | 103,000 | 133,000 |
| (3 nM) | Top 0.6% central library | 142,000 | 140,000 |
| | Top 0.6% CT library | 81,000 | 61,000 |

### Example 14. Assessment of Data Quality

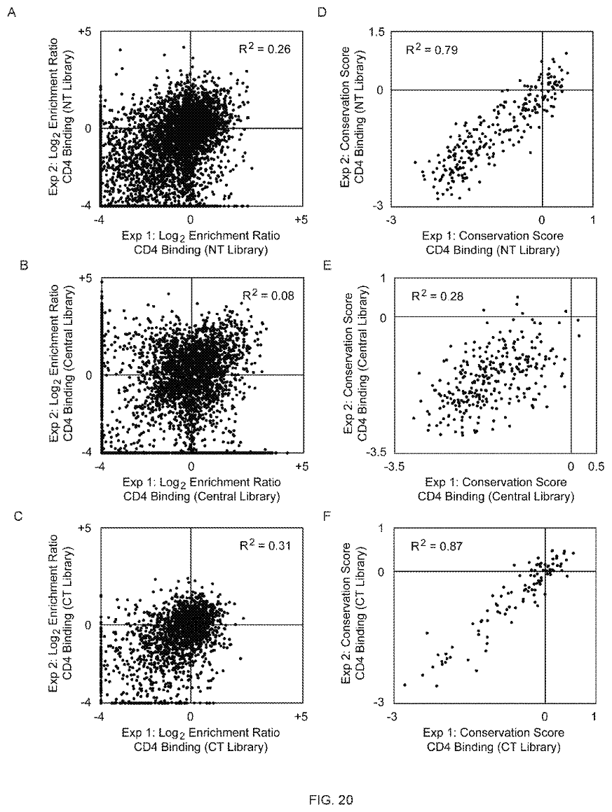

The sorting experiments were independently replicated, and the log₂ enrichment ratios for all mutations within the NT and CT libraries weakly agree; neutral mutations in the correlation plots are clustered near the origin, while deleterious mutations fall in the negative quadrant (FIGS. 20A, 20C, 20G, and 20I). Residue conservation scores, which are calculated by averaging the log₂ enrichment ratios for all twenty amino acids at each residue position, are tightly correlated between replicates (FIGS. 20D, 20F, 20J, and 20L) and define important functional sites with low mutational tolerance, either to maintain tight ligand interactions, fold correctly, or express on the cell surface. Data reproducibility is very similar to previous mutational scans of transmembrane proteins in human cells⁴⁷. However, the mutational scan across the central library is noisy with poor agreement between replicates, regardless of whether the library was sorted for sCD4 (FIG. 20B) or PG16 (FIG. 20H) binding signal. This was due to poor reverse transcription and PCR amplification across the central library during fragment preparation for deep sequencing, and could in future be resolved using alternative cDNA synthesis methods. Increased noise in the central library is further apparent as more extreme enrichment ratios in the sequence-activity landscapes (FIG. 19). Conservation scores across the central library therefore only approximate the mutational tolerance with some uncertainty (FIGS. 20E and 20K).

### Example 15. The Closed State of gp140_{DU422} Imposes Tight Conservation on the Apical Trimerization Domain

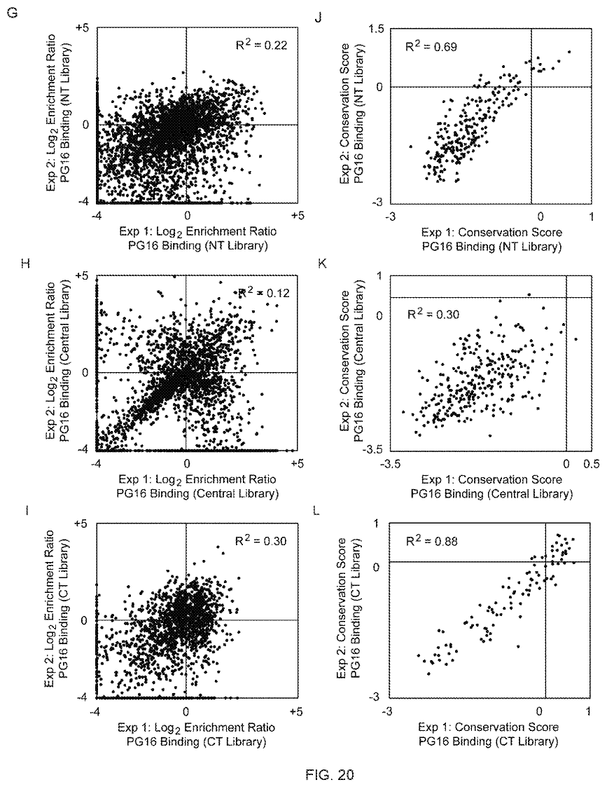

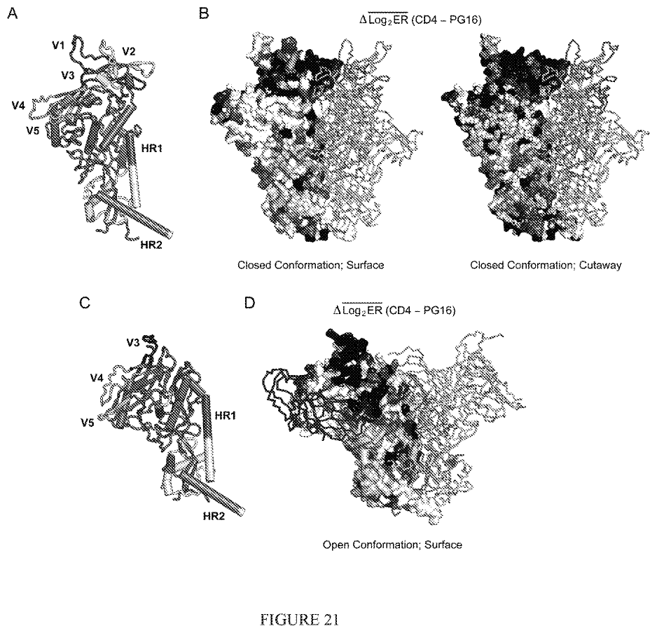

When the gp140_{DU422} sequence is under selection for sCD4 binding, diversity is tolerated in variable regions V1 to V5, and in residues downstream of heptad repeat HR2 that begin the membrane proximal extemal region or MPER (FIG. 19A). Strikingly, conservation extends into V1, V2, V3 and HR1 when gp140_{DU422} is selected for PG16 interactions (FIG. 19B). These regions make interprotomer contacts in trimeric Env⁶⁻¹¹. V1, V2 and V3 form the apical trimerization domain, while HR1 motifs from each protomer fold into a three-helix bundle that runs along the central C₃ axis. The stringent selection of residues at the trimer interface is further illustrated when conservation scores are mapped to atomic models of gp140_{DU422} in the closed and open states (FIG. 21). By taking the difference between the conservation scores for sCD4 and PG16 binding, residues preferentially conserved for acquiring the CD4-bound open state or PG16-bound closed state are highlighted; this analysis ‘masks’ or filters out residues under general conservation for folding and surface expression^{47,74}. Amino acids making direct atomic contacts with CD4^{13,56}, for example, are more conserved in the CD4 binding selections, as anticipated (FIG. 21D). PG16 primarily makes sequence-independent contacts to glycans on the upper apical surface that provide the antibody with broad strain reactivity⁶⁰, and yet gp140_{DU422} residues preferentially under tight conservation for PG16 binding span almost the entire trimer interface (FIG. 21B). High conservation in the PG16-CD4 difference map continues deep within the folded core of the apical trimerization domain, dramatically emphasizing that the V1, V2 and V3 regions are constrained in sequence space for adopting the trimeric PG16-recognized closed conformation.

High conservation within the trimerization domain of DU422 gp140 starkly contrasts with BaL gp160, where PG16 binding is not only more tolerant of mutations in V1, V2 and V3, but mutations in these regions can also enhance presentation of the PG16 quatemary epitope. This is likely due to both strain-specific sequence constraints, and differences between gp140 versus gp160. Extracellular gp140 has an unstable structure with substantial conformational diversity, and a sizeable protein fraction likely adopts non-native conformations^{18,21,72,73,75} The trimeric closed state of gp140, already destabilized, may therefore be more sensitive to mutational insults than full-length gp160. That said, a defining feature of the BaL gp160 sequence-activity landscape was that neutralization of electropositive charge at the apical trimer interface stabilized the PG16-bound closed conformation. This is not the case for DU422 gp140. For example, most substitutions to basic residues K117 and R166 at the apex are generally predicted to be neutral, and (excluding the central library where data quality is poor) overall very few substitutions are predicted from the mutational scan to increase PG16 binding. This agrees with prior targeted mutagenesis, where mutations that increased PG16 recognition of BaL gp160 did not have the same effect in DU422 gp160. These strain-specific features justify the decision to deep mutationally scan DU422 Env, as a more comprehensive set of quatemary epitope stabilizing (QES) mutations to favor closed trimers can now be identified from two disparate strains (BaL and DU422), increasing the applicability of QES mutations for engineering Env from any strain of interest for vaccine incorporation.

### Example 16. Mutations in DU422 gp160 at the Inner-Outer Domain Interface can Increase Expression of the PG16-Recognized Closed Trimer

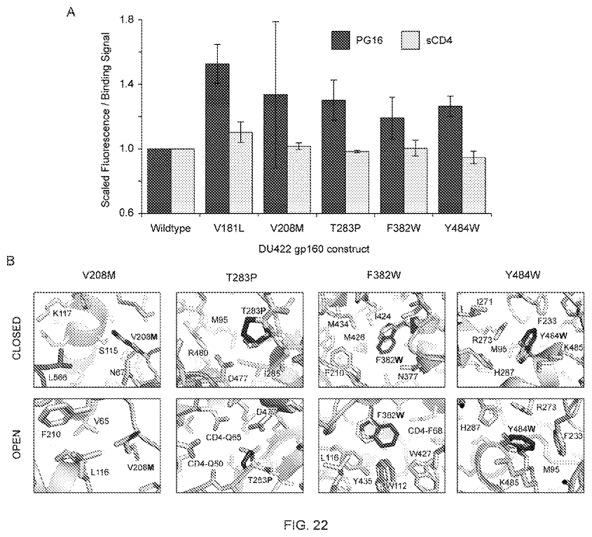

Based on predictions from the deep mutational scan, four new QES mutations were screened and validated in full-length gp160_{DU422} that show increased PG16 binding when expressed in Expi293F cells (FIG. 22A). Three of these mutations were identified from the mutational scan of the central SSM library, demonstrating that even when there is high uncertainty and noise, deep mutational scanning can be an effective screen for rare gain-of-function mutations. V208M is located at the interface between the gp120 inner and outer domains, a region containing the QES mutations V254T and V255M. V208M is directed in towards the inner domain and increases hydrophobic packing (FIG. 22B). F382W and Y484W both increase hydrophobic packing at the inner-outer domain interface (FIG. 22B). T283P is also located between the inner and outer domains, and while the mechanism by which T283P enhances PG16 binding is unclear, it may be that the hydrophobic proline disfavors open or partially open conformations where it becomes more solvent-accessible (FIG. 22B). The inner-outer domain interface undergoes substantial structural rearrangement upon CD4 ligation (FIG. 22B), and its stabilization may be an under-explored avenue for Env conformational engineering. Env F382 and Y484 are also universally conserved in all HIV-1 clades, raising the possibility that mutations to these residues in particular may be broadly effective in different strains.

### REFERENCES

1. Merk, A. & Subramaniam, S. HIV-1 envelope glycoprotein structure. Curr. Opin. Struct. Biol. 23, 268-276 (2013).
2. Huang, C.-C. et al. Structures of the CCR5 N terminus and of a tyrosine-sulfated antibody with HIV-1 gp120 and CD4. Science 317, 1930-1934 (2007).
3. Wu, L. et al. CD4-induced interaction of primary HIV-1 gp120 glycoproteins with the chemokine receptor CCR-5. Nature 384, 179-183 (1996).
4. Liu, J., Bartesaghi, A., Borgnia. M. J., Sapiro, G. & Subramaniam, S. Molecular architecture of native HIV-1 gp120 trimers. Nature 4566, 109-113 (2008).
5. Burton, D. R. et al. A Blueprint for HIV Vaccine Discovery. Cell Host Microbe 12, 396-407 (2012).
6. Wu, S.-R. et al. Single-particle cryoelectron microscopy analysis reveals the HIV-1 spike as a tripod structure. Proc. Natl. Acad. Sci. U.S.A. 107, 18844-18849 (2010).
7. White, T. A. et al. Molecular architectures of trimeric SIV and HIV-1 envelope glycoproteins on intact viruses: strain-dependent variation in quatemary structure. PLoS Pathog. 6, e1001249 (2010).
8. Julien, J.-P. et al. Crystal structure of a soluble cleaved HIV-1 envelope trimer. Science 342, 1477-1483 (2013).
9. Lyumkis, D. et al. Cryo-EM structure of a fully glycosylated soluble cleaved HIV-1 envelope trimer. Science 342, 1484-1490 (2013).
10. Mao, Y. et al. Subunit organization of the membrane-bound HIV-1 envelope glycoprotein trimer. Nat. Struct. Mol. Biol. 19, 893-899 (2012).
11. Pancera, M. et al. Structure and immune recognition of trimeric pre-fusion HIV-1 Env. Nature 514, 455-461 (2014).
12. Dev, J. et al. Structural basis for membrane anchoring of HIV-1 envelope spike. Science 363, 172-175 (2016).
13. Ozorowski, G. et al. Open and closed structures reveal allostery and pliability in the HIV-1 envelope spike. Nature 547, 360-363 (2017).
14. Munro, J. B. et al. Conformational dynamics of single HIV-1 envelope trimers on the surface of native virions. Science 346, 759-763 (2014).
15. McCoy, L. E. & Burton, D. R. Identification and specificity of broadly neutralizing antibodies against HIV. Immunol. Rev. 275, 11-20 (2017).
16. Sanders, R. W. et al. HIV-1 VACCINES. HIV-1 neutralizing antibodies induced by native-like envelope trimers. Science 349, aac4223-aac4223 (2015).
17. de Taeye, S. W. et al. Immunogenicity of Stabilized HIV-1 Envelope Trimers with Reduced Exposure of Non-neutralizing Epitopes. Cell 163, 1702-1715 (2015).
18. Binley, J. M. et al. A recombinant human immunodeficiency virus type 1 envelope glycoprotein complex stabilized by an intermolecular disulfide bond between the gp120 and gp41 subunits is an antigenic mimic of the trimeric virion-associated structure. J. Virol. 74, 627-643 (2000).
19. Sanders, R. W. et al. Stabilization of the soluble, cleaved, trimeric form of the envelope glycoprotein complex of human immunodeficiency virus type 1. J. Virol. 76, 8875-8889 (2002).
20. Sanders, R. W. et al. A next-generation cleaved, soluble HIV-1 Env trimer, BG505 SOSIP.664 gp140, expresses multiple epitopes for broadly neutralizing but not non-neutralizing antibodies. PLoS Pathog. 9, e1003618 (2013).
21. Sanders, R. W. & Moore, J. P. Native-like Env trimers as a platform for HIV-1 vaccine design. Immunol. Rev. 275, 161-182 (2017).
22. Kulp, D. W. et al. Structure-based design of native-like HIV-1 envelope trimers to silence non-neutralizing epitopes and eliminate CD4 binding. Nat Commun 8, 1655 (2017).
23. Kwon, Y. D. et al. Crystal structure, conformational fixation and entry-related interactions of mature ligand-free HIV-1 Env. Nat. Struct. Mol. Biol. 22, 522-531 (2015).
24. Steichen, J. M. et al. HIV Vaccine Design to Target Germline Precursors of Glycan-Dependent Broadly Neutralizing Antibodies. Immunity 45, 483-496 (2016).
25. Sarkar, A. et al. Structure of a cleavage-independent HIV Env recapitulates the glycoprotein architecture of the native cleaved trimer. Nat Commun 9, 1956 (2018).
26. Georgiev, I. S. et al. Single-Chain Soluble BG505.SOSIP gp140 Trimers as Structural and Antigenic Mimics of Mature Closed HIV-1 Env. J. Virol. 89, 5318-5329 (2015).
27. Sharma, S. K. et al. Cleavage-independent HIV-1 Env trimers engineered as soluble native spike mimetics for vaccine design. Cell Rep 11, 539-550 (2015).
28. Escolano, A. et al. Sequential Immunization Elicits Broadly Neutralizing Anti-HIV-1 Antibodies in Ig Knockin Mice. Cell 166, 1445-1458.e12 (2016).
29. Pejchal, R. et al. A potent and broad neutralizing antibody recognizes and penetrates the HIV glycan shield. Science 334, 1097-1103 (2011).
30. Jardine, J. et al. Rational HIV immunogen design to target specific germline B cell receptors. Science 340, 711-716 (2013).
31. McLellan, J. S. et al. Structure of HIV-1 gp120 V1/V2 domain with broadly neutralizing antibody PG9. Nature 480, 336-343 (2011).
32. Ofek, G. et al. Elicitation of structure-specific antibodies by epitope scaffolds. Proc. Natl. Acad. Sci. U.S.A. 107, 17880-17887 (2010).
33. Correia, B. E. et al. Computational design of epitope-scaffolds allows induction of antibodies specific for a poorly immunogenic HIV vaccine epitope. Structure 18, 1116-1126 (2010).
34. Fowler, D. M. & Fields, S. Deep mutational scanning: a new style of protein science. Nat. Methods 11, 801-807 (2014).
35. Jardine, J. G. et al. HIV-1 broadly neutralizing antibody precursor B cells revealed by germline-targeting immunogen. Science 351, 1458-1463 (2016).
36. Haddox, H. K., Dingens. A. S. & Bloom, J. D. Experimental Estimation of the Effects of All Amino-Acid Mutations to HIV's Envelope Protein on Viral Replication in Cell Culture. PLoS Pathog. 12, e1006114 (2016).
37. Dingens, A. S., Haddox, H. K., Overbaugh, J. & Bloom, J. D. Comprehensive Mapping of HIV-1 Escape from a Broadly Neutralizing Antibody. Cell Host Microbe 21, 777-787.e4 (2017).
38. Kovacs, J. M. et al. Stable, uncleaved HIV-1 envelope glycoprotein gp140 forms a tightly folded trimer with a native-like structure. Proc. Natl. Acad. Sci. U.S.A. 111, 18542-18547 (2014).
39. Walker, L. M. et al. Broad and potent neutralizing antibodies from an African donor reveal a new HIV-1 vaccine target. Science 326, 285-289 (2009).
40. Julien, J.-P. et al. Asymmetric recognition of the HIV-1 trimer by broadly neutralizing antibody PG9. Proc. Natl. Aced. Sci. U.S.A. 110, 4351-4356 (2013).
41. Tran. E. E. H. et al. Structural mechanism of trimeric HIV-1 envelope glycoprotein activation. PLoS Pathog. 85, e1002797 (2012).
42. Li, Y. et al. Mechanism of neutralization by the broadly neutralizing HIV-1 monoclonal antibody VRC01. J. Virol. 85, 8954-8967 (2011).
43. Moore, J. P., McKeating, J. A., Weiss, R. A. & Sattentau, Q. J. Dissociation of gp120 from HIV-1 virions induced by soluble CD4. Science 250, 1139-1142 (1990).
44. Wu, X. et al. Rational design of envelope identifies broadly neutralizing human monoclonal antibodies to HIV-1. Science 329, 856-861 (2010).
45. Seaman, M. S. et al. Tiered categorization of a diverse panel of HIV-1 Env pseudoviruses for assessment of neutralizing antibodies. J. Virol. 84, 1439-1452 (2010).
46. Haas, J., Park, E. C. & Seed, B. Codon usage limitation in the expression of HIV-1 envelope glycoprotein. Curr. Biol. 6, 315-324 (1996).
47. Heredia, J. D. et al. Mapping Interaction Sites on Human Chemokine Receptors by Deep Mutational Scanning. J. Immunol. ji1800343 (2018). doi:10.4049/jimmunol.1800343
48. Boge, M., Wyss, S., Bonifacino, J. S. & Thali, M. A membrane-proximal tyrosine-based signal mediates internalization of the HIV-1 envelope glycoprotein via interaction with the AP-2 clathrin adaptor. J. Biol. Chem. 273, 15773-15778 (1998).
49. LaBranche, C. C. et al. A single amino acid change in the cytoplasmic domain of the simian immunodeficiency virus transmembrane molecule increases envelope glycoprotein expression on infected cells. J. Virol. 69, 5217-5227 (1995).
50. Postler. T. S. & Desrosiers, R. C. The tale of the long tail: the cytoplasmic domain of HIV-1 gp41. J. Virol. 87, 2-15 (2013).
51. Yao, Q., Kuhlmann, F. M., Eller, R., Compans, R. W. & Chen, C. Production and characterization of simian-human immunodeficiency virus-like particles. AIDS Res. Hum. Retroviruses 16, 227-236 (2000).
52. Crooks, E. T. et al. Vaccine-Elicited Tier 2 HIV-1 Neutralizing Antibodies Bind to Quatemary Epitopes Involving Glycan-Deficient Patches Proximal to the CD4 Binding Site. PLoS Pathog. 11, e1004932 (2015).
53. Byland, R., Vance, P. J., Hoxie, J. A. & Marsh, M. A conserved dileucine motif mediates clathrin and AP-2-dependent endocytosis of the HIV-1 envelope protein. Mol. Biol. Cell 18, 414-425 (2007).
54. Kuhlmann, A.-S., Steckbeck, J. D., Sturgeon, T. J., Craigo, J. K. & Montelaro, R. C. Unique functional properties of conserved arginine residues in the lentivirus lytic peptide domains of the C-terminal tail of HIV-1 gp41. J. Biol. Chem. 289, 7630-7640 (2014).
55. Minezaki, Y., Homma, K. & Nishikawa, K. Intrinsically disordered regions of human plasma membrane proteins preferentially occur in the cytoplasmic segment. J. Mol. Biol. 368, 902-913 (2007).
56. Kwong, P. D. et al. Structure of an HIV gp120 envelope glycoprotein in complex with the CD4 receptor and a neutralizing human antibody. Nature 393, 648-659 (1998).
57. Myszka. D. G. et al. Energetics of the HIV gp120-CD4 binding reaction. Proc. Natl. Acad. Sci. U.S.A. 97, 9026-9031 (2000).
58. Zhou, T. et al. Structural basis for broad and potent neutralization of HIV-1 by antibody VRC01. Science 329, 811-817 (2010).
59. Peng, H.-P., Lee, K. H., Jian, J.-W. & Yang, A.-S. Origins of specificity and affinity in antibody-protein interactions. Proc. Natl. Acad. Sci. U.S.A. 111, E2656-65 (2014).
60. Pancera, M. et al. Structural basis for diverse N-glycan recognition by HIV-1-neutralizing V1-V2-directed antibody PG16. Nat. Struct. Mol. Biol. 20, 804-813 (2013).
61. Lee, J. H. et al. A Broadly Neutralizing Antibody Targets the Dynamic HIV Envelope Trimer Apex via a Long, Rigidified, and Anionic δ-Hairpin Structure. Immunity 46, 690-702 (2017).
62. Haim, H., Salas, I. & Sodroski, J. Proteolytic processing of the human immunodeficiency virus envelope glycoprotein precursor decreases conformational flexibility. J. Virol. 87, 1884-1889 (2013).
63. Grupping, K. et al. MiniCD4 protein resistance mutations affect binding to the HIV-1 gp120 CD4 binding site and decrease entry efficiency. Retrovirology 9, 36 (2012).
64. Lee, J. H., de Val, N., Lyumkis, D. & Ward, A. B. Model Building and Refinement of a Natively Glycosylated HIV-1 Env Protein by High-Resolution Cryoelectron Microscopy. Structure 23, 1943-1951 (2015).
65. Mouquet, H. et al. Complex-type N-glycan recognition by potent broadly neutralizing HIV antibodies. Proc. Natl. Acad. Sci. U.S.A. 109, E3268-77 (2012).
66. Games, F. et al. Structural evolution of glycan recognition by a family of potent HIV antibodies. Cell 159, 69-79 (2014).
67. Julien, J.-P. et al. Broadly neutralizing antibody PGT121 allosterically modulates CD4 binding via recognition of the HIV-1 gp120 V3 base and multiple surrounding glycans. PLoS Pathog. 9, e1003342 (2013).
68. Walker, L. M. et al. Broad neutralization coverage of HIV by multiple highly potent antibodies. Nature 477, 466-470 (2011).
69. MacLeod, D. T. et al. Early Antibody Lineage Diversification and Independent Limb Maturation Lead to Broad HIV-1 Neutralization Targeting the Env High-Mannose Patch. Immunity 44, 1215-1226 (2016).
70. Sok, D. et al. A Prominent Site of Antibody Vulnerability on HIV Envelope Incorporates a Motif Associated with CCR5 Binding and Its Camouflaging Glycans. Immunity 45, 31-45 (2016).
71. Kong, L. et al. Supersite of immune vulnerability on the glycosylated face of HIV-1 envelope glycoprotein gp120. Nat. Struct. Mol. Biol. 20, 796-803 (2013).
72. Jeffs, S. A. et al. Expression and characterisation of recombinant oligomeric envelope glycoproteins derived from primary isolates of HIV-1. Vaccine 22, 1032-1046 (2004).
73. Go, E. P. et al. Native Conformation and Canonical Disulfide Bond Formation Are Interlinked Properties of HIV-1 Env Glycoproteins. J. Virol. 90, 2884-2894 (2015).
74. Kowalsky, C. A. et al. Rapid Fine Conformational Epitope Mapping Using Comprehensive Mutagenesis and Deep Sequencing. J. Biol. Chem. 290, 26457-26470 (2015).
75. Ringe, R. P. et al. Cleavage strongly influences whether soluble HIV-1 envelope glycoprotein trimers adopt a native-like conformation. Proc. Natl. Acad. Sci. U.S.A. 110, 18256-18261 (2013).
76. Procko, E. et al. Computational design of a protein-based enzyme inhibitor. J. Mol. Biol. 425, 3563-3575 (2013).
77. Fowler, D. M., Araya, C. L., Gerard, W. & Fields, S. Enrich: software for analysis of protein function by enrichment and depletion of variants. Bioinformatics 27, 3430-3431 (2011).
78. Edgar, R., Domrachev. M. & Lash, A. E. Gene Expression Omnibus: NCBI gene expression and hybridization array data repository. Nucleic Acids Res. 30, 207-210 (2002).
79. Kwong, P. D. et al. HIV-1 evades antibody-mediated neutralization through conformational masking of receptor-binding sites. Nature 420, 678-682 (2002).
80. Pantophlet, R., Wilson, I. A. & Burton, D. R. Improved design of an antigen with enhanced specificity for the broadly HIV-neutralizing antibody b12. Protein Eng. Des. Sel. 17, 749-758 (2004).
81. Gorny, M. K. et al. Production of site-selected neutralizing human monoclonal antibodies against the third variable domain of the human immunodeficiency virus type 1 envelope glycoprotein. Proc. Natl. Acad. Sci. U.S.A. 88, 3238-3242 (1991).
82. Gorny, M. K. et al. Human monoclonal antibodies specific for conformation-sensitive epitopes of V3 neutralize human immunodeficiency virus type 1 primary isolates from various clades. J. Virol. 76, 9035-9045 (2002).
83. Gorny, M. K. et al. Cross-clade neutralizing activity of human anti-V3 monoclonal antibodies derived from the cells of individuals infected with non-B clades of human immunodeficiency virus type 1. J. Virol. 80, 6865-6872 (2006).
84. Hioe, C. E. et al. Anti-V3 monoclonal antibodies display broad neutralizing activities against multiple HIV-1 subtypes. PLoS ONE 5, e10254 (2010).
85. Stewart-Jones, G. B. E. et al. Trimeric HIV-1-Env Structures Define Glycan Shields from Clades A, B, and G. Cell 165, 813-826 (2016).
86. Cooper, S. et al. Predicting protein structures with a multiplayer online game. Nature 466, 756-760 (2010).
87. Leaver-Fay, A. et al. ROSETTA3: an object-oriented software suite for the simulation and design of macromolecules. Meth. Enzymol. 487, 545-574 (2011).
88. Nguyen, T. A. et al. Functional Anatomy of the Human Microprocessor. Cell 161, 1374-1387 (2015).

1. An HIV-1 Env protein or fragment thereof comprising one or more of the amino acid mutations listed below, wherein the amino acids are numbered by HXB2 numbering:
TABLE 1
T49D
Q114A
K117V
K117Y
P124D
T163D
R166E
R166F
R166L
I181L
V181L
V200E
V200T
A200E
V208M
F223Y
V254T
V255M
T283P
R315Q
R315A
F382W
K315A
R432T
Y484W
G514P
G516Q
R557Q
L581D
I595M
L663N

2. An HIV-1 Env protein or fragment thereof comprising one or more of the sets of amino acid mutations listed below, wherein the amino acids are numbered by HXB2 numbering:
TABLE 2
V200E + F223Y
R432T + R557Q
R315A + L663N
Q114A + V200T
K117V + T163D
K117V + R315A
Q114A + L663N
V200T + I595M
T49D + R315A + I595M
R166L + F223Y + L663N
K117V + R166L + R315A
K117V + R166L + F223Y
T163D + V200T + L581D
R166L + R315A + G514P
R315A + L663N + T49D
R315A + L663N + R166L
R315A + L663N + F223Y
R315A + L663N + R432T
R315A + L663N + I595M
T49D + P124D + I595M (QES.i01)
P124D + L663N
T49D + R315A + I595M + K117Y
T49D + R315A + I595M + R166L
T49D + R315A + I595M + L663N
T49D + K117V + R315A
K117V + R315A + L663N
K117V + R166L + F223Y + I595M
K117V + R166L + F223Y + L663N
P124D + R315A
P124D + R315A + L663N
T49D + R315A + I595M + L663N
T49D + P124D + R315A + I595M + L663N
T49D + P124D + R315A + I595M
T49D + P124D + I595M + L663N
T49D + P124D + R315A + G514P + I595M
T49D + P124D + L663N (QES.i02)
I181L + V254T (QES.c02)
I181L + V255M
V254T + V255M
T49D + P124D + I595M + I181L
T49D + P124D + I595M + V254T
T49D + P124D + I595M + I181L + V255M
(QES.i01.c01)
V181L + V254T
V181L + V255M
V181L + V254T + V255M (QES.c03)
P124D + I595M
P124D + F223Y + I595M
A200E + F223Y + I595M (QES.i03)
P124D + R557Q
R557Q + F223Y
P124D + A200E + F223Y + I595M
A200E + F223Y + R557Q + I595M
R166L + A200E
R166L + R557Q
R166L + R557Q + I595M
A200E + R557Q + I595M
P124D + A200E
P124D + R166L
P124D + R557Q + I595M
P124D + R166L + R557Q
R166L + A200E + R557Q
R166L + F223Y + R557Q
P124D + R166L + R557Q + I595M
P124D + F223Y + R557Q + I595M (QES.i04)
I181L + V254T + V255M
P124D + F223Y + R557Q + I595M + I181L
(QES.i04.I181L)
P124D + F223Y + R557Q + I595M +
I181L + V255M
K117Y + A200E
T163D + A200E
A200E + L581D
A200E + R557Q
A200E + I595M
V181L + A200E
K117Y + L581D
A200E + F223Y + R557Q
A200E + L581D + I595M
A200E + L581D + I595M + L663N
K117Y + L581D + I595M
K117Y + L581D + I595M + L663N
A200E + F223Y + I595M + V181L; and
A200E + F223Y + I595M + V181L + V255M
(QES.i03.c01)

3. A trimeric complex or portion thereof comprising HIV-1 Env proteins or fragments of claim 2 in a trimeric conformation.

4. An immunogen comprising one or more of the HIV-1 Env proteins or fragments thereof of claim 1.

5. A method of screening a compound for binding to one or more proteins or fragments thereof, wherein the one or more proteins or fragments thereof are selected from those in claim 1 comprising: providing the one or more proteins or fragments thereof; contacting the one or more proteins or fragments thereof with the compound; and determining the ability of the compound to bind to the one or more proteins or fragments thereof.

6. The method of claim 5, wherein the one or more proteins or fragments thereof comprise 2, 5, 10, 15, or more proteins or fragments thereof.

7. (canceled)

8. (canceled)

9. A library comprising two or more of the proteins or fragments thereof in claim 1.

10. A nucleic acid molecule encoding the HIV-1 Env protein or fragment thereof of claim 1.

11. A vector comprising the nucleic acid molecule of claim 10.

12. A host cell comprising the vector of claim 11.

13. A method of producing a protein comprising culturing the host cell of claim 12 in a culture medium to produce the protein.

14. The method of claim 13, wherein the host cell is a mammalian cell having the ability to glycosylate proteins.

15. A composition comprising one or more HIV-1 Env proteins or fragments thereof of claim 1, and a pharmaceutically acceptable carrier.

16. The composition of claim 15, further comprising an adjuvant.

17. A method for eliciting an immune response against an HIV-1 infected cell in a subject comprising administering to the subject an amount of the trimeric complex or portion thereof of claim 3, effective to elicit the immune response in the subject.

18. A method for preventing a subject from becoming infected with HIV-1 comprising administering to the subject a prophylactically effective amount of the trimeric complex or portion thereof of claim 3 such that the subject is prevented from becoming infected with HIV-1.

19. A method for reducing the likelihood of a subject becoming infected with HIV-1 comprising administering to the subject an amount of the trimeric complex or portion thereof of claim 3 effective to reduce the likelihood of the subject becoming infected with HIV-1.

20. The method of claim 19, wherein the subject has been exposed to HIV-1.

21. A method for delaying the onset of, or slowing the rate of progression of, an HIV-1-related disease or symptom in an HIV-1-infected subject comprising administering to the subject an amount of the trimeric complex or portion thereof of claim 3 effective to delay the onset of, or slow the rate of progression of the HIV-1-related disease or symptom in the subject.

22. The HIV-1 Env protein of claim 1, wherein the protein has at least one mutation shown in Table 1 and otherwise has about 95% or more sequence identity to an HIV-1 Env protein.

23. (canceled)

24. (canceled)

25. (canceled)

26. (canceled)


SEQUENCE LISTING (18 sequences)

SEQ ID NO: 1 - protein, 856 aa, Human immunodeficiency virus
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 2 - protein, 6 aa, Artificial Sequence
  Tag
DYKDDD

SEQ ID NO: 3 - protein, 8 aa, Artificial Sequence
  Linker
GSGSGGSG

SEQ ID NO: 4 - DNA, 20 bp, Artificial Sequence
  Synthetic oligonucleotide
cgttcagctgaacaatgatg

SEQ ID NO: 5 - DNA, 20 bp, Artificial Sequence
  Synthetic oligonucleotide
tgttggacaataccagacag

SEQ ID NO: 6 - DNA, 20 bp, Artificial Sequence
  Synthetic oligonucleotide
gtggtttgtccaaactcatc

SEQ ID NO: 7 - protein, 855 aa, Human immunodeficiency virus
MRVTEIRKSYQHWWRWGIMLLGILMICNAEEKLWVTVYYGVPVWKEATTTLFCASDRKAYDTEVHNVWATHACVPTDPNPQEVELKNVTENFNMWKNNMVEQMHEDIISLWDQSLKPCVKLTPLCVTLNCTDLRNATNGNDTNTTSSSRGMVGGGEMKNCSFNITTNIRGKVQKEYALFYKLDIAPIDNNSNNRYRLISCNTSVITQACPKVSFEPIPIHYCAPAGFAILKCKDKKFNGKGPCTNVSTVQCTHGIRPVVSTQLLLNGSLAEEEVVIRSANFADNAKVIIVQLNESVEINCTRPNNNTRKSIHIGPGRAFYTTGEIIGDIRQAHCNLSRAKWNDTLNKIVIKLREQFGNKTIVFKHSSGGDPEIVTHSFNCGGEFFYCNSTQLFNSTWNVTEESNNTVENNTITLPCRIKQIINMWQEVGRAMYAPPIRGQIRCSSNITGLLLTRDGGPEDNKTEVFRPGGGDMRDNWRSELYKYKVVKIEPLGVAPTKAKRRVVQREKRAVGIGAVFLGFLGAAGSTMGAAAMTLTVQARLLLSGIVQQQNNLLRAIEAQQHLLQLTVWGIKQLQARVLAVERYLRDQQLLGIWGCSGKLICTTAVPWNASWSNKSLNKIWDNMTWIEWDREINNYTSIIYSLIEESQNQQEKNEQELLELDKWASLWNWFDITKWLWYIKIFIMIVGGLIGLRIVFSVLSIVNRVRQGYSPLSFQTHLPSSRGPDRPGGIEEEGGERDRDRSGPLVNGFLALIWVDLRSLFLFSYHRLRDLLLIVMRIVELLGLAGGWEVLKYWWNLLQYWSQELKNSAVSLLNATAVAVAEGTDRVIEVLQRAVRAILHIPRRIRQGLERALL

SEQ ID NO: 8 - protein, 858 aa, Human immunodeficiency virus
MRVRGIPRNWPQWWIWGILGFWMIIICRVVGNLDLWVTVYYGVPVWKEAKTTLFCASDAKAYDKEVHNVWATHACVPTDPNPQEIVLENVTENFNMWKNDMVDQMHEDIISLWDQSLKPCVKLTPLCVTLNCKNVNISANANATATLNSSMNGEIKNCSFNTTTELRDKKQKVYALFYKPDVVPLNGGEHNETGEYILINCNSSTITQACPKVSFDPIPIHYCAPAGYAILKCNNKTFNGTGPCNNVSTVQCTHGIKPVVSTQLLLNGSLAEEEIIVRSENLTNNIKTIIVHLNKSVEIKCTRPNNNTRKSVRIGPGQTFYATGEIIGDIREAHCNISRETWNSTLIQVKEKLREHYNKTIKFEPSSGGDLEVTTHSFNCRGEFFYCDTTKLFNETKLFNESEYVDNKTIILPCRIKQIINMWQEVGRAMYAPPIEGNITCKSNITGLLLTWDGGENSTEGVFRPGGGNMKDNWRSELYKYKVVEIKPLGVAPTKSKRKVVGREKRAVGLGAVLLGFLGAAGSTMGAASITLTVQARQLLSGIVQQQSNLLRAIEAQQHLLQLTVWGIKQLQTRVLAIERYLKDQQLLGLWGCSGKLICATAVPWNSSWSNKSLGDIWDNMTWMQWDREISNYTNTIFRLLEDSQNQQEKNEKDLLALDSWKNLWNWFDITNWLWYIKIFIMIVGGLIGLRIIFGVLAIVKRVRQGYSPLSFQTLIPNPRGPDRLGRIEEEGGEQDKDRSIRLVSGFLALAWDDLRSLCLFSYHQLRDFILTAARAAELLGRSSLRGLQRGWEVLKYLGNLVQYWGLELKRSAINLFDTIAIAVAEGTDRIIEVIQRICRAIRYIPTRIRQGFEAALL

SEQ ID NO: 9 - protein, 855 aa, Human immunodeficiency virus
MRVKGTRKSYQQWWIWAVLGFWMLMICNVGGNLWVTVYYGVPVWKEAKTTLFCASDAKGYDKEVHNVWATHACVPTDPNPQEMPLENVTENFNMWENDMVNQMHEDVISLWDESLKPCVKLTPLCVTLNCTDVNKNVSSSDTDNYKETMKERKNCTFNMTTELRDKNQKKYALFYKLDIVPLDDNDNASYRLINCNTSTLTQACPKVSFDPIPIHYCAPAGYAILKCKNKTFNGIGPCNKVSTVQCTHGIKPVVSTQLLLNGSLAEEDIVIRSENITDNAKTIIVHLNESVEIVCIRPNNNTRKSIRIGPGQTFYATGDIVGDIRQAYCNISEGKWNKTLQRVSEKLAEHFPNSTINFNSSSGGDLEITTHSFNCGGEFFYCNTSGLFNGTYMNNDTKSNDTKSNSSSIITIPCRIKQIINMWQEVGRAVYAPPIAGNITCKSNITGILLTRDGGRGEEVKNDTETFRPGGGNMKDNWRSELYKYKVVEIKPLGVAPTAAKRRVVEREKRAVGLGAVLLGFLGAAGSTMGAASITLTVQARQLLSGIVQQQSNLLRAIEAQQHMLQLTVWGIKQLQARVLAIERYLKDQQLLGIWGCSGKLICTTAVPWNSSWSNKNQTEIWDKMTWMQWDREISNYTDTIYRLLEDSQNQQEKNEKDLLELDKWQNLWSWFNITNWLWYIRIFIMIVGGLIGLRIIFAVLSIVNRVRQGYSPLSFQTLAPNPRGLDRLGRIEEEGGKEDRNRSIRLVHGFLALAWDDLRSLCLFSYHRLRDLILLIARAVELLGQRGWEALKYLAGIVQYWGLELKKSAVSLFDTIAIAVAERTDRIIGLIQGICRAICNIPRRIRQGFEAALQ

SEQ ID NO: 10 - protein, 859 aa, Human immunodeficiency virus
MRAMGIQRNWQNLWRWGTMILGMILICCSAAGNLWVTVYYGVPVWRDAETTLFCASDAKAYDREAHNVWATHACVPTDPSPQEVPLGNVTEEFNMWKNNMVEQMHTDIISLWDQSLQPCVKLTPLCVTLNCSNSNNIPSVSNITDDMKEEIKNCSFNMTTELKDKKQNVYSLFYRLDVVPLETKTNQNSSHSRYRLINCNTSAITQACPKVSFEPIPIHYCAPAGFAILKCNDKGFNGTGLCKNVSTVQCTHGIKPVVSTQLLLNGSLAEGKVMVRSENITNNAKNIIIQFNNSVQINCTRPGNNTRKSIHLGPGKVFYATDIIGDIRKAHCNVNRQQWNKTLQDVATQLRTHFRNRTIIFNNSLGGDLEITTHSFNCRGEFFYCNTSGLFNGIWNGTQEPNRTESNDTITLQCRIKQIINMWQRVGQAIYAPPIQGEIRCESNITGLILTRDGGIINSTEETFRPGGGDMRDNWRSELYKYKVVKIEPLGVAPTKAKRRVVEREKRAVGFGAFFLGFLGAAGSTMGAASITLTVQARQLLSGIVQQQNNLLRAIEAQQHLLKLTVWGIKQLQARVLAVERYLKDQQLLGIWGCSGKFICTTTVPWNSSWSNKSQSEIWDNMTWMQWDKEINNYTQIIYDLIEESQRQQEKNEQDLLALDKWANLWNWFDISNWLWYIKIFIMIVGGLIGLRIAFAVLSVINRVRQGYSPLSFQTHTPNPRDLDRPGRIEEEGGEQDRDRSIRLVSGFLALAWDDLRSLCLFSYHRLRDFILVAARTVELLGHISLKGLRRGWEGLKYLGNLLSYWGRELKISAINLLDTIAIVVAEWTDRIIEIGQRLCRAIINIPRRIRQGFERALL

SEQ ID NO: 11 - protein, 852 aa, Human immunodeficiency virus
MRAMGIQMNCQNLWRWGTMILGMIIFCSAVDNLWVTVYYGVPVWKEAETTLFCASDAKAYETEKHNVWATHACVPTDPNPQEIHLENVTEEFNMWKNNMVEQMHTDIISLWDQSLKPCVKLTPLCVTLDCNNVTNNGTSDMREEIKNCSFNMTTELRDKRQKVYSLFYKLDIVQINEDQGNSSNNKYRLITCNTSAITQACPKVTFEPIPIHYCAPAGFAILKCKDEEFNGIGPCKNVSTVQCTHGIKPVVSTQLLLNGSLAEKEVKIRCENITNNAKTIIVQLVNPVKINCTRPNNNTRKSIHIGPGQAFYATGDIIGDIRQAHCNVNRTEWNNTLHQVVEQLRKHFNKTINFANSTGGDLEITTHSFNCGGEFFYCNTTNLFNSTWNHTASMNSTESNDTIILPCRIKQIINMWQRVGQAMYAPPIRGVIRCESNITGLILTRDGGNTNSTRETFRPGGGDMRDNWRSELYKYKVVKIEPLGVAPTKAKRRVVEREKRAVGIGAVFIGFLGAAGSTMGAASITLTVQARQLLSGIVQQQSNLLRAIEAQQHLLKLTVWGIKQLQARVLAVERYLKDQQLLGIWGCSGKLICTTSVPWNSSWSNKSQNEIWDNMTWLQWDKEISNYTQIIYDLLEESQNQQEKNEQDLLALDKWANLWNWFDISNWLWYIKIFIMIVGGLIGLRIVFAVLSVINRVRQGYSPLSFQTHTPNPRGLDRPERIEEEGGEQDKNRSIRLVSGFLALAWDDLRSLCLFSYHRLRDFILIVARTVELLGHSSLKGLRLGWEGLKYLGNLLSYWGRELRISATNLLDTIAIVIAGWTDRVIEIGQRLCRAFLNIPRRIRQGFERALL

SEQ ID NO: 12 - protein, 23 aa, Artificial Sequence
  Leader peptide
MPMGSLQPLATLYLLGMLVASVL

SEQ ID NO: 13 - protein, 850 aa, Artificial Sequence
  CD5-BaL gp160
MPMGSLQPLATLYLLGMLVASVLAEEKLWVTVYYGVPVWKEATTTLFCASDRKAYDTEVHNVWATHACVPTDPNPQEVELKNVTENFNMWKNNMVEQMHEDIISLWDQSLKPCVKLTPLCVTLNCTDLRNATNGNDTNTTSSSRGMVGGGEMKNCSFNITTNIRGKVQKEYALFYKLDIAPIDNNSNNRYRLISCNTSVITQACPKVSFEPIPIHYCAPAGFAILKCKDKKFNGKGPCTNVSTVQCTHGIRPVVSTQLLLNGSLAEEEVVIRSANFADNAKVIIVQLNESVEINCTRPNNNTRKSIHIGPGRAFYTTGEIIGDIRQAHCNLSRAKWNDTLNKIVIKLREQFGNKTIVFKHSSGGDPEIVTHSFNCGGEFFYCNSTQLFNSTWNVTEESNNTVENNTITLPCRIKQIINMWQEVGRAMYAPPIRGQIRCSSNITGLLLTRDGGPEDNKTEVFRPGGGDMRDNWRSELYKYKVVKIEPLGVAPTKAKRRVVQREKRAVGIGAVFLGFLGAAGSTMGAAAMTLTVQARLLLSGIVQQQNNLLRAIEAQQHLLQLTVWGIKQLQARVLAVERYLRDQQLLGIWGCSGKLICTTAVPWNASWSNKSLNKIWDNMTWIEWDREINNYTSIIYSLIEESQNQQEKNEQELLELDKWASLWNWFDITKWLWYIKIFIMIVGGLIGLRIVFSVLSIVNRVRQGYSPLSFQTHLPSSRGPDRPGGIEEEGGERDRDRSGPLVNGFLALIWVDLRSLFLFSYHRLRDLLLIVMRIVELLGLAGGWEVLKYWWNLLQYWSQELKNSAVSLLNATAVAVAEGTDRVIEVLQRAVRAILHIPRRIRQGLERALL

SEQ ID NO: 14 - protein, 851 aa, Artificial Sequence
  CD5-DU422 gp160
MPMGSLQPLATLYLLGMLVASVLANLDLWVTVYYGVPVWKEAKTTLFCASDAKAYDKEVHNVWATHACVPTDPNPQEIVLENVTENFNMWKNDMVDQMHEDIISLWDQSLKPCVKLTPLCVTLNCKNVNISANANATATLNSSMNGEIKNCSFNTTTELRDKKQKVYALFYKPDVVPLNGGEHNETGEYILINCNSSTITQACPKVSFDPIPIHYCAPAGYAILKCNNKTFNGTGPCNNVSTVQCTHGIKPVVSTQLLLNGSLAEEEIIVRSENLTNNIKTIIVHLNKSVEIKCTRPNNNTRKSVRIGPGQTFYATGEIIGDIREAHCNISRETWNSTLIQVKEKLREHYNKTIKFEPSSGGDLEVTTHSFNCRGEFFYCDTTKLFNETKLFNESEYVDNKTIILPCRIKQIINMWQEVGRAMYAPPIEGNITCKSNITGLLLTWDGGENSTEGVFRPGGGNMKDNWRSELYKYKVVEIKPLGVAPTKSKRKVVGREKRAVGLGAVLLGFLGAAGSTMGAASITLTVQARQLLSGIVQQQSNLLRAIEAQQHLLQLTVWGIKQLQTRVLAIERYLKDQQLLGLWGCSGKLICATAVPWNSSWSNKSLGDIWDNMTWMQWDREISNYTNTIFRLLEDSQNQQEKNEKDLLALDSWKNLWNWFDITNWLWYIKIFIMIVGGLIGLRIIFGVLAIVKRVRQGYSPLSFQTLIPNPRGPDRLGRIEEEGGEQDKDRSIRLVSGFLALAWDDLRSLCLFSYHQLRDFILTAARAAELLGRSSLRGLQRGWEVLKYLGNLVQYWGLELKRSAINLFDTIAIAVAEGTDRIIEVIQRICRAIRYIPTRIRQGFEAALL

SEQ ID NO: 15 - protein, 848 aa, Artificial Sequence
  CD5-25711 gp160
MPMGSLQPLATLYLLGMLVASVLANLWVTVYYGVPVWKEAKTTLFCASDAKGYDKEVHNVWATHACVPTDPNPQEMPLENVTENFNMWENDMVNQMHEDVISLWDESLKPCVKLTPLCVTLNCTDVNKNVSSSDTDNYKETMKERKNCTFNMTTELRDKNQKKYALFYKLDIVPLDDNDNASYRLINCNTSTLTQACPKVSFDPIPIHYCAPAGYAILKCKNKTFNGIGPCNKVSTVQCTHGIKPVVSTQLLLNGSLAEEDIVIRSENITDNAKTIIVHLNESVEIVCIRPNNNTRKSIRIGPGQTFYATGDIVGDIRQAYCNISEGKWNKTLQRVSEKLAEHFPNSTINFNSSSGGDLEITTHSFNCGGEFFYCNTSGLFNGTYMNNDTKSNDTKSNSSSIITIPCRIKQIINMWQEVGRAVYAPPIAGNITCKSNITGILLTRDGGRGEEVKNDTETFRPGGGNMKDNWRSELYKYKVVEIKPLGVAPTAAKRRVVEREKRAVGLGAVLLGFLGAAGSTMGAASITLTVQARQLLSGIVQQQSNLLRAIEAQQHMLQLTVWGIKQLQARVLAIERYLKDQQLLGIWGCSGKLICTTAVPWNSSWSNKNQTEIWDKMTWMQWDREISNYTDTIYRLLEDSQNQQEKNEKDLLELDKWQNLWSWFNITNWLWYIRIFIMIVGGLIGLRIIFAVLSIVNRVRQGYSPLSFQTLAPNPRGLDRLGRIEEEGGKEDRNRSIRLVHGFLALAWDDLRSLCLFSYHRLRDLILLIARAVELLGQRGWEALKYLAGIVQYWGLELKKSAVSLFDTIAIAVAERTDRIIGLIQGICRAICNIPRRIRQGFEAALQ

SEQ ID NO: 16 - protein, 853 aa, Artificial Sequence
  CD5-Q769.d22 gp160
MPMGSLQPLATLYLLGMLVASVLAAGNLWVTVYYGVPVWRDAETTLFCASDAKAYDREAHNVWATHACVPTDPSPQEVPLGNVTEEFNMWKNNMVEQMHTDIISLWDQSLQPCVKLTPLCVTLNCSNSNNIPSVSNITDDMKEEIKNCSFNMTTELKDKKQNVYSLFYRLDVVPLETKTNQNSSHSRYRLINCNTSAITQACPKVSFEPIPIHYCAPAGFAILKCNDKGFNGTGLCKNVSTVQCTHGIKPVVSTQLLLNGSLAEGKVMVRSENITNNAKNIIIQFNNSVQINCTRPGNNTRKSIHLGPGKVFYATDIIGDIRKAHCNVNRQQWNKTLQDVATQLRTHFRNRTIIFNNSLGGDLEITTHSFNCRGEFFYCNTSGLFNGIWNGTQEPNRTESNDTITLQCRIKQIINMWQRVGQAIYAPPIQGEIRCESNITGLILTRDGGIINSTEETFRPGGGDMRDNWRSELYKYKVVKIEPLGVAPTKAKRRVVEREKRAVGFGAFFLGFLGAAGSTMGAASITLTVQARQLLSGIVQQQNNLLRAIEAQQHLLKLTVWGIKQLQARVLAVERYLKDQQLLGIWGCSGKFICTTTVPWNSSWSNKSQSEIWDNMTWMQWDKEINNYTQIIYDLIEESQRQQEKNEQDLLALDKWANLWNWFDISNWLWYIKIFIMIVGGLIGLRIAFAVLSVINRVRQGYSPLSFQTHTPNPRDLDRPGRIEEEGGEQDRDRSIRLVSGFLALAWDDLRSLCLFSYHRLRDFILVAARTVELLGHISLKGLRRGWEGLKYLGNLLSYWGRELKISAINLLDTIAIVVAEWTDRIIEIGQRLCRAIINIPRRIRQGFERALL

SEQ ID NO: 17 - protein, 847 aa, Artificial Sequence
  CD5-Q842.d12 gp160
MPMGSLQPLATLYLLGMLVASVLAVDNLWVTVYYGVPVWKEAETTLFCASDAKAYETEKHNVWATHACVPTDPNPQEIHLENVTEEFNMWKNNMVEQMHTDIISLWDQSLKPCVKLTPLCVTLDCNNVTNNGTSDMREEIKNCSFNMTTELRDKRQKVYSLFYKLDIVQINEDQGNSSNNKYRLITCNTSAITQACPKVTFEPIPIHYCAPAGFAILKCKDEEFNGIGPCKNVSTVQCTHGIKPVVSTQLLLNGSLAEKEVKIRCENITNNAKTIIVQLVNPVKINCTRPNNNTRKSIHIGPGQAFYATGDIIGDIRQAHCNVNRTEWNNTLHQVVEQLRKHFNKTINFANSTGGDLEITTHSFNCGGEFFYCNTTNLFNSTWNHTASMNSTESNDTIILPCRIKQIINMWQRVGQAMYAPPIRGVIRCESNITGLILTRDGGNTNSTRETFRPGGGDMRDNWRSELYKYKVVKIEPLGVAPTKAKRRVVEREKRAVGIGAVFIGFLGAAGSTMGAASITLTVQARQLLSGIVQQQSNLLRAIEAQQHLLKLTVWGIKQLQARVLAVERYLKDQQLLGIWGCSGKLICTTSVPWNSSWSNKSQNEIWDNMTWLQWDKEISNYTQIIYDLLEESQNQQEKNEQDLLALDKWANLWNWFDISNWLWYIKIFIMIVGGLIGLRIVFAVLSVINRVRQGYSPLSFQTHTPNPRGLDRPERIEEEGGEQDKNRSIRLVSGFLALAWDDLRSLCLFSYHRLRDFILIVARTVELLGHSSLKGLRLGWEGLKYLGNLLSYWGRELRISATNLLDTIAIVIAGWTDRVIEIGQRLCRAFLNIPRRIRQGFERALL

SEQ ID NO: 18 - protein, 24 aa, Artificial Sequence
  Leader peptide
MPMGSLQPLATLYLLGMLVASVLA